(19) United States
(12) Patent Application Publication
(10) Pub. No.: US 2009/0255004 A1
(43) Pub. Date: Oct. 8, 2009

(54) G-Protein Coupled Receptor and Uses Therefor

(75) Inventors: Maria Blatcher, Moorestown, NJ (US); Brian Gaither Bates, Chelmsford, MA (US); Janet Elizabeth Paulsen, Londonderry, NH (US)

Correspondence Address: CHOATE, HALL & STEWART LLP/WYETH, PATENT GROUP, TWO INTERNATIONAL PLACE, BOSTON, MA, 02110, US

(73) Assignee: WYETH, Madison, NJ (US)

(21) Appl. No.: 12/397,761

(22) Filed: Mar. 4, 2009

Related U.S. Application Data
(63) Continuation of application No. 11/396,960, filed on Apr. 3, 2006, now abandoned.
Application No. 11/396,960 is a continuation of application No. 10/166,221, filed on Jun. 7, 2002, now abandoned.
(60) Provisional application No. 60/297,131, filed on Jun. 7, 2001.

(51) Int. Cl. A01K 67/027; C12N 15/12; C12N 15/85; C07K 14/47; C07K 16/18; G01N 33/566; C12Q 1/68; C12N 5/00

(52) U.S. Cl. 800/14; 536/23.5; 435/320.1; 530/350; 530/387.9; 436/501; 435/6; 435/375

(57) ABSTRACT

The present invention is based on the identification of a G-protein coupled receptor (GPCR) that is expressed predominantly in the brain and placenta and nucleic acid molecules that encoded the GPCR, which is referred to herein as the hCAR protein and hCAR gene respectively (for human Constitutively Active Receptor). Based on this identification, the present invention provides: (1) isolated hCAR protein; (2) isolated nucleic acid molecules that encode an hCAR protein; (3) antibodies that selectively bind to the hCAR protein; (4) methods of isolating allelic variants of the hCAR protein and gene; (5) methods of identifying cells and tissues that express the hCAR protein/gene; (6) methods of identifying agents and cellular compounds that bind to the hCAR protein; (7) methods of identifying agents that modulate the expression of the hCAR gene; and (8) methods of modulating the activity of the hCAR protein in a cell or organism.

### RELATED APPLICATIONS

This application claims priority from copending provisional application Ser. No. 60/297,131, filed on Jun. 7, 2001, the contents of which are hereby incorporated in their entirety by reference.

### FIELD OF THE INVENTION

The present invention relates generally to the fields of neuroscience, bioinformatics and molecular biology. More particularly, the invention relates to newly identified polynucleotides that encode a G-protein coupled receptor (GPCR) which has been designated human Constitutively Active Receptor (hCAR), the use of such polynucleotides and polypeptides, as well as the production of such polynucleotides and polypeptides. The invention also relates to identifying compounds which may be agonists, antagonists and/or inhibitors of GPCRs, and therefore potentially useful in therapy.

### BACKGROUND OF THE INVENTION

G-protein coupled receptors (GPCRs) are proteins that have seven transmembrane domains. Upon binding of a ligand to a GPCR, a signal is transduced within the cell, which results in a change in a biological or physiological property of the cell.

GPCRs, along with G-proteins and effectors (intracellular enzymes and channels which are modulated by G-proteins), are the components of a modular signaling system that connects the state of intracellular second messengers to extracellular inputs. These genes and gene-products are potential causative agents of disease.

Specific defects in the rhodopsin gene and the V2 vasopressin receptor gene have been shown to cause various forms of autosomal dominant and autosomal recessive retinitis pigmentosa, nephrogenic diabetes insipidus. These receptors are of critical importance to both the central nervous system and peripheral physiological processes. The GPCR protein superfamily now contains over 250 types of paralogues, receptors that represent variants generated by gene duplications (or other processes), as opposed to orthologues, the same receptor from different species. The superfamily can be broken down into five families: Family I, receptors typified by rhodopsin and the beta2-adrenergic receptor and currently represented by over 200 unique members; Family II, the recently characterized parathyroid hormone/calcitonin/secretin receptor family; Family. III, the metabotropic glutamate receptor family in mammals; Family IV, the cAMP receptor family, important in the chemotaxis and development of D. discoideum; and Family V, the fungal mating pheromone receptors such as STE2.

GPCRs include receptors for biogenic amines, for lipid mediators of inflammation, peptide hormones, and sensory signal mediators. The GPCR becomes activated when the receptor binds its extracellular ligand. Conformational changes in the GPCR, which result from the ligand-receptor interaction, affect the binding affinity of a G protein to the GPCR intracellular domains. This enables GTP to bind with enhanced affinity to the G protein.

Activation of the G protein by GTP leads to the interaction of the G protein α subunit with adenylate cyclase or other second messenger molecule generators. This interaction regulates the activity of adenylate cyclase and hence production of a second messenger molecule, cAMP. cAMP regulates phosphorylation and activation of other intracellular proteins. Alternatively, cellular levels of other second messenger molecules, such as cGMP or eicosinoids, may be upregulated or downregulated by the activity of GPCRs. The G protein a subunit is deactivated by hydrolysis of the GTP by GTPase, and the α, β, and γ subunits reassociate. The heterotrimeric G protein then dissociates from the adenylate cyclase or other second messenger molecule generator. Activity of GPCR may also be regulated by phosphorylation of the intra- and extracellular domains or loops.

Glutamate receptors form a group of GPCRs that are important in neurotransmission. Glutamate is the major neurotransmitter in the CNS and is believed to have important roles in neuronal plasticity, cognition, memory, learning and some neurological disorders such as epilepsy, stroke, and neurodegeneration (Watson, S. and Arkinstall, S. (1994) The G-Protein Linked Receptor Facts Book, Academic Press, San Diego Calif., pp. 130-132). These effects of glutamate are mediated by two distinct classes of receptors termed ionotropic and metabotropic. Ionotropic receptors contain an intrinsic cation channel and mediate fast excitatory actions of glutamate. Metabotropic receptors are modulatory, increasing the membrane excitability of neurons by inhibiting calcium dependent potassium conductances and both inhibiting and potentiating excitatory transmission of ionotropic receptors. Metabotropic receptors are classified into five subtypes based on agonist pharmacology and signal transduction pathways and are widely distributed in brain tissues.

The vasoactive intestinal polypeptide (VIP) family is a group of related polypeptides whose actions are also mediated by GPCRs. Key members of this family are VIP itself, secretin, and growth hormone releasing factor (GRF). VIP has a wide profile of physiological actions including relaxation of smooth muscles, stimulation or inhibition of secretion in various tissues, modulation of various immune cell activities. and various excitatory and inhibitory activities in the CNS. Secretin stimulates secretion of enzymes and ions in the pancreas and intestine and is also present in small amounts in the brain. GRF is an important neuroendocrine agent regulating synthesis and release of growth hormone from the anterior pituitary (Watson, S. and Arkinstall, S. supra, pp. 278-283).

Following ligand binding to the GPCR, a conformational change is transmitted to the G protein, which causes the α-subunit to exchange a bound GDP molecule for a GTP molecule and to dissociate from the βγ-subunits. The GTP-bound form of the α-subunit typically functions as an effector-modulating moiety, leading to the production of second messengers, such as cyclic AMP (e.g., by activation of adenylate cyclase), diacylglycerol or inositol phosphates. Greater than 20 different types of α-subunits are known in man, which associate with a smaller pool of β and γ subunits. Examples of mammalian G proteins include Gi, Go, Gq, Gs and Gt. G proteins are described extensively in Lodish, H. et al. Molecular Cell Biology, (Scientific American Books Inc., New York, N.Y., 1995), the contents of which is incorporated herein by reference.

GPCRs are a major target for drug action and development. In fact, receptors have led to more than half of the currently known drugs (Drews, Nature Biotechnology, 1996, 14: 1516) and GPCRs represent the most important target for therapeutic intervention with 30% of clinically prescribed drugs either antagonizing or agonizing a GPCR (Milligan, G. and Rees, S., (1999) TIPS, 20: 118-124) This demonstrates that these receptors have an established, proven history as therapeutic targets. The hCAR GPCR described in this invention clearly satisfies a need in the art for identification and characterization of further receptors that can play a role in diagnosing, preventing, ameliorating or correcting dysfunctions, disorders, or diseases.

In particular, the hCAR GPCR described in this invention satisfies a need in the art for identification and characterization of further receptors that can play an important role in diagnosing, preventing, ameliorating or correcting psychiatric and CNS dysfunctions, disorders, or diseases.

The present invention advances the state of the art by providing a GPCR which is expressed predominantly in the brain and placenta.

### SUMMARY OF THE INVENTION

The present invention is based on the identification of a G-protein coupled receptor (GPCR) that is expressed predominantly in the brain and the placenta and nucleic acid molecules that encoded the GPCR, referred to herein as the hCAR protein and hCAR cDNA respectively. The hCAR sequence in the genome is referred to as the hCAR gene. The present invention provides: isolated hCAR protein; nucleic acid molecules that encode an hCAR protein; antibodies that selectively bind to the hCAR protein; methods of isolating allelic variants of the hCAR protein and gene; methods of identifying cells and tissues that express the hCAR protein/gene; methods of identifying agents and cellular compounds that bind to the hCAR protein; methods of identifying agents that modulate the expression of the hCAR gene; methods of modulating the activity of the hCAR protein in a cell or organism; transgenic non-human animals expressing hCAR; knockout non-human animals with altered hCAR expression; and agents that modulate the expression of the hCAR gene.

### BRIEF DESCRIPTION OF THE DRAWINGS

FIGS. 1a and 1b show the results of a BLAST search using the hCAR sequence.

FIGS. 2a through 2c depict the entire cDNA sequence of the human hCAR gene with the 5′ and 3′ untranslated regions (SEQ ID NO: 1). The coding sequence is shown in uppercase starting at nucleotide 2181.

FIG. 3 depicts the nucleic acid sequence of the human hCAR coding region (SEQ ID NO: 2).

FIG. 4 depicts the amino acid sequence of the human hCAR protein (SEQ ID NO: 3).

FIGS. 5a through 5d show an alignment of the hCAR nucleic acid and protein sequence with the exon/intron boundaries indicated by vertical bars.

FIG. 6 shows the basal and forskolin stimulated cAMP levels in HEK cells transfected with pcDNA3.1+zeo/hCAR or pcDNA3.1+zeo as a control (CL).

FIGS. 7a through 7n show a 26320 bp genomic sequence which includes the hCAR gene (underlined).

FIG. 8 shows a hydrophobicity plot for hCAR. Hydrophobicity according to the GES scale (Engelman, D. M., Steitz, T. A., Goldman, A. (1986) Ann. Rev. Biophys. Chem. 15, 321-353 Identifying Nonpolar Transbilayer Helices in Amino Acid Sequences of Membrane Proteins) is plotted for the sequence of hCAR.

FIGS. 9a and 9b show alignments of ESTs from public databases with hCAR.

FIGS. 10a and 10b show alignments of ESTs from the Incyte database with hCAR.

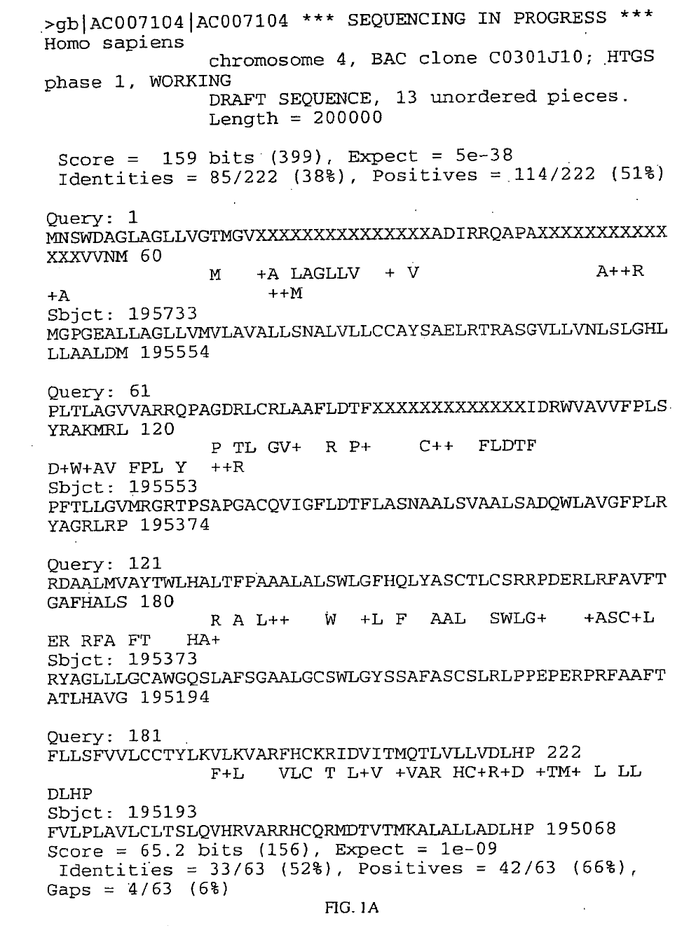

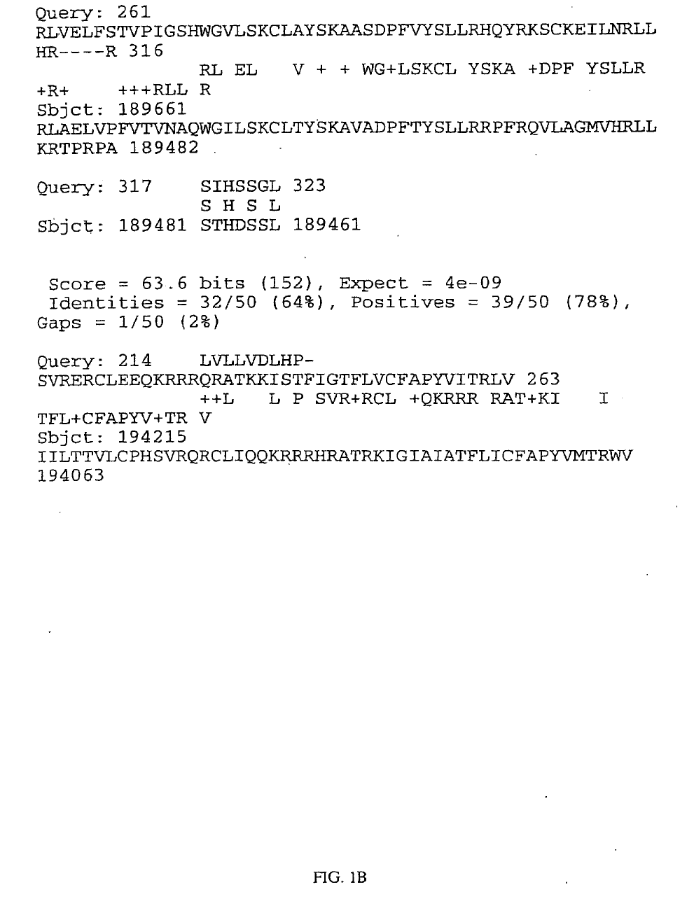

### DETAILED DESCRIPTION OF THE INVENTION

The present invention is based on the discovery of a G-protein coupled receptor (GPCR) molecule that is expressed predominantly in the brain and the placenta. The hCAR protein plays a role in signaling pathways within cells that express the hCAR protein, particularly cells of the brain and the placenta.

Various aspects of the invention are described in further detail in the following subsections:

Isolated hCAR Protein

The present invention provides isolated hCAR protein as well as peptide fragments of the hCAR protein.

Typically, hCAR is produced by recombinant expression in a non-human cell.

A hCAR protein according to the present invention encompasses a protein that comprises: 1) the amino acid sequence shown in SEQ ID NO: 2; 2) functional and non-functional naturally occurring allelic variants of human hCAR protein; 3) recombinantly produced variants of human hCAR protein; 4) hCAR proteins isolated from organisms other than humans (orthologues of human hCAR protein); and 5) useful fragments of hCAR.

An allelic variant of hCAR protein according to the present invention encompasses: 1) a protein isolated from human cells or tissues; 2) a protein encoded by the same genetic locus as that encoding the human hCAR protein; and 3) a protein that contains substantially homology to human hCAR. Examples of allelic variants may include, for example, the proteins produced by the expression of any of the single nucleotide polymorphs (SNPs) which are disclosed herein (Table 3).

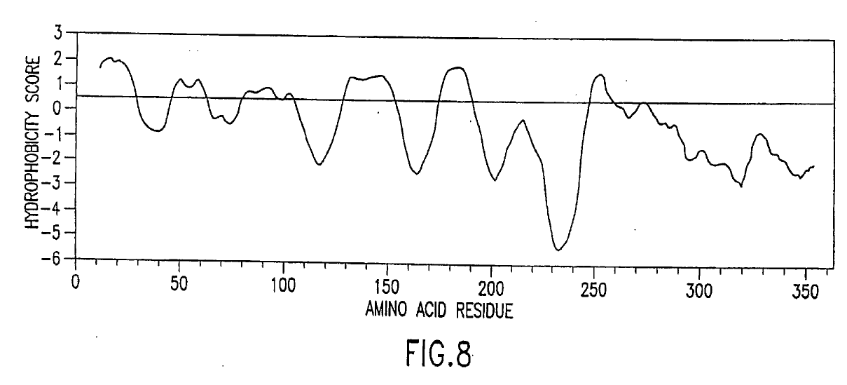

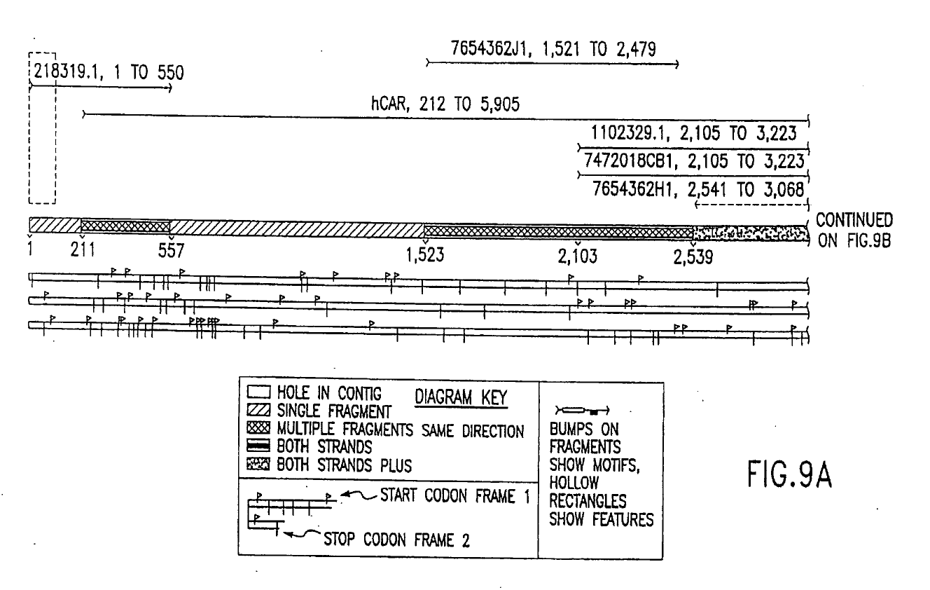

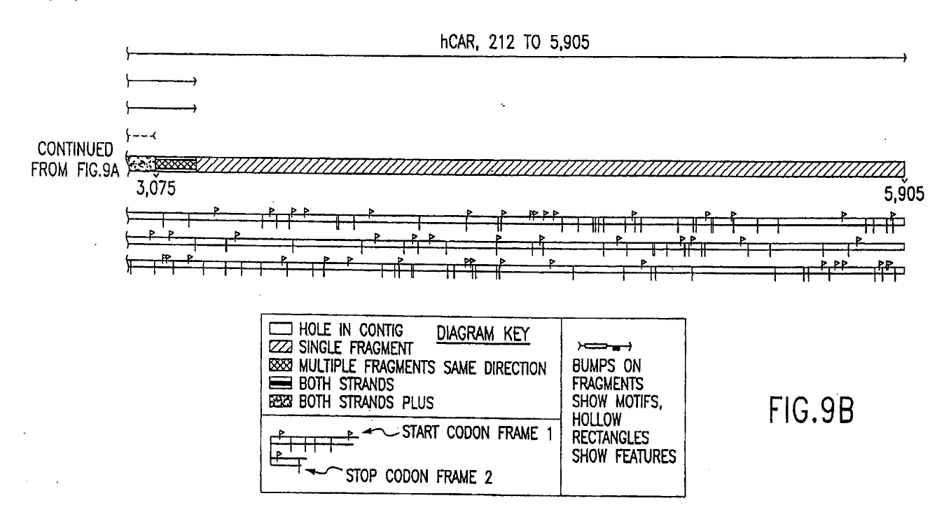

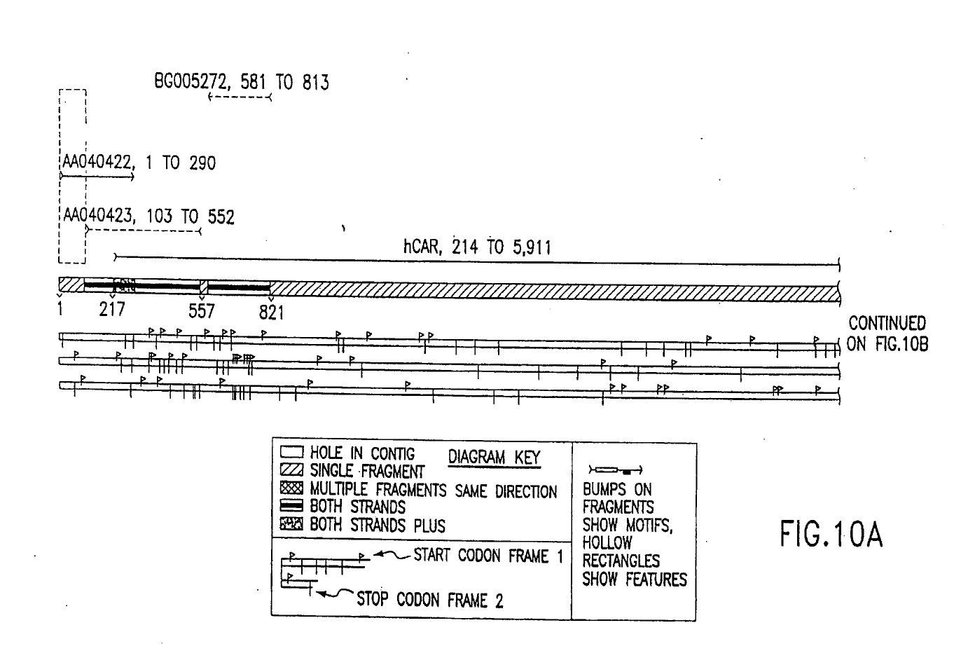

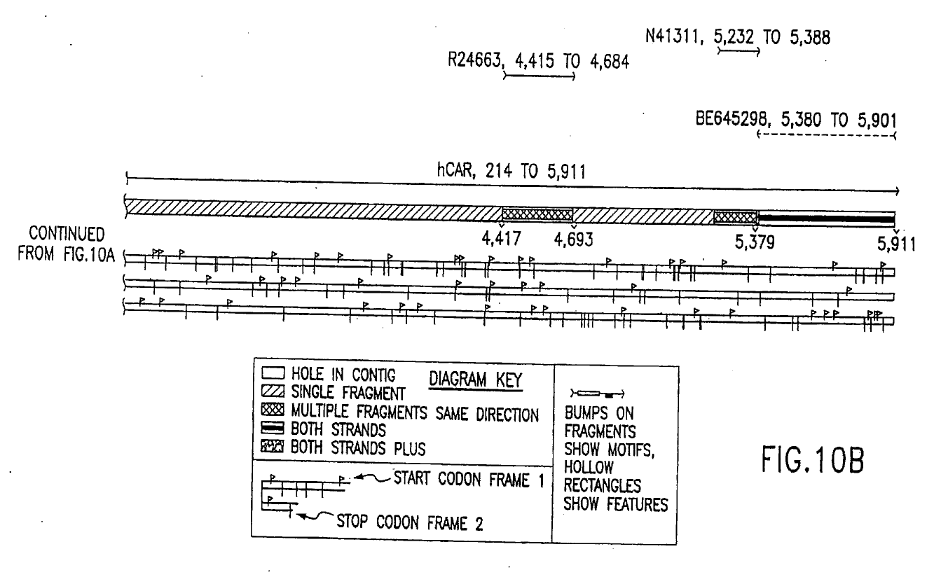

Analysis of the hydrophobicity of the hCAR protein revealed the location of the seven transmembrane regions (“TM regions”). The peak (FIG. 8) at amino acids 1-5 represents an N-terminal extracellular region. Transmembrane regions are located at amino acid positions: 6-29; 42-68; 81-102; 122-149; 174-193; 243-260; and 275-300.

As used herein, two proteins are substantially homologous when the amino acid sequence of the two proteins (or a region of the proteins) are at least about 60-65%, typically at least about 70-75%, more typically at least about 80-85%, and most typically at least about 90-95% or more homologous to each other. To determine the percent homology of two amino acid sequences (e.g., SEQ ID NO: 2 and an allelic variant thereof) or of two nucleic acids, the sequences are aligned for optimal comparison purposes (e.g., gaps can be introduced in the sequence of one protein or nucleic acid for optimal alignment with the other protein or nucleic acid). The amino acid residues or nucleotides at corresponding amino acid positions or nucleotide positions are then compared. When a position in one sequence (e.g., SEQ ID NO: 2) is occupied by the same amino acid residue or nucleotide as the corresponding position in the other sequence (e.g., an allelic variant of the human hCAR protein), then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=# of identical positions/total # of positions×100).

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35: 351-360 (1987). The method used is similar to the method described by Higgins & Sharp, CABIOS 5:151-153 (1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased.

Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff & Henikoff, Proc. Nat'l. Acad. Sci. USA 89:10915 (1989)) alignments (B) of 50, expectation (E) of 10, M=5, N=−4, and a comparison of both strands.

In addition to calculating percent sequence identity, the BLAST algorithm also performs a statistical analysis of the similarity between two sequences (see, e.g., Karlin & Altschul, Proc. Nat'l. Acad. Sci. USA 90:5873-5877 (1993)). One measure of similarity provided by the BLAST algorithm is the smallest sum probability (P(N)), which provides an indication of the probability by which a match between two nucleoticle or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Allelic variants of human hCAR include both functional and non-functional hCAR proteins. Functional allelic variants are naturally occurring amino acid sequence variants of the human hCAR protein that maintain the ability to bind an hCAR ligand and transduce a signal within a cell. Functional allelic variants will typically contain only conservative substitution of one or more amino acids of SEQ ID NO: 2 or substitution, deletion or insertion of non-critical residues in non-critical regions of the protein.

Non-functional allelic variants are naturally occurring amino acid sequence variants of human hCAR protein that do not have the ability to either bind ligand and/or transduce a signal within a cell. Non-functional allelic variants will typically contain a non-conservative substitution, a deletion, or insertion or premature truncation of the amino acid sequence of SEQ. ID. NO: 2 or a substitution, insertion or deletion in critical residues or critical regions.

The present invention further provides non-human orthologues of human hCAR protein. Orthologues of human hCAR protein are proteins that are isolated from non-human organisms and possess the same ligand binding and signaling capabilities of the human hCAR protein. Orthologues of the human hCAR protein can readily be identified as comprising an amino acid sequence that is substantially homologous to SEQ ID NO: 2.

The hCAR protein is a GPCR that participates in signaling pathways within cells. As used herein, a signaling pathway refers to the modulation (e.g., stimulated or inhibited) of a cellular function/activity upon the binding of a ligand to the GPCR (hCAR protein). Examples of such functions include mobilization of intracellular molecules that participate in a signal transduction pathway, e.g., phosphatidylinositol 4,5-bisphosphate (PIP2), inositol 1,4,5-triphosphate ON or adenylate cyclase; polarization of the plasma membrane; production or secretion of molecules; alteration in the structure of a cellular component; cell proliferation, e.g., synthesis of DNA; cell migration; cell differentiation; and cell survival. Since the hCAR protein is expressed substantially in the brain, examples of cells participating in an hCAR signaling pathway include neural cells, e.g., peripheral nervous system and central nervous system cells such as brain cells, e.g., limbic system cells, hypothalamus cells, hippocampus cells, substantia nigra cells, cortex cells, brain stem cells, neocortex cells, basal ganglion cells, caudate putamen cells, olfactory tubercle cells, and superior colliculi cells.

Depending on the type of cell, the response mediated by the hCAR protein/ligand binding may be different. For example, in some cells, binding of a ligand to an hCAR protein may stimulate an activity such as adhesion, migration, differentiation, etc. through phosphatidylinositol or cyclic AMP metabolism and turnover while in other cells, the binding of the ligand to the hCAR protein will produce a different result. Regardless of the cellular activity modulated by hCAR, it is universal that the hCAR protein is a GPCR and interacts with a “G protein” to produce one or more secondary signals in a variety of intracellular signal transduction pathways, e.g., through phosphatidylinositol or cyclic AMP metabolism and turnover, in a cell. G proteins represent a family of heterotrimeric proteins composed of α, β and γ subunits, which bind guanine nucleotides. These proteins are usually linked to cell surface receptors, e.g., receptors containing seven transmembrane domains, such as the ligand receptors. Following ligand binding to the receptor, a conformational change is transmitted to the G protein, which causes the α-subunit to exchange a bound GDP molecule for a GTP molecule and to dissociate from the N-subunits. The GTP-bound form of the α-subunit typically functions as an effector-modulating moiety, leading to the production of second messengers, such as cyclic AMP (e.g., by activation of adenylate cyclase), diacylglycerol or inositol phosphates. Greater than 20 different types of α-subunits are known in man, which associate with a smaller pool of β and γ subunits.

A signaling pathway in which the hCAR protein may participate is the cAMP turnover pathway. As used herein, “cyclic AMP turnover and metabolism” refers to the molecules involved in the turnover and metabolism of cyclic AMP (cAMP) as well as to the activities of these molecules. Cyclic AMP is a second messenger produced in response to ligand induced stimulation of certain G protein coupled receptors. In the ligand signaling pathway, binding of ligand to a ligand receptor can lead to the activation of the enzyme adenylate cyclase, which catalyzes the synthesis of cAMP. The newly synthesized cAMP can in turn activate a cAMP-dependent protein kinase. This activated kinase can phosphorylate a voltage-gated potassium channel protein, or an associated protein, and lead to the inability of the potassium channel to open during an action potential. The inability of the potassium channel to open results in a decrease in the outward flow of potassium, which normally repolarizes the membrane of a neuron, leading to prolonged membrane depolarization.

The present invention further provides fragments of hCAR proteins. As used herein, a fragment comprises at least 3 contiguous amino acids from an hCAR protein.

Preferred fragments are fragments that possess one or more of the biological activities of the hCAR protein, for example the ability to bind to a G-protein, as well as fragments that can be used as an immunogen to generate anti-hCAR antibodies. Biologically active fragments of the hCAR protein include peptides comprising amino acid sequences derived from the amino acid sequence of an hCAR protein, e.g., the amino acid sequence shown in SEQ ID NO: 2 or the amino acid sequence of a protein homologous to the hCAR protein, which include less amino acids than the full length hCAR protein or the full length protein which is homologous to the hCAR protein, and exhibit at least one activity of the hCAR protein. Typically, biologically active fragments (peptides, e.g., peptides which are, for example, 5, 10, 15, 20, 30, 35, 36, 37, 38, 39, 40, 50, 100 or more amino acids in length) comprise a domain or motif, e.g., a transmembrane domain or G-protein binding domain. Representative fragments include the extracellular domain peptides of SEQ ID NOs: 4, 5, 6 and 7.

Modifications and changes can be made in the structure of a polypeptide of the present invention and still obtain a molecule having GPCR like receptor characteristics. For example, certain amino acids can be substituted for other amino acids in a sequence without appreciable loss of receptor activity. Because it is the interactive capacity and nature of a polypeptide that defines that polypeptide's biological functional activity, certain amino acid sequence substitutions can be made in a polypeptide sequence (or, of course, its underlying DNA coding sequence) and nevertheless obtain a polypeptide according to the present invention.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The relative hydropathic character of the amino acid residue determines the secondary and tertiary structure of the resultant polypeptide, which in turn defines the interaction of the polypeptide with other molecules, such as enzymes, substrates, receptors, antibodies, antigens, and the like. It is known in the art that an amino acid may be substituted by another amino acid having a similar hydropathic index and still obtain a functionally equivalent polypeptide. In such changes, the substitution of amino acids whose hydropathic indices are within +/−2 is preferred, those which are within +/−1 are particularly preferred, and those within +/−0.5 are even more particularly preferred.

Substitution of like amino acids can also be made on the basis of hydrophilicity, particularly where the biological functional equivalent polypeptide or peptide thereby created is intended for use in immunological embodiments. U.S. Pat. No. 4,554,101, incorporated herein by reference, states that the greatest local average hydrophilicity of a polypeptide, as governed by the hydrophilicity of its adjacent amino acids, correlates with its immunogenicity and antigenicity, i.e. with a biological property of the polypeptide.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those which are within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine (See Table 1, below). The present invention thus contemplates functional or biological equivalents of a GPCR polypeptide as set forth above.

TABLE 1

| Original Residue | Exemplary Residue Substitution |
|---|---|
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Met; Leu; Tyr |
| Ser | Thr |
| Thr | Ser |
| Trp | Tyr |
| Tyr | Trp; Phe |
| Val | Ile; Leu |

Biological or functional equivalents of a polypeptide can also be prepared using site-specific mutagenesis. Site-specific mutagenesis is a technique useful in the preparation of second generation polypeptides, or biologically functional equivalent polypeptides or peptides, derived from the sequences thereof, through specific mutagenesis of the underlying DNA. As noted above, such changes can be desirable where amino acid substitutions are desirable. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

In general, the technique of site-specific mutagenesis is well known in the art. As will be appreciated, the technique typically employs a phage vector which can exist in both a single stranded and double stranded form. Typically, site-directed mutagenesis in accordance herewith is performed by first obtaining a single-stranded vector which includes within its sequence a DNA sequence which encodes all or a portion of the GPCR polypeptide sequence selected. An oligonucleotide primer bearing the desired mutated sequence is prepared (e.g., synthetically). This primer is then annealed to the singled-stranded vector, and extended by the use of enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells such as E. coli cells and clones are selected which include recombinant vectors bearing the mutation. Commercially available kits come with all the reagents necessary to perform site directed metagenesis, except the oligonucleotide primers.

A hCAR receptor polypeptide of the present invention is understood to be any hCAR polypeptide comprising substantial sequence similarity, structural similarity and/or functional similarity to a hCAR polypeptide comprising the amino acid sequence of SEQ ID NO: 2. In addition, a hCAR polypeptide of the invention is not limited to a particular source. Thus, the invention provides for the general detection and isolation of the genus of hCAR receptor polypeptides from a variety of sources. For example hCAR polypeptides are found in virtually all mammals including human. As is the case with other receptors, there is likely little variation between the structure and function of hCAR receptors in different species. Where there is a difference between species, identification of those differences is well within the skill of an artisan. Thus, the present invention contemplates a hCAR polypeptide from any animal, wherein the preferred animal is a mammal and the preferred mammal is a human.

It is contemplated in the present invention, that a hCAR may advantageously be cleaved into fragments for use in further structural or functional analysis, or in the generation of reagents such as hCAR-related polypeptides and hCAR-specific antibodies. This can be accomplished by treating purified or unpurified hCAR with a peptidase such as endoproteinase glu-C (Boehringer, Indianapolis, Ind.). Treatment with CNBr is another method by which hCAR fragments may be produced from natural hCAR. Recombinant techniques also can be used to produce specific fragments of hCAR.

In addition, the inventors also contemplate that compounds sterically similar to a hCAR may be formulated to mimic the key portions of the peptide structure, called peptidomimetics. Mimetics are peptide-containing molecules which mimic elements of protein secondary structure. See, for example, Johnson et al. (1993). The underlying rationale behind the use of peptide mimetics is that the peptide backbone of proteins exists chiefly to orient amino acid side chains in such a way as to facilitate molecular interactions, such as those of receptor and ligand.

Successful applications of the peptide mimetic concept have thus far focused on mimetics of β-turns within proteins. Likely β-turn structures within GPCR can be predicted by computer-based algorithms as discussed above. Once the component amino acids of the turn are determined, mimetics can be constructed to achieve a similar spatial orientation of the essential elements of the amino acid side chains, as discussed in Johnson et al. (1993).

The isolated hCAR proteins can be purified from cells that naturally express the protein, purified from cells that have been altered to express the hCAR protein, or synthesized using known protein synthesis methods. Preferably, as described below, the isolated hCAR protein is produced by recombinant DNA techniques. For example, a nucleic acid molecule encoding the protein is cloned into an expression vector, the expression vector is introduced into a host cell and the hCAR protein is expressed in the host cell. The hCAR protein can then be isolated from the cells by an appropriate purification scheme using standard protein purification techniques. As an alternative to recombinant expression, the hCAR protein or fragment can be synthesized chemically using standard peptide synthesis techniques. Lastly, native hCAR protein can be isolated from cells that naturally express the hCAR protein (e.g., hippocampal cells, or substantia nigra cells). The present invention further provides hCAR chimeric or fusion proteins. As used herein, an hCAR “chimeric protein” or “fusion protein” comprises an hCAR protein operatively linked to a non-hCAR protein. An “hCAR protein” refers to a protein having an amino acid sequence corresponding to an hCAR protein, whereas a “non-hCAR protein” refers to a heterologous protein having an amino acid sequence corresponding to a protein which is not substantially homologous to the hCAR protein, e.g., a protein which is different from the hCAR protein. Within the context of fusion proteins, the term “operatively linked” is intended to indicate that the hCAR protein and the non-hCAR protein are fused in-frame to each other. The non-hCAR protein can be fused to the N-terminus or C-terminus of the hCAR protein. For example, in one embodiment the fusion protein is a GST-hCAR fusion protein in which the hCAR sequences are fused to the C-terminus of the GST sequences. Other types of fusion proteins include, but are not limited to, enzymatic fusion proteins, for example beta-galactosidase fusions, yeast two-hybrid GAL fusions, poly-His fusions and Ig fusions.

Such fusion proteins, particularly poly-His fusions, can facilitate the purification of recombinant hCAR protein. In another embodiment, the fusion protein is an hCAR protein containing a heterologous signal sequence at its N-terminus. In certain host cells (e.g., mammalian host cells), expression and/or secretion of an hCAR protein can be increased by using a heterologous signal sequence.

Preferably, an hCAR chimeric or fusion protein is produced by standard recombinant DNA techniques. For example, DNA fragments coding for the different protein sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST protein). An hCAR-encoding nucleic acid can be cloned into such an expression vector such that the fusion moiety is linked in-frame to the hCAR protein.

Antibodies that Bind to an hCAR Protein

The present invention further provides antibodies that selectively bind to a hCAR protein. As used herein, an antibody is said to selectively bind to an hCAR protein when the antibody binds to hCAR proteins and does not selectively bind to unrelated proteins. A skilled artisan will readily recognize that an antibody may be considered to substantially bind an hCAR protein even if it binds to proteins that share homology with a fragment or domain of the hCAR protein.

The term “antibody” as used herein refers to immunoglobulin molecules and immunologically active fragments of immunoglobulin molecules, i.e., molecules that contain an antigen binding site which specifically binds (immunoreacts with) an antigen, such as hCAR. Examples of immunologically active fragments of immunoglobulin molecules include F(ab) and F(ab′)2 fragments which can be generated by treating the antibody with an enzyme such as pepsin. The invention provides polyclonal and monoclonal antibodies that bind hCAR. The term “monoclonal antibody” or “monoclonal antibody composition,” as used herein, refers to a population of antibody molecules that contain only one species of an antigen binding site capable of immunoreacting with a particular epitope of hCAR. A monoclonal antibody composition thus typically displays a single binding affinity for a particular hCAR protein with which it immunoreacts.

To generate anti-hCAR antibodies, an isolated hCAR protein, or a fragment thereof, is used as an immunogen to generate antibodies that bind hCAR using standard techniques for polyclonal and monoclonal antibody preparation. The full-length hCAR protein can be used or, alternatively, an antigenic peptide fragment of hCAR can be used as an immunogen. An antigenic fragment of the hCAR protein will typically comprises at least 3 contiguous amino acid residues of an hCAR protein, e.g. 3 contiguous amino acids from SEQ ID NO: 2. Preferably, the antigenic peptide comprises at least 5 amino acid residues. Preferred fragments for generating anti-hCAR antibodies are regions of hCAR that are located on the surface of the protein (extracellular regions) as exemplified in Example 16.

An hCAR immunogen typically is used to prepare antibodies by immunizing a suitable subject, (e.g., rabbit, goat, mouse or other mammal, chicken) with the immunogen. An appropriate immunogenic preparation can contain, for example, recombinantly expressed hCAR protein or a chemically synthesized hCAR peptide. The preparation can further include an adjuvant, such as Freund's complete or incomplete adjuvant, or similar immunostimulatory agent. Immunization of a suitable subject with an immunogenic hCAR preparation induces a polyclonal anti-hCAR antibody response.

Polyclonal anti-hCAR antibodies can be prepared as described above by immunizing a suitable subject with an hCAR immunogen. The anti-hCAR antibody titer in the immunized subject can be monitored over time by standard techniques, such as with an enzyme linked immunosorbent assay (ELISA) using immobilized hCAR. If desired, the antibody molecules directed against hCAR can be isolated from the mammal (e.g., from the blood) and further purified by well known techniques, such as protein A chromatography to obtain the IgG fraction. At an appropriate time after immunization, e.g., when the anti-hCAR antibody titers are highest, antibody-producing cells can be obtained from the subject and used to prepare monoclonal antibodies by standard techniques, such by using hybridoma technique.

The more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known. Briefly, an immortal cell line (typically a myeloma) is fused to lymphocytes (typically splenocytes) from a mammal immunized with an hCAR immunogen as described above, and the culture supernatants of the resulting hybridoma cells are screened to identify a hybridoma producing a monoclonal antibody that binds hCAR.

Any of the many well known protocols used for fusing lymphocytes and immortalized cell lines can be applied for the purpose of generating an anti-hCAR monoclonal antibody. Moreover, the ordinarily skilled worker will appreciate that there are many variations of such methods which also would be useful.

Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NSI/1-Ag4-1, P3-x63-Ag8.653 or Sp2/0-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind hCAR, e.g., using a standard ELISA assay. Alternative to preparing monoclonal antibody-secreting hybridomas, a monoclonal anti-hCAR antibody can be identified and isolated by screening a recombinant combinatorial immunoglobulin library (e.g., an antibody phage display library) with hCAR to thereby isolate immunoglobulin library members that bind hCAR. Kits for generating and screening phage display libraries are commercially available (e.g., the Pharmacia Recombinant Phage Antibody System, Catalog No. 27-9400-0 1; and the Stratagene SurJZ4pTM Phage Display Kit, Catalog No. 240612).

Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809.

Additionally, recombinant anti-hCAR antibodies, such as chimeric and humanized monoclonal antibodies, comprising both human and non-human fragments, which can be made using standard recombinant DNA techniques, are within the scope of the invention. Such chimeric and humanized monoclonal antibodies can be produced by recombinant DNA techniques known in the art, for example using methods described in Robinson et al. PCT International Application No. PCT/US86/02269; Akira, et al. European Patent Application 1174148; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023.

An anti-hCAR antibody (e.g., monoclonal antibody) can be used to isolate hCAR proteins by standard techniques, such as affinity chromatography or immunoprecipitation. An anti-hCAR antibody can facilitate the purification of natural hCAR protein from cells and recombinantly produced hCAR protein expressed in host cells. Moreover, an anti-hCAR antibody can be used to detect hCAR protein (e.g., in a cellular lysate or cell supernatant) in order to evaluate the abundance and pattern of expression of the hCAR protein. The detection of circulating fragments of an hCAR protein can be used to identify hCAR protein turnover in a subject. Anti-hCAR antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, P-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and acquorin, and examples of suitable radioactive material include 125_{I}, 131_{I}, 15_{S} or 3_{H}.

Particularly useful antibodies of the present invention include those that specifically bind to the extracellular regions as determined by the structural and hydrophobicity analysis of hCAR (see Example 16, infra). Such regions include those at amino acid positions 1-5, 69-80, 150-173, and 261-274. Such antibodies can be manufactured against the entire hCAR protein or against isolated peptides which comprise the extracellular regions. Such peptides include: Met Gly Pro Gly Glu (SEQ ID NO: 4);

(SEQ ID NO: 5)

Arg Gly Arg Thr Pro Ser Ala Pro Gly Ala Cys Gln;

(SEQ ID NO: 6)

Ser Ser Ala Phe Ala Ser Cys Ser Leu Arg Leu Pro
Pro Glu Pro Glu Arg Pro Arg Phe Ala Ala Phe Thr;
and

(SEQ ID NO: 7)

Arg Leu Ala Glu Leu Val Pro Phe Val Thr Val Asn
Ala Gln.

Isolated hCAR Nucleic Acid Molecules

The present invention further provides isolated nucleic acid molecules that encode an hCAR protein, hereinafter the hCAR gene or hCAR nucleic acid molecule, as well as fragments of a hCAR gene.

As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded.

The isolated nucleic acid molecules of the present invention encode an hCAR protein. As described above, an hCAR protein is defined as a protein comprising the amino acid sequence depicted in SEQ ID NO: 2 (human hCAR protein), allelic variants of human hCAR protein, and orthologues of the human hCAR protein. A preferred hCAR nucleic acid molecule comprises the nucleotide sequence shown in SEQ ID NO: 1. The sequence of SEQ ID NO: 1 corresponds to the human hCAR cDNA. This cDNA comprises sequences encoding the human hCAR protein (i.e., “the coding region,” from nucleotides 1892 to 2980 of SEQ ID NO: 1), as well as 5′ untranslated sequences (nucleotides 1 to 1891 of SEQ ID NO: 1) and 3′ untranslated sequences (nucleotides 2981 to 5665 of SEQ ID NO: 1).

Alternatively, the nucleic acid molecule can comprise only the coding region of SEQ ID NO: 1 (e.g., nucleotides 1892 to 2980 of SEQ ID NO: 1). The human hCAR gene is approximately 26320 nucleotides in length and encodes a full length protein having a molecular weight of approximately 39 KDa and which is 363 amino acid residues in length. The human hCAR protein is expressed primarily in the brain and the placenta, particularly the cerebral cortex, frontal lobe, parietal lobe, occipital lobe, temporal lobe, paracentral gyrus of cerebral cortex, pons, left and right cerebellum, corpus callosum, amygdala, caudate nucleus, hippocampus, medulla oblongata, putamen, substantia nigra, accumbens nucleus, thalamus, pituitary gland and spinal cord. Based on structural analysis, see Example 16, amino acid positions: 6-29; 42-68; 81-102; 122-149; 174-193; 243-260; and 275-300 comprise transmembrane domains. As used herein, the term “transmembrane domain” refers to a structural amino acid motif which includes a hydrophobic helix that spans the plasma membrane.

The invention further encompasses nucleic acid molecules that differ from the nucleotide sequence shown in SEQ ID NO: 1 (and fragments thereof) due to degeneracy of the genetic code and thus encode the same hCAR protein as that encoded by the nucleotide sequence shown in SEQ ID NO: 1.

In another preferred embodiment, an isolated nucleic acid molecule of the invention comprises a nucleic acid molecule which is a complement of the nucleotide sequence shown in SEQ ID NO: 1, or a fragment of this nucleotide sequences. A nucleic acid molecule which is complementary to the nucleotide sequence shown in SEQ ID NO: 1 is one which is sufficiently complementary to the nucleotide sequence shown in SEQ ID NO: 1 such that it can hybridize to the nucleotide sequence shown in SEQ ID NO: 1, thereby forming a stable duplex.

Orthologues and allelic variants of the human hCAR gene can readily be identified using methods well known in the art. Allelic variants and orthologues of the human hCAR gene will comprise a nucleotide sequence that is typically at least about 70-75%, more typically at least about 80-85%, and most typically at least about 90-95% or more homologous to the nucleotide sequence shown in SEQ ID NO: 1, or a fragment of these nucleotide sequences. Such nucleic acid molecules can readily be identified as being able to hybridize, preferably under stringent conditions, to the nucleotide sequence shown in SEQ ID NO: 1, or a fragment of either of this nucleotide sequence.

Moreover, the nucleic acid molecule of the invention can comprise only a fragment of the coding region of a hCAR gene, such as a fragment of SEQ ID NO: 1.

The nucleotide sequence determined from the cloning of the human hCAR gene allows for the generation of probes and primers designed for use in identifying and/or cloning hCAR gene homologues from other cell types, e.g., from other tissues, as well as hCAR gene orthologues from other mammals. A probe/primer typically comprises substantially purified oligonucleotide. The oligonucleotide typically comprises a region of nucleotide sequence that hybridizes under stringent conditions to at least about 12, preferably about 25, more preferably about 40, 50 or 75 consecutive nucleotides of SEQ ID NO: 1 sense, an anti-sense sequence of SEQ ID NO: 1, or naturally occurring mutants thereof. Primers based on the nucleotide sequence in SEQ ID NO: 1 can be used in PCR reactions to clone hCAR gene homologues. Probes based on the hCAR nucleotide sequence can be used to detect transcripts or genomic sequences encoding the same or homologous proteins. In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress an hCAR protein, such as by measuring a level of an hCAR-encoding nucleic acid in a sample of cells from a subject e.g., detecting hCAR mRNA levels or determining whether a genomic hCAR gene has been mutated or deleted.

In addition to the hCAR nucleotide sequence shown in SEQ ID NO: 1, it will be appreciated by those skilled in the art that DNA sequence polymorphisms that lead to changes in the amino acid sequences of an hCAR protein may exist within a population (e.g., the human population). Such genetic polymorphism in the hCAR gene may exist among individuals within a population due to natural allelic variation. Such natural allelic variations can typically result in 1-5% variance in the nucleotide sequence of the hCAR gene. Any and all such nucleotide variations and resulting amino acid polymorphisms in a hCAR gene that are the result of natural allelic variation are intended to be within the scope of the invention. Such allelic variation includes both active allelic variants as well as non-active or reduced activity allelic variants, the later two types typically giving rise to a pathological disorder. Polymorphisms of hCAR are disclosed in Example 15.

Moreover, nucleic acid molecules encoding hCAR proteins from other species, and thus which have a nucleotide sequence which differs from the human sequence of SEQ ID NO: 1, are intended to be within the scope of the invention. Nucleic acid molecules corresponding to natural allelic variants and non-human orthologues of the human hCAR cDNA of the invention can be isolated based on their homology to the human hCAR nucleic acid disclosed herein using the human cDNA, or a fragment thereof, as a hybridization probe according to standard hybridization techniques under stringent hybridization conditions.

Accordingly, in another embodiment, an isolated nucleic acid molecule of the invention is at least 15 nucleotides in length and hybridizes under stringent conditions to the nucleic acid molecule comprising the nucleotide sequence of SEQ ID NO: 1. In other embodiments, the nucleic acid is at least 30, 50, 100, 250 or 500 nucleotides in length.

In accordance with the present invention, nucleotide sequences which encode hCAR, fragments, fusion proteins or functional equivalents thereof, may be used to generate recombinant DNA molecules that direct the expression of hCAR, or a functionally active peptide, in appropriate host cells. Alternatively, nucleotide sequences which hybridize to portions of the hCAR sequence may be used in nucleic acid hybridization assays, Southern and Northern blot assays, etc.

The invention also includes polynucleotides with sequences complementary to those of the polynucleotides disclosed herein.

The present invention also includes polynucleotides capable of hybridizing under reduced stringency conditions, more preferably stringent conditions, and most preferably highly stringent conditions, to polynucleotides described herein. Examples of stringency conditions are shown in the table below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 2
Stringency Conditions

| | Poly- | Hybrid | Hybridization | Wash |
|---|---|---|---|---|
| Stringency | nucleotide | Length | Temperature and | Temperature |
| Condition | Hybrid | (bp)¹ | Buffer^{H} | and Buffer^{H} |
| A | DNA:DNA | >50 | 65EC; 1xSSC | 65EC; |
| | | | -or- 42EC; 1xSSC, | 0.3xSSC |
| | | | 50% formamide | |
| B | DNA:DNA | <50 | T_{B}*; 1xSSC | T_{B}*; |
| | | | | 1xSSC |
| C | DNA:RNA | >50 | 67EC; 1xSSC | 67EC; |
| | | | -or- 45EC; 1xSSC, | 0.3xSSC |
| | | | 50% formamide | |
| D | DNA:RNA | <50 | T_{D}*; 1xSSC | T_{D}*; |
| | | | | 1xSSC |
| E | RNA:RNA | $50 | 70EC; 1xSSC | 70EC; |
| | | | -or- 50EC; 1xSSC, | 0.3xSSC |
| | | | 50% formamide | |
| F | RNA:RNA | <50 | T_{F}*; 1xSSC | T_{f}*; |
| | | | | 1xSSC |
| G | DNA:DNA | >50 | 65EC; 4xSSC | 65EC; |
| | | | -or- 42EC; 4xSSC, | 1xSSC |
| | | | 50% formamide | |
| H | DNA:DNA | <50 | T_{H}*; 4xSSC | T_{H}*; |
| | | | | 4xSSC |
| I | DNA:RNA | >50 | 67EC; 4xSSC | 67EC; |
| | | | -or- 45EC; 4xSSC, | 1xSSC |
| | | | 50% formamide | |
| J | DNA:RNA | <50 | T_{J}*; 4xSSC | T_{J}*; |
| | | | | 4xSSC |
| K | RNA:RNA | >50 | 70EC; 4xSSC | 67EC; |
| | | | -or- 50EC; 4xSSC, | 1xSSC |
| | | | 50% formamide | |
| L | RNA:RNA | <50 | T_{L}*; 2xSSC | T_{L}*; |
| | | | | 2xSSC |
| M | DNA:DNA | >50 | 50EC; 4xSSC | 50EC; |
| | | | -or- 40EC; 6xSSC, | 2xSSC |
| | | | 50% formamide | |
| N | DNA:DNA | <50 | T_{N}*; 6xSSC | T_{N}*; |
| | | | | 6xSSC |
| O | DNA:RNA | >50 | 55EC; 4xSSC | 55EC; |
| | | | -or- 42EC; 6xSSC, | 2xSSC |
| | | | 50% formamide | |
| P | DNA:RNA | <50 | T_{P}*; 6xSSC | T_{P}*; |
| | | | | 6xSSC |
| Q | RNA:RNA | >50 | 60EC; 4xSSC | 60EC; |
| | | | -or- 45EC; 6xSSC, | 2xSSC |
| | | | 50% formamide | |
| R | RNA:RNA | <50 | T_{R}*; 4xSSC | T_{R}*; |
| | | | | 4xSSC |

¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity.
^{H}SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers; washes are performed for 15 minutes after hybridization is complete.
T_{B}*-T_{R}*: The hybridization temperature for hybrids anticipated to be less than 50 base pairs in length should be 5-10EC less than the melting temperature (Tₘ) of the hybrid, where Tₘ is determined according to the following equations. For hybrids less than 18 base pairs in length, Tₘ(EC) = 2(# of A + T bases) + 4(# of G + C bases). For hybrids between 18 and 49 base pairs in length, Tₘ(EC) = 81.5 + 16.6(log₁₀Na⁺) + 0.41 (% G + C) − (600/N), where N is the number of bases in the hybrid, and Na⁺ is the concentration of sodium ions in the hybridization buffer (Na⁺ for 1 × SSC = 0.165 M).

Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

Preferably, each such hybridizing polynucleotide has a length that is at least 25% (more preferably at least 50%, and most preferably at least 75%) of the length of the polynucleotide of the present invention to which it hybridizes, and has at least 60% sequence identity (more preferably, at least 75% identity; most preferably at least 90% or 95% identity) with the polynucleotide of the present invention to which it hybridizes, where sequence identity is determined by comparing the sequences of the hybridizing polynucleotides when aligned so as to maximize overlap and identity while minimizing sequence gaps.

In addition to naturally-occurring allelic variants of the hCAR nucleic acid sequence that may exist in the population, the skilled artisan will further appreciate that changes can be introduced by mutation into the nucleotide sequence of SEQ ID NO: 1 as described above.

Accordingly, another aspect of the invention pertains to nucleic acid molecules encoding hCAR proteins that contain changes in amino acid residues that are not essential for hCAR activity. Such hCAR proteins differ in amino acid sequence from SEQ ID NO: 2 yet retain at least one of the hCAR activities described herein. In one embodiment, the isolated nucleic acid molecule comprises a nucleotide sequence encoding a protein, wherein the protein comprises an amino acid sequence at least about 30-35%, preferably at least about 40-45%, more preferably at least about 50-55%, even more preferably at least about 60-65%, yet more preferably at least about 70-75%, still more preferably at least about 80-85%, and most preferably at least about 90-95% or more homologous to the amino acid sequence of SEQ ID NO: 2.

In another embodiment, mutations can be introduced randomly along all or part of a hCAR coding sequence, such as by saturation mutagenesis, and the resultant mutants can be screened for an hCAR activity described herein to identify mutants that retain hCAR activity. Following mutagenesis of SEQ ID NO: 1, the encoded protein can be expressed recombinantly and the activity of the protein can be determined using, for example, assays described herein.

In addition to the nucleic acid molecules encoding hCAR proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire hCAR coding strand, or to only a fragment thereof. In one embodiment, an antisense nucleic acid molecule is antisense to a “coding region” of the coding strand of a nucleotide sequence encoding an hCAR protein.

The term “coding region” refers to the region of the nucleotide sequence comprising codons which are translated into amino acid residues, e.g., the entire coding region of SEQ ID NO: 1 comprises nucleotides 1892 to 2983. In another embodiment, the antisense nucleic acid molecule is antisense to a “noncoding region” of the coding strand of a nucleotide sequence encoding an hCAR protein. The term “noncoding region” refers to 5′ and 3′ sequences which flank the coding region that are not translated into amino acids (i.e., also referred to as 5′ and 3′ untranslated regions).

Given the coding strand sequence encoding the hCAR protein disclosed herein (e.g., SEQ ID NO: 1), antisense nucleic acids of the invention can be designed according to the rules of Watson and Crick base pairing. The antisense nucleic acid molecule can be complementary to the entire coding region of hCAR mRNA, but more preferably is an oligonucleotide which is antisense to only a fragment of the coding or noncoding region of hCAR mRNA. For example, the antisense oligonucleotide can be complementary to the region surrounding the translation start site of hCAR mRNA.

An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an hCAR protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of an antisense nucleic acid molecule of the invention includes direct injection at a tissue site. Alternatively, an antisense nucleic acid molecule can be modified to target selected cells and then administered systemically. For example, for systemic administration, an antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein.

In yet another embodiment, the antisense nucleic acid molecule of the invention is an α-anomeric nucleic acid molecule. An μ-anomeric nucleic acid molecule forms specific double-stranded hybrids with complementary RNA in which, contrary to the usual γ-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave hCAR mRNA transcripts to thereby inhibit translation of hCAR mRNA. A ribozyme having specificity for an hCAR-encoding nucleic acid can be designed based upon the nucleotide sequence of an hCAR gene disclosed herein (i.e., SEQ ID NO: 1). For example, a derivative of a Tetrahymena L-19 IVS RNA can be constructed in which the nucleotide sequence of the active site is complementary to the nucleotide sequence to be cleaved in an hCAR-encoding mRNA. See, e.g., Cech et al. U.S. Pat. No. 4,987,071 and Cech et al. U.S. Pat. No. 5,116,742 both incorporated by reference. Alternatively, hCAR mRNA can be used to select a catalytic RNA having a specific ribonuclease activity from a pool of RNA molecules. See, e.g., Bartel, D. and Szostak, J. W. (1993) Science 261:1411-1418.

Alternatively hCAR gene expression can be inhibited by targeting nucleotide sequences complementary to the regulatory region of the hCAR gene (e.g., the hCAR gene promoter and/or enhancers) to form triple helical structures that prevent transcription of the hCAR gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

hCAR gene expression can also be inhibited using RNA interference (RNAi). This is a technique for post-transcriptional gene silencing (PTGS), in which target gene activity is specifically abolished with cognate double-stranded RNA (dsRNA). RNAi resembles in many aspects PTGS in plants and has been detected in many invertebrates including trypanosome, hydra, planaria, nematode and fruit fly (Drosophila melanogaster). It may be involved in the modulation of transposable element mobilization and antiviral state formation. RNAi in mammalian systems is disclosed in PCT application WO 00/63364 which is incorporated by reference herein in its entirety. Basically, dsRNA of at least about 600 nucleotides, homologous to any portion of the target (hCAR) is introduced into the cell by microinjection or transfection of dsRNA that has been synthesized in vitro or by introduction into the cell of a transgene that encodes a target RNA transcript that can foldback to yield a dsRNA and a sequence specific reduction in gene activity is observed.

### Recombinant Expression Vectors, Host Cells, Transgenics, and Knockouts

Another aspect of the invention pertains to vectors, preferably expression vectors, containing a nucleic acid encoding an hCAR protein (or a fragment thereof).

As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby. are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences) or at certain points in development. It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., hCAR proteins, altered forms of hCAR proteins, fusion proteins, and the like).

The recombinant expression vectors of the invention can be designed for expression of an hCAR protein in prokaryotic or eukaryotic cells. For example, an hCAR protein can be expressed in bacterial cells such as E. coli, insect cells (e.g., using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, either to the amino or carboxyl terminus. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Typical fusion expression vectors include pGEX (Pharmacia Biotech Inc; Smith, D. B. and Johnson, K. S. (1988) Gene 67:31-40), pMAL (New England Biolabs, Beverly; MA), pRIT5 (Pharmacia, Piscataway, N.J.) which fuse glutathione S-transferase (GST), maltose E binding protein, or protein A, respectively, to the target recombinant protein, and pcDNA3.1 (Invitrogen Corporation, Carlsbad, Calif.).

In one embodiment, the coding sequence of the hCAR gene is cloned into a pGEX expression vector to create a vector encoding a fusion protein comprising, from the N-terminus to the C-terminus, GST-thrombin cleavage site-hCAR protein. The fusion protein can be purified by affinity chromatography using glutathione-agarose resin.

Recombinant hCAR protein unfused to GST can be recovered by cleavage of the fusion protein with thrombin.

Examples of suitable inducible non-fusion E. coli expression vectors include pTrc (Amann et al., (1988) Gene 69:301-315) and pET I I d (Studier et al., Gene Expression Technology Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET I I d vector relies on transcription from a T7 gn1 0-lac fusion promoter mediated by a coexpressed viral RNA polymerase J7 gnl). This viral polymerase is supplied by host strains BL21 (DE3) or HMS I 74(DE3) from a resident prophage harboring a T7 gnl gene under the transcriptional control of the lacUV 5 promoter.

One strategy to maximize recombinant protein expression in E. coli is to express the protein in a host bacteria with an impaired capacity to proteolytically cleave the recombinant protein. Another strategy is to alter the nucleic acid sequence of the nucleic acid to be inserted into an expression vector so that the individual codons for each amino acid are those preferentially utilized in E. coli.

Such alteration of nucleic acid sequences of the invention can be carried out by standard DNA mutagenesis or synthesis techniques.

In another embodiment, the hCAR gene expression vector is a yeast expression vector. Examples of vectors for expression in yeast S. cerivisae include pYepSec I (Baldari, et al., (1987) Embo J 6:229-234), pMFa (Kurjan and Herskowitz, (1982) Cell:933-943), pJRY88 (Schultz et al., (1987) Gene 54:113-123), and pYES2 (Invitrogen Corporation, San Diego, Calif.).

Alternatively, an hCAR gene can be expressed in insect cells using, for example, baculovirus expression vectors. Baculovirus vectors available for expression of proteins in cultured insect cells (e.g., Sf 9 cells) include the pAc series (Smith et al. (1983) Mol. Cell. Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements.

For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed, Cold Spring Harbor Laboratory, Cold Spring Harbor Laboratory Press, Cold Spring Harbor, N.Y., 1989 incorporated herein by reference.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule encoding an hCAR protein cloned into the expression vector in an antisense orientation. That is, the DNA molecule is operatively linked to a regulatory sequence in a manner which allows for expression (by transcription of the DNA molecule) of an RNA molecule which is antisense to hCAR mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, hCAR protein can be expressed in bacterial cells such as E coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed, Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding the hCAR protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) hCAR protein. Accordingly, the invention further provides methods for producing hCAR protein using the host cells of the invention. In one embodiment, the method comprises culturing the host cell of invention (into which a recombinant expression vector encoding an hCAR protein has been introduced) in a suitable medium until the hCAR protein is produced. In another embodiment, the method further comprises isolating the hCAR protein from the medium or the host cell.

The host cells of the invention can also be used to produce non-human transgenic animals. The non-human transgenic animals can be used in screening assays designed to identify agents or compounds, e.g., drugs, pharmaceuticals, etc., which are capable of ameliorating detrimental symptoms of selected disorders such as nervous system disorders, e.g., psychiatric disorders or disorders affecting circadian rhythms and the sleep-wake cycle. For example, in one embodiment, a host cell of the invention is a fertilized oocyte or an embryonic stem cell into which hCAR protein-coding sequences have been introduced. Such host cells can then be used to create non-human transgenic animals in which exogenous hCAR gene sequences have been introduced into their genome or homologous recombinant animals in which endogenous hCAR gene sequences have been altered. Such animals are useful for studying the function and/or activity of an hCAR protein and for identifying and/or evaluating modulators of hCAR protein activity. As used herein, a “transgenic animal” is a non-human animal, preferably a mammal, more preferably a rodent such as a rat or mouse, in which one or more of the cells of the animal include a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous hCAR gene has been altered by homologous recombination between the endogenous gene and an exogenous DNA molecule introduced into a cell of the animal, e.g., an embryonic cell of the animal, prior to development of the animal.

A transgenic animal of the invention can be created by introducing hCAR protein encoding nucleic acid into the pronuclei of a fertilized oocyte, e.g., by microinjection, retroviral infection, and allowing the oocyte to develop in a pseudopregnant female foster animal. The human hCAR cDNA sequence of SEQ ID NO: 1 in its entirety, or a segment encoding any part of the hCAR protein, can be introduced as a transgene into the genome of a non-human animal.

Moreover, a non-human homologue of the human hCAR gene, such as a mouse hCAR gene, can be isolated based on hybridization to the human hCAR cDNA (described above) and used as a transgene. Genomic sequences that include the promoter, introns, and polyadenylation signals can also be included in the transgene to increase the efficiency or specificity of expression of the transgene. A tissue-specific regulatory sequence(s) can be operably linked to the hCAR transgene to direct expression of an hCAR protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et. Similar methods are used for production of other transgenic animals. A transgenic founder animal can be identified based upon the presence of the hCAR transgene in its genome and/or expression of hCAR mRNA in tissues or cells of the animals. A transgenic founder animal can then be used to breed additional animals carrying the transgene. Moreover, transgenic animals carrying a transgene encoding an hCAR protein can further be bred to other transgenic animals carrying other transgenes.

To create a homologous recombinant animal, a vector is prepared which contains at least a fragment of an hCAR gene into which a deletion, addition or substitution has been introduced to thereby alter, e.g., functionally disrupt, the hCAR gene. The hCAR gene can be a human gene (e.g., from a human genomic clone isolated from a human genomic library screened with the cDNA of SEQ ID NO: 1), but more preferably is a non-human homologue of a human hCAR gene. For example, a mouse hCAR gene can be isolated from a mouse genomic DNA library using the hCAR cDNA of SEQ ID NO: 1 as a probe. The mouse hCAR gene then can be used to construct a homologous recombination vector suitable for altering an endogenous hCAR gene in the mouse genome. In a preferred embodiment, the vector is designed such that, upon homologous recombination, the endogenous hCAR gene is functionally disrupted (i.e., no longer encodes a functional protein; also referred to as a “knock out” vector).

Alternatively, the vector can be designed such that, upon homologous recombination, the endogenous hCAR gene is mutated or otherwise altered but still encodes functional protein (e.g., the upstream regulatory region can be altered to thereby alter the expression of the endogenous hCAR protein). In the homologous recombination vector, the altered fragment of the hCAR gene is flanked at its 5′ and 3′ ends by additional nucleic acid of the hCAR gene to allow for homologous recombination to occur between the exogenous hCAR gene carried by the vector and an endogenous hCAR gene in an embryonic stem cell. The additional flanking hCAR nucleic acid is of sufficient length for successful homologous recombination with the endogenous gene.

Typically, several kilobases of flanking DNA (both at the 5′ and 3′ ends) are included in the vector (see for example, Thomas, K. R. and Capecchi, M. R. (1987) Cell 51:503 for a description of homologous recombination vectors). The vector is introduced into an embryonic stem cell line (e.g., by electroporation) and cells in which the introduced hCAR gene has homologously recombined with the endogenous hCAR gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos. WO 90/11354; WO 91/01140; WO 92/0968; and WO 93/04169.

In another embodiment, transgenic non-human animals can be produced which contain selected systems which allow for regulated expression of the transgene. One example of such a system is the cre/loxP recombinase system of bacteriophage P L For a description of the cre/loxP recombinase system, see, e.g., Lakso et al. (1992) PJVAS 89:6232-6236. Another example of a recombinase system is the FLP recombinase system of Saccharomyces cerevisiae (O'Gon-nan et al. (1991) Science 251:1351-1355). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 3 8 5:810-813 and PCT International Publication Nos. WO 97/07668 and WO 97/07669. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to pseudopregnant female foster animal. The offspring borne of this female foster animal will be a clone of the animal from which the cell, e.g., the somatic cell, is isolated. V. Uses and Methods of the Invention The nucleic acid molecules, proteins, protein homologues, modulators, and antibodies described herein can be used in one or more of the following methods: a) drug screening assays; b) diagnostic assays particularly in disease identification, allelic screening and pharmacogenetic testing; c) methods of treatment; d) pharmacogenomics; and e) monitoring of effects during clinical trials. An hCAR protein of the invention can be used as a drug target for developing agents to modulate the activity of the hCAR protein (a GPCR). The isolated nucleic acid molecules of the invention can be used to express hCAR protein (e.g., via a recombinant expression vector in a host cell or in gene therapy applications), to detect hCAR mRNA (e.g., in a biological sample) or a naturally occurring or recombinantly generated genetic mutation in a hCAR gene, and to modulate hCAR protein activity, as described further below. In addition, the hCAR proteins can be used to screen drugs or compounds which modulate hCAR protein activity. Moreover, the anti-hCAR antibodies of the invention can be used to detect and isolate an hCAR protein, particularly fragments of an hCAR protein present in a biological sample, and to modulate hCAR protein activity.

hCAR Gene Activation

The present invention also relates to improved methods for both the in vitro production of hCAR proteins and for the production and delivery of hCAR proteins by gene therapy. The present invention includes approaches which activate expression of endogenous cellular genes, and further allows amplification of the activated endogenous cellular genes, which does not require in vitro manipulation and transfection of exogenous DNA encoding hCAR proteins. These methods are described in PCT Application WO 94/12650, U.S. Pat. No. 5,968,502, and Harrington et al., Nature Biotechnology (2001) 19:440-445, all of which are incorporated in their entirety by reference. These, and variations of them which one skilled in the art will recognize as equivalent, may collectively be referred to as “gene activation”.

The present invention relates to transfected cells, both transfected primary or secondary cells (i.e., non-immortalized cells) and transfected immortalized cells, useful for producing proteins, methods of making such cells, methods of using the cells for in vitro protein production and methods of gene therapy. Cells of the present invention are of vertebrate origin, particularly of mammalian origin and even more particularly of human origin. Cells produced by the method of the present invention contain exogenous DNA which encodes a therapeutic product, exogenous DNA which is itself a therapeutic product and/or exogenous DNA which causes the transfected cells to express a gene at a higher level or with a pattern of regulation or induction that is different than occurs in the corresponding nontransfected cell.

The present invention also relates to methods by which primary, secondary, and immortalized cells are transfected to include exogenous genetic material, methods of producing clonal cell strains or heterogenous cell strains, and methods of immunizing animals, or producing antibodies in immunized animals, using the transfected primary, secondary, or immortalized cells.

The present invention relates particularly to a method of gene targeting or homologous recombination in cells of vertebrate, particularly mammalian, origin. That is, it relates to a method of introducing DNA into primary, secondary, or immortalized cells of vertebrate origin through homologous recombination, such that the DNA is introduced into genomic DNA of the primary, secondary, or immortalized cells at a preselected site. The targeting sequences used are determined by (selected with reference to) the site into which the exogenous DNA is to be inserted. The genomic hCAR sequences provided by the present invention (SEQ ID NO: 3) are useful in these methods. The present invention further relates to homologously recombinant primary, secondary, or immortalized cells, referred to as homologously recombinant (HR) primary, secondary or immortalized cells, produced by the present method and to uses of the HR primary, secondary, or immortalized cells.

The present invention also relates to a method of activating (i.e., turning on) a hCAR gene present in primary, secondary, or immortalized cells of vertebrate origin, which is normally not expressed in the cells or is not expressed at physiologically significant levels in the cells as obtained. According to the present method, homologous recombination is used to replace or disable the regulatory region normally associated with the gene in cells as obtained with a regulatory sequence which causes the gene to be expressed at levels higher than evident in the corresponding nontransfected cell, or to display a pattern of regulation or induction that is different than evident in the corresponding nontransfected cell. The present invention, therefore, relates to a method of making proteins by turning on or activating an endogenous gene which encodes the desired product in transfected primary, secondary, or immortalized cells.

In one embodiment, the activated gene can be further amplified by the inclusion of a selectable marker gene which has the property that cells containing amplified copies of the selectable marker gene can be selected for by culturing the cells in the presence of the appropriate selectable agent. The activated endogenous gene which is near or linked to the amplified selectable marker gene will also be amplified in cells containing the amplified selectable marker gene. Cells containing many copies of the activated endogenous gene are useful for in vitro protein production and gene therapy.

Transfected cells of the present invention are useful in a number of applications in humans and animals. In one embodiment, the cells can be implanted into a human or an animal for hCAR protein delivery in the human or animal. hCAR protein can be delivered systemically or locally in humans for therapeutic benefits. Barrier devices, which contain transfected cells which express a therapeutic hCAR protein product and through which the therapeutic product is freely permeable, can be used to retain cells in a fixed position in vivo or to protect and isolate the cells from the host's immune system. Barrier devices are particularly useful and allow transfected immortalized cells, transfected cells from another species (transfected xenogeneic cells), or cells from a nonhistocompatibility-matched donor (transfected allogeneic cells) to be implanted for treatment of human or animal conditions. Barrier devices also allow convenient short-term (i.e., transient) therapy by providing ready access to the cells for removal when the treatment regimen is to be halted for any reason. Transfected xenogeneic and allogeneic cells may be used for short-term gene therapy, such that the gene product produced by the cells will be delivered in vivo until the cells are rejected by the host's immune system.

Transfected cells of the present invention are also useful for eliciting antibody production or for immunizing humans and animals against pathogenic agents. Implanted transfected cells can be used to deliver immunizing antigens that result in stimulation of the host's cellular and humoral immune responses. These immune responses can be designed for protection of the host from future infectious agents (i.e., for vaccination), to stimulate and augment the disease-fighting capabilities directed against an ongoing infection, or to produce antibodies directed against the antigen produced in vivo by the transfected cells that can be useful for therapeutic or diagnostic purposes. Removable barrier devices can be used to allow a simple means of terminating exposure to the antigen. Alternatively, the use of cells that will ultimately be rejected (xenogeneic or allogeneic transfected cells) can be used to limit exposure to the antigen, since antigen production will cease when the cells have been rejected.

The methods of the present invention can be used to produce primary, secondary, or immortalized cells producing hCAR protein products or anti-sense RNA. Additionally, the methods of the present invention can be used to produce cells which produce non-naturally occurring ribozymes, proteins, or nucleic acids which are useful for in vitro production of a hCAR therapeutic product or for gene therapy.

### Drug Screening Assays

The invention provides methods for identifying compounds or agents that can be used to treat disorders characterized by (or associated with) aberrant or abnormal hCAR nucleic acid expression and/or hCAR protein activity. These methods are also referred to herein as drug screening assays and typically include the step of screening a candidate/test compound or agent to identify compounds that are an agonist or antagonist of an hCAR protein, and specifically for the ability to interact with (e.g., bind to) an hCAR protein, to modulate the interaction of an hCAR protein and a target molecule, and/or to modulate hCAR nucleic acid expression and/or hCAR protein activity. Candidate/test compounds or agents which have one or more of these abilities can be used as drugs to treat disorders characterized by aberrant or abnormal hCAR nucleic acid expression and/or hCAR protein activity. Example candidate/test compounds include: 1) peptides such as soluble peptides, including Ig-tailed fusion peptides and members of random peptide libraries and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab′)2, Fab expression library fragments, and epitope-binding fragments of antibodies); and 4) small organic and inorganic molecules (e.g., molecules obtained from combinatorial and natural product libraries). In one embodiment, the invention provides assays for screening candidate/test compounds which interact with (e.g., bind to) an hCAR protein. Typically, the assays are recombinant cell based or cell-free assays which include the steps of combining a cell expressing an hCAR protein or a bioactive fragment thereof, a membrane preparation from an hCAR expressing cells, or an isolated hCAR protein, and a candidate/test compound, e.g., under conditions which allow for interaction of (e.g., binding of) the candidate/test compound to the hCAR protein or fragment thereof to form a complex, and detecting the formation of a complex, in which the ability of the candidate compound to interact with (e.g., bind to) the hCAR protein or fragment thereof is indicated by the presence of the candidate compound in the complex. Formation of complexes between the hCAR protein and the candidate compound can be detected using competition binding assays, and can be quantitated, for example, using standard immunoassays.

In another embodiment, the invention provides screening assays to identify candidate/test compounds which modulate (e.g., stimulate or inhibit) the interaction (and most likely hCAR protein activity as well) between an hCAR protein and a molecule (target molecule) with which the hCAR protein normally interacts. Examples of such target molecules include proteins in the same signaling path as the hCAR protein, e.g., proteins which may function upstream (including both stimulators and inhibitors of activity) or downstream of the hCAR protein in, for example, a cognitive function signaling pathway or in a pathway involving hCAR protein activity, e.g., a G protein or other interactor involved in cAMP or phosphatidylinositol turnover, and/or adenylate cyclase or phospholipase C activation. Typically, the assays are recombinant cell based assays which include the steps of combining a cell expressing an hCAR protein, or a bioactive fragment thereof, an hCAR protein target molecule (e.g., an hCAR ligand) and a candidate/test compound, e.g., under conditions wherein but for the presence of the candidate compound, the hCAR protein or biologically active fragment thereof interacts with (e.g., binds to) the target molecule, and detecting the formation of a complex which includes the hCAR protein and the target molecule or detecting the interaction/reaction of the hCAR protein and the target molecule. Detection of complex formation can include direct quantitation of the complex by, for example, measuring inductive effects of the hCAR protein. A statistically significant change, such as a decrease, in the interaction of the hCAR protein and target molecule (e.g., in the formation of a complex between the hCAR protein and the target molecule) in the presence of a candidate compound (relative to what is detected in the absence of the candidate compound) is indicative of a modulation (e.g., stimulation or inhibition) of the interaction between the hCAR protein and the target molecule. Modulation of the formation of complexes between the hCAR protein and the target molecule can be quantitated using, for example, an immunoassay.

To perform cell free drug screening assays, it is desirable to immobilize either the hCAR protein or its target molecule to facilitate separation of complexes from uncomplexed forms of one or both of the proteins, as well as to accommodate automation of the assay. Interaction (e.g., binding of) of the hCAR protein to a target molecule, in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows the protein to be bound to a matrix. For example, glutathione-S-transferase/hCAR fusion proteins can be adsorbed onto glutathione sepharose beads (Sigma Chemical, St. Louis, Mo.) or glutathione derivatized microtitre plates, which are then combined with the cell lysates (e.g., 35S_labeled) and the candidate compound, and the mixture incubated under conditions conducive to complex formation (e.g., at physiological conditions for salt and pH). Following incubation, the beads are washed to remove any unbound label, and the matrix immobilized and radiolabel determined directly, or in the supernatant after the complexes are dissociated. Alternatively, the complexes can be dissociated from the matrix, separated by SDS-PAGE, and the level of hCAR-binding protein found in the bead fraction quantitated from the gel using standard electrophoretic techniques.

Other techniques for immobilizing proteins on matrices can also be used in the drug screening assays of the invention. For example, either the hCAR protein or its target molecule can be immobilized utilizing conjugation of biotin and streptavidin.

Biotinylated hCAR protein molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with an hCAR protein but which do not interfere with binding of the protein to its target molecule can be derivatized to the wells of the plate, and hCAR protein trapped in the wells by antibody conjugation. As described above, preparations of an hCAR-binding protein and a candidate compound are incubated in the hCAR protein-presenting wells of the plate, and the amount of complex trapped in the well can be quantitated. Methods for detecting such complexes, in addition to those described above for the GST-immobilized complexes, include immunodetection of complexes using antibodies reactive with the hCAR protein target molecule, or which are reactive with hCAR protein and compete with the target molecule; as well as enzyme-linked assays which rely on detecting an enzymatic activity associated with the target molecule.

In yet another embodiment, the invention provides a method for identifying a compound (e.g., a screening assay) capable of use in the treatment of a disorder characterized by (or associated with) aberrant or abnormal hCAR nucleic acid expression or hCAR protein activity. This method typically includes the step of assaying the ability of the compound or agent to modulate the expression of the hCAR nucleic acid or the activity of the hCAR protein thereby identifying a compound for treating a disorder characterized by aberrant or abnormal hCAR nucleic acid expression or hCAR protein activity. Methods for assaying the ability of the compound or agent to modulate the expression of the hCAR nucleic acid or activity of the hCAR protein are typically cell-based assays. For example, cells which are sensitive to ligands which transduce signals via a pathway involving an hCAR protein can be induced to overexpress an hCAR protein in the presence and absence of a candidate compound.

Candidate compounds which produce a statistically significant change in hCAR protein-dependent responses (either stimulation or inhibition) can be identified. In one embodiment, expression of the hCAR nucleic acid or activity of an hCAR protein is modulated in cells and the effects of candidate compounds on the readout of interest (such as cAMP or phosphatidylinositol turnover) are measured. For example, the expression of genes which are up- or down-regulated in response to an hCAR protein-dependent signal cascade can be assayed. In preferred embodiments, the regulatory regions of such genes, e.g., the 5′ flanking promoter and enhancer regions, are operably linked to a detectable marker (such as luciferase) which encodes a gene product that can be readily detected. Phosphorylation of an hCAR protein or hCAR protein target molecules can also be measured, for example, by immunoblotting.

Alternatively, modulators of hCAR gene expression (e.g., compounds which can be used to treat a disorder characterized by aberrant or abnormal hCAR nucleic acid expression or hCAR protein activity) can be identified in a method wherein a cell is contacted with a candidate compound and the expression of hCAR mRNA or protein in the cell is determined. The level of expression of hCAR mRNA or protein in the presence of the candidate compound is compared to the level of expression of hCAR mRNA or protein in the absence of the candidate compound. The candidate compound can then be identified as a modulator of hCAR nucleic acid expression based on this comparison and be used to treat a disorder characterized by aberrant hCAR nucleic acid expression. For example, when expression of hCAR mRNA or protein is greater (statistically significantly greater) in the presence of the candidate compound than in its absence, the candidate compound is identified as a stimulator of hCAR nucleic acid expression. Alternatively, when hCAR nucleic acid expression is less (statistically significantly less) in the presence of the candidate compound than in its absence, the candidate compound is identified as an inhibitor of hCAR nucleic acid expression. The level of hCAR nucleic acid expression in the cells can be determined by methods described herein for detecting hCAR mRNA or protein.

Additional, typical screening assays include those described in U.S. Pat. Nos. 5,691,188; 5,846,819; and international application publication number WO 01/09184 at page 26, all of which assays are incorporated by reference.

In yet another aspect of the invention, the hCAR proteins, or fragments thereof, can be used as “bait proteins” in a two-hybrid assay (see, e.g., U.S. Pat. No. 5,283,317; Zervos et al. (1993) Cell 72:223-232; Madura et al. (1993) J Biol. Chem. 268:12046-12054; Bartel et al. (1993) Biotechniques 14:920-924; Iwabuchi et al. (1993) Oncogene 8:1693-1696; and Brent WO 94/10300), to identify other proteins, which bind to or interact with the hCAR protein (“hCAR-binding proteins” or “hCAR-bp”) and modulate hCAR protein activity. Such hCAR-binding proteins are also likely to be involved in the propagation of signals by the hCAR proteins as, for example, upstream or downstream elements of the hCAR protein pathway.

The two-hybrid system is based on the modular nature of most transcription factors, which consist of separable DNA-binding and activation domains. Bartel, P. et al. “Using the Two-Hybrid System to Detect Protein-Protein Interactions” in Cellular Interactions in Development: A Practical Approach, Hartley, D. A. ed. (Oxford University Press, Oxford, 1993) pp. 153-179. Briefly, the assay utilizes two different DNA constructs. In one construct, the gene that encode an hCAR protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an hCAR-protein dependent complex, the DNA-binding and activation domains of the transcription factor are brought into close proximity. This proximity allows transcription of a reporter gene (e.g., LacZ) which is operably linked to a transcriptional regulatory site responsive to the transcription factor.

Expression of the reporter gene can be detected and cell colonies containing the functional transcription factor can be isolated and used to obtain the cloned gene which encodes the protein which interacts with the hCAR protein.

Modulators of hCAR protein activity and/or hCAR nucleic acid expression identified according to these drug screening assays can be used to treat, for example, nervous system disorders. These methods of treatment include the steps of administering the modulators of hCAR protein activity and/or nucleic acid expression, e.g., in a pharmaceutical composition as described herein, to a subject in need of such treatment, e.g., a subject with a disorder described herein.

### Diagnostic Assays

The invention further provides a method for detecting the presence of an hCAR protein or hCAR nucleic acid molecule, or fragment thereof, in a biological sample.

The method involves contacting the biological sample with a compound or an agent capable of detecting hCAR protein or mRNA such that the presence of hCAR protein/encoding nucleic acid molecule is detected in the biological sample. A preferred agent for detecting hCAR mRNA is a labeled or labelable nucleic acid probe capable of hybridizing to hCAR mRNA. The nucleic acid probe can be, for example, the full-length hCAR cDNA of SEQ ID NO: 1, or a fragment thereof, such as an oligonucleotide of at least 15, 30, 50, 100, 250 or 500 nucleotides in length and sufficient to specifically hybridize under stringent conditions to hCAR mRNA. A preferred agent for detecting hCAR protein is a labeled or labelable antibody capable of binding to hCAR protein. Antibodies can be polyclonal, or more preferably, monoclonal. An intact antibody, or a fragment thereof (e.g., Fab or F(ab′)2) can be used. The term “labeled or labelable,” with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect hCAR mRNA or protein in a biological sample in vitro as well as in vivo. For example, in vitro techniques for detection of hCAR mRNA include Northern hybridizations and in situ hybridizations. In vitro techniques for detection of hCAR protein include enzyme linked immunosorbent assays (ELISAs), Western blots, immunoprecipitations and immunofluorescence. Alternatively, hCAR protein can be detected in vivo in a subject by introducing into the subject a labeled anti-hCAR antibody. For example, the antibody can be labeled with a radioactive marker whose presence and location in a subject can be detected by standard imaging techniques. Particularly useful are methods which detect the allelic variant of an hCAR protein expressed in a subject and methods which detect fragments of an hCAR protein in a sample.

The invention also encompasses kits for detecting the presence of an hCAR protein in a biological sample. For example, the kit can comprise reagents such as a labeled or labelable compound or agent capable of detecting hCAR protein or mRNA in a biological sample; means for determining the amount of hCAR protein in the sample; and means for comparing the amount of hCAR protein in the sample with a standard. The compound or agent can be packaged in a suitable container. The kit can further comprise instructions for using the kit to detect hCAR mRNA or protein.

The methods of the invention can also be used to detect naturally occurring genetic mutations in a hCAR gene, thereby determining if a subject with the mutated gene is at risk for a disorder characterized by aberrant or abnormal hCAR nucleic acid expression or hCAR protein activity as described herein. In preferred embodiments, the methods include detecting, in a sample of cells from the subject, the presence or absence of a genetic mutation characterized by at least one of an alteration affecting the integrity of a gene encoding an hCAR protein, or the misexpression of the hCAR gene. For example, such genetic mutations can be detected by ascertaining the existence of at least one of 1) a deletion of one or more nucleotides from a hCAR gene; 2) an addition of one or more nucleotides to a hCAR gene; 3) a substitution of one or more nucleotides of a hCAR gene, 4) a chromosomal rearrangement of a hCAR gene; 5) an alteration in the level of a messenger RNA transcript of an hCAR gene, 6) aberrant modification of a hCAR gene, such as of the methylation pattern of the genomic DNA, 7) the presence of a non-wild type splicing pattern of a messenger RNA transcript of a hCAR gene, 8) a non-wild type level of an hCAR-protein, 9) allelic loss of an hCAR gene, and 10) inappropriate post-translational modification of an hCAR-protein. As described herein, there are a large number of assay techniques known in the art that can be used for detecting mutations in a hCAR gene.

In certain embodiments, detection of the mutation involves the use of a probe/primer in a polymerase chain reaction (PCR) (see, e.g. U.S. Pat. Nos. 4,683,195 and 4,683,202), such as anchor PCR or RACE PCR, or, alternatively, in a ligation chain reaction (LCR), the latter of which can be particularly useful for detecting point mutations in the hCAR-gene (see Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to a hCAR gene under conditions such that hybridization and amplification of the hCAR-gene (if present) occurs, and detecting the presence or absence of an amplification product, or detecting the size of the amplification product and comparing the length to a control sample.

In an alternative embodiment, mutations in a hCAR gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see U.S. Pat. No. 5,498,531 hereby incorporated by reference in its entirety) can be used to score for the presence of specific mutations by development or loss of a ribozyme cleavage site.

In yet another embodiment, any of a variety of sequencing reactions known in the art can be used to directly sequence the hCAR gene and detect mutations by comparing the sequence of the sample hCAR gene with the corresponding wild-type (control) sequence. Examples of sequencing reactions include those based on techniques developed by Maxim and Gilbert ((1977) PNAS 74:560) or Sanger ((1977) PNAS 74:5463). A variety of automated sequencing procedures can be utilized when performing the diagnostic assays ((1995) Biotechniques 19:448), including sequencing by mass spectrometry (see, e.g., PCT International Publication No. WO 94/1610 1; Cohen et al. (1996) Adv. Chromatogr. 36:127-162; and Griffin et al. (1993) Appl. Biochem. Biotechnol. 38:147-159).

Other methods for detecting mutations in the hCAR gene include methods in which protection from cleavage agents is used to detect mismatched bases in RNA/RNA or RNA/DNA duplexes (Myers et al. (1985) Science 230:1242); Cotton et al. (1988) PNAS 85:4397; Saleeba et al. (1992) Meth. Enzymol. 217:286-295), electrophoretic mobility of mutant and wild type nucleic acid is compared (Orita et al. (1989) PNAS 86:2766; Cotton (1993) Mutat. Res. 285:125-144; and Hayashi (1992) Genet. Anal. Tech. Appl. 9:73-79), and movement of mutant or wild-type fragments in polyacrylamide gels containing a gradient of denaturant is assayed using denaturing gradient gel electrophoresis (Myers et al (1985) Nature 313:495). Examples of other techniques for detecting point mutations include, selective oligonucleotide hybridization, selective amplification, and selective primer extension.

### Methods of Treatment

Another aspect of the invention pertains to methods for treating a subject, e.g., a human, having a disease or disorder characterized by (or associated with) aberrant or abnormal hCAR nucleic acid expression and/or hCAR protein activity. These methods include the step of administering an hCAR protein/gene modulator (agonist or antagonist) to the subject such that treatment occurs. The language “aberrant or abnormal hCAR protein expression” refers to expression of a non-wild-type hCAR protein or a non-wild-type level of expression of an hCAR protein. Aberrant or abnormal hCAR protein activity refers to a non-wild-type hCAR protein activity or a non-wild-type level of hCAR protein activity. As the hCAR protein is involved in a pathway involving signaling within cells, aberrant or abnormal hCAR protein activity or expression interferes with the normal regulation of functions mediated by hCAR protein signaling, and in particular brain cells.

The terms “treating” or “treatment,” as used herein, refer to reduction or alleviation of at least one adverse effect or symptom of a disorder or disease, e.g., a disorder or disease characterized by or associated with abnormal or aberrant hCAR protein activity or hCAR nucleic acid expression.

As used herein, an hCAR protein/gene modulator is a molecule which can modulate hCAR nucleic acid expression and/or hCAR protein activity. For example, an hCAR gene or protein modulator can modulate, e.g., upregulate (activate/agonize) or downregulate (suppress/antagonize), hCAR nucleic acid expression. In another example, an hCAR protein/gene modulator can modulate (e.g., stimulate/agonize or inhibit/antagonize) hCAR protein activity. If it is desirable to treat a disorder or disease characterized by (or associated with) aberrant or abnormal (non-wild-type) hCAR nucleic acid expression and/or hCAR protein activity by inhibiting hCAR nucleic acid expression, an hCAR modulator can be an antisense molecule, e.g., a ribozyme, as described herein. Examples of antisense molecules which can be used to inhibit hCAR nucleic acid expression include antisense molecules which are complementary to a fragment of the 5′ untranslated region of SEQ ID NO: 1 which also includes the start codon and antisense molecules which are complementary to a fragment of the 3′ untranslated region of SEQ ID NO: 1.

An hCAR modulator that inhibits hCAR nucleic acid expression can also be a small molecule or other drug, e.g., a small molecule or drug identified using the screening assays described herein, which inhibits hCAR nucleic acid expression. If it is desirable to treat a disease or disorder characterized by (or associated with) aberrant or abnormal (non-wild-type) hCAR nucleic acid expression and/or hCAR protein activity by stimulating hCAR nucleic acid expression, an hCAR modulator can be, for example, a nucleic acid molecule encoding an hCAR protein (e.g., a nucleic acid molecule comprising a nucleotide sequence homologous to the nucleotide sequence of SEQ ID NO: 1) or a small molecule or other drug, e.g., a small molecule (peptide) or drug identified using the screening assays described herein, which stimulates hCAR nucleic acid expression.

Alternatively, if it is desirable to treat a disease or disorder characterized by (or associated with) aberrant or abnormal (non-wild-type) hCAR nucleic acid expression and/or hCAR protein activity by inhibiting hCAR protein activity, an hCAR modulator can be an anti-hCAR antibody or a small molecule or other drug, e.g., a small molecule or drug identified using the screening assays described herein, which inhibits hCAR protein activity. The extracellular regions of hCAR identified in the present application represent particularly good antigenic targets for therapeutic intervention. Therefore antibodies raised against peptides comprising any sequence as disclosed in SEQ ID NOs: 4, 5, 6, or 7 are useful in the present invention. If it is desirable to treat a disease or disorder characterized by (or associated with) aberrant or abnormal (non-wild-type) hCAR nucleic acid expression and/or hCAR protein activity by stimulating hCAR protein activity, an hCAR modulator can be an active hCAR protein or fragment thereof (e.g., an hCAR protein or fragment thereof having an amino acid sequence which is homologous to the amino acid sequence of SEQ ID NO: 2 or a fragment thereof) or a small molecule or other drug, e.g., a small molecule or drug identified using the screening assays described herein, which stimulates hCAR protein activity.

Other aspects of the invention pertain to methods for modulating an hCAR protein mediated cell activity. These methods include contacting the cell with an agent (or a composition which includes an effective amount of an agent) which modulates hCAR protein activity or hCAR nucleic acid expression such that an hCAR protein mediated cell activity is altered relative to normal levels (for example, cAMP or phosphatidylinositol metabolism). As used herein, “an hCAR protein mediated cell activity” refers to a normal or abnormal activity or function of a cell. Examples of hCAR protein mediated cell activities include phosphatidylinositol turnover, calcium concentrations, reporter transgenes, production or secretion of molecules, such as proteins, contraction, proliferation, migration, differentiation, and cell survival. In a preferred embodiment, the cell is neural cell of the brain, e.g., a hippocampal cell. The term “altered” as used herein refers to a change, e.g., an increase or decrease, of a cell associated activity particularly cAMP or phosphatidylinositol turnover, and adenylate cyclase or phospholipase C activation.

In one embodiment, the agent stimulates hCAR protein activity or hCAR nucleic acid expression. In another embodiment, the agent inhibits hCAR protein activity or hCAR nucleic acid expression. These modulatory methods can be performed in vitro (e.g., by culturing the cell with the agent) or, alternatively, in vivo (e.g., by administering the agent to a subject). In a preferred embodiment, the modulatory methods are performed in vivo, i.e., the cell is present within a subject, e.g., a mammal, e.g., a human, and the subject has a disorder or disease characterized by or associated with abnormal or aberrant hCAR protein activity or hCAR nucleic acid expression.

A nucleic acid molecule, a protein, an hCAR modulator, a compound etc. used in the methods of treatment can be incorporated into an appropriate pharmaceutical composition described below and administered to the subject through a route which allows the molecule, protein, modulator, or compound etc. to perform its intended function.

Disorders involving the brain include, but are not limited to, disorders involving neurons, and disorders involving glia, such as astrocytes, oligodendrocytes, ependymal cells, and microglia; cerebral edema, raised intracranial pressure and herniation, and hydrocephalus; malformations and developmental diseases, such as neural tube defects, forebrain anomalies, posterior fossa anomalies, and syringomyelia and hydromyelia; perinatal brain injury; cerebrovascular diseases, such as those related to hypoxia, ischemia, and infarction, including hypotension, hypoperfusion, and low-flow states—global cerebral ischemia and focal cerebral ischemia—infarction from obstruction of local blood supply, intracranial hemorrhage, including intracerebral (intraparenchymal) hemorrhage, subarachnoid hemorrhage and ruptured berry aneurysms, and vascular malformations, hypertensive cerebrovascular disease, including lacunar infarcts, slit hemorrhages, and hypertensive encephalopathy; infections, such as acute meningitis, including acute pyogenic (bacterial) meningitis and acute aseptic (viral) meningitis, acute focal suppurative infections, including brain abscess, subdural empyema, and extradural abscess, chronic bacterial meningoencephalitis, including tuberculosis and mycobacterioses, neurosyphilis, and neuroborreliosis (Lyme disease), viral meningoencephalitis, including arthropod-borne (Arbo) viral encephalitis, Herpes simplex virus Type 1, Herpes simplex virus Type 2, Varicalla-zoster virus (Herpes zoster), cytornegalovirus, poliomyelitis, rabies, and human immunodeficiency virus 1, including FHV-I meningoencephalitis (subacute encephalitis), vacuolar myelopathy, AIDS-associated myopathy, peripheral neuropathy, and AIDS in children, progressive multifocal leukoencephalopathy, subacute sclerosing panencephalitis, fungal meningoencephalitis, other infectious diseases of the nervous system; transmissible spongiform encephalopathies (prion diseases); demyelinating diseases, including multiple sclerosis, multiple sclerosis variants, acute disseminated encephalomyelitis and acute necrotizing hemorrhagic encephalomyelitis, and other diseases with demyelination; degenerative diseases, such as degenerative diseases affecting the cerebral cortex, including Alzheimer disease and Pick disease, degenerative diseases of basal ganglia and brain stem, including Parkinsonism, idiopathic Parkinson disease (paralysis agitans), progressive supranuclear palsy, corticobasal degeneration, multiple system atrophy, including striatonigral degeneration, Shy-Drager syndrome, and olivopontocerebellar atrophy, and Huntington disease; spinocerebellar degenerations, including spinocerebellar ataxias, including Friedreich ataxia, and ataxia-telanglectasia, degenerative diseases affecting motor neurons, including amyotrophic lateral sclerosis (motor neuron disease), bulbospinal atrophy (Kennedy syndrome), and spinal muscular atrophy; inborn errors of metabolism, such as leukodystrophies, including Krabbe disease, metachromatic leukodystrophy, adrenoleukodystrophy, ˜elizaeus-Merzbacher disease, and Canavan disease, mitochondrial encephalomyopathies, including Leigh disease and other mitochondrial encephalomyopathies; toxic and acquired metabolic diseases, including vitamin deficiencies such as thiamine (vitamin BI) deficiency and vitamin B12 deficiency, neurologic sequelae of metabolic disturbances, including hypoglycernia, hyperglycemia, and hepatic encephatopathy, toxic disorders, including carbon monoxide, methanol, ethanol, and radiation, including combined methotrexate and radiation-induced injury; tumors, such as gliomas, including astrocytoma, including fibrillary (diffuse) astrocytoma and glioblastorna multiforme, pilocytic astrocytoma, pleomorphic xanthoastrocytorna, and brain stem glioma, oligodendrogliorna, and ependymoma and related paraventricular mass lesions, neuronal tumors, poorly differentiated neoplasms, including medulloblastoma, other parenchymal tumors, including primary brain lymphoma, germ cell tumors, and pineal parenchymal tumors, meningiomas, metastatic tumors, paraneoplastic syndromes, peripheral nerve sheath tumors, including schwarinoma, neurofibroma, and malignant peripheral nerve sheath tumor (malignant schwannoma), and neurocutaneous syndromes (phakomatoses), including neurofibromotosis, including Type I neurofibromatosis (NFI) and TYPE 2 neurofibromatosis (NF2), tuberous sclerosis, and Von Hippel-Lindau disease. Also included are neuropsychiatric disorders including but not limited to schizophrenia, episodic paraoxysmal anxiety (EPA) disorders such as obsessive compulsive disorder (OCD, post traumatic stress disorder (PTSD), phobia and panic, major depressive disorder, bipolar disorder, Parkinson's disease, general anxiety disorder, autism, delirium, multiple sclerosis, dementia and other neurodegenerative diseases, severe mental retardation, dyskinesias, Tourett's syndrome, tics, tremor, dystonia, spasms, anorexia, bulimia, stroke addiction/dependency/craving, sleep disorder epilepsy, migraine; attention deficit/hyperactivity disorder (ADHD) disorder, unipolar affective disorder, adolescent conduct disorder, and “addictions”.

### Pharmacogenomics

Test/candidate compounds, or modulators which have a stimulatory or inhibitory effect on hCAR protein activity (e.g., hCAR gene expression) as identified by a screening assay described herein can be administered to individuals to treat (prophylactically or therapeutically) disorders (e.g., neurological disorders) associated with aberrant hCAR protein activity. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permit the selection of effective compounds (e.g., drugs) for prophylactic or therapeutic treatments based on a consideration of the individual's genotype. Such pharmacogenomics can further be used to determine appropriate dosages and therapeutic regimens. Accordingly, the activity of hCAR protein, expression of hCAR nucleic acid, or mutation content of hCAR genes in an individual can be determined to thereby select appropriate compound(s) for therapeutic or prophylactic treatment of the individual.

Pharmacogenomics deal with clinically significant hereditary variations in the response to drugs due to altered drug disposition and abnormal action in affected persons. See, e.g., Eichelbaum, M. (1996) Clin. Exp. Pharmacol. Physiol. 23(10-11): 983-985 and Linder, M. W. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase deficiency (GOD) is a common inherited enzymopathy in which the main clinical complication is haemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2136 and CYP2C 19) has provided an explanation as to why some patients do not obtain the expected drug effects or show exaggerated drug response and serious toxicity after taking the standard and safe dose of a drug.

These polymorphisms are expressed in two phenotypes in the population, the extensive metabolizer (EM) and poor metabolizer (PM). The prevalence of PM is different among different populations. For example, the gene coding for CYP2136 is highly polymorphic and several mutations have been identified in PM, which all lead to the absence of functional CYP2D6. Poor metabolizers of CYP2136 and CYP2C 19 quite frequently experience exaggerated drug response and side effects when they receive standard doses.

If a metabolite is the active therapeutic moiety, PM show no therapeutic response, as demonstrated for the analgesic effect of codeine mediated by its CYP2136-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of hCAR protein, expression of hCAR nucleic acid, or mutation content of hCAR genes in an individual can be determined to thereby select appropriate agent(s) for therapeutic or prophylactic treatment of a subject. In addition, pharmacogenetic studies can be used to apply genotyping of polymorphic alleles encoding drug-metabolizing enzymes to the identification of a subject's drug responsiveness phenotype. This knowledge, when applied to dosing or drug selection, can avoid adverse reactions or therapeutic failure and thus enhance therapeutic or prophylactic efficiency when treating a subject with an hCAR modulator, such as a modulator identified by one of the exemplary screening assays described herein.

### Monitoring of Effects During Clinical Trials

Monitoring the influence of compounds (e.g., drugs) on the expression or activity of hCAR protein/gene can be applied not only in basic drug screening, but also in clinical trials. For example, the effectiveness of an agent determined by a screening assay, as described herein, to increase hCAR gene expression, protein levels, or up-regulate hCAR activity, can be monitored in clinical trials of subjects exhibiting decreased hCAR gene expression, protein levels, or down-regulated hCAR protein activity. Alternatively, the effectiveness of an agent, determined by a screening assay, to decrease hCAR gene expression, protein levels, or down-regulate hCAR protein activity, can be monitored in clinical trials of subjects exhibiting increased hCAR gene expression, protein levels, or up-regulated hCAR protein activity. In such clinical trials, the expression or activity of an hCAR protein and, preferably, other genes which have been implicated in, for example, a nervous system related disorder can be used as a “read out” or markers of the ligand responsiveness of a particular cell.

For example, and not by way of limitation, genes, including a hCAR gene, which are modulated in cells by treatment with a compound (e.g., drug or small molecule) which modulates hCAR protein/gene activity (e.g., identified in a screening assay as described herein) can be identified. Thus, to study the effect of compounds on CNS disorders, for example, in a clinical trial, cells can be isolated and RNA prepared and analyzed for the levels of expression of a hCAR gene and other genes implicated in the disorder. The levels of gene expression (i.e., a gene expression pattern) can be quantified by Northern blot analysis or RT-PCR, as described herein, or alternatively by measuring the amount of protein produced, by one of the methods described herein, or by measuring the levels of activity of an hCAR protein or other genes. In this way, the gene expression pattern can serve as an marker, indicative of the physiological response of the cells to the compound. Accordingly, this response state may be determined before, and at various points during, treatment of the individual with the compound.

In a preferred embodiment, the present invention provides a method for monitoring the effectiveness of treatment of a subject with a compound (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the compound; (ii) detecting the level of expression of an hCAR protein, mRNA, or genomic DNA in the preadministration sample; (iii) obtaining one or more post-administration samples from the subject; (iv) detecting the level of expression or activity of the hCAR protein, mRNA, or genomic DNA in the post-administration samples; (v) comparing the level of expression or activity of the hCAR protein, mRNA, or genomic DNA in the pre-administration sample with the hCAR protein, mRNA, or genomic DNA in the post administration sample or samples; and (vi) altering the administration of the compound to the subject accordingly. For example, increased administration of the compound may be desirable to increase the expression or activity of an hCAR protein/gene to higher levels than detected, i.e., to increase the effectiveness of the agent.

Alternatively, decreased administration of the agent may be desirable to decrease expression or activity of hCAR to lower levels than detected, i.e. to decrease the effectiveness of the compound.

### Pharmaceutical Compositions

The hCAR nucleic acid molecules, hCAR proteins (particularly fragments of hCAR), modulators of an hCAR protein, and anti-hCAR antibodies (also referred to herein as “active compounds”) of the invention can be incorporated into pharmaceutical compositions suitable for administration to a subject, e.g., a human. Such compositions typically comprise the nucleic acid molecule, protein, modulator, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor-ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an hCAR protein or anti-hCAR antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems.

Biodegradable, biocompatible polymers can be used, such as ethylene vinyl acetate, polyanhydrides, polyglycolic acid, collagen, polyorthoesters, and polylactic acid.

Methods for preparation of such formulations will be apparent to those skilled in the art.

The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811 which is incorporated herein by reference.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

Uses of Partial hCAR Sequences

Fragments of the cDNA sequences identified herein (and the corresponding complete gene sequences) can be used in numerous ways as polynucleotide reagents. For example, these sequences can be used to: (a) map their respective genes on a chromosome; and, thus, locate gene regions associated with genetic disease; (b) identify an individual from a minute biological sample (tissue typing); and (c) aid in forensic identification of a biological sample. These applications are described in the subsections below.

### Chromosome Mapping

Once the sequence (or a fragment of the sequence) of a gene has been isolated, this sequence can be used to map the location of the gene on a chromosome. This process is called chromosome mapping. Accordingly, fragments of a hCAR nucleic acid sequences can be used to map the location of the hCAR gene, respectively, on a chromosome. The mapping of the hCAR sequence to chromosomes is an important first step in correlating these sequence with genes associated with disease.

Briefly, the hCAR gene can be mapped to a chromosome by preparing PCR primers (preferably 15-25 bp in length) from the hCAR gene sequence. Computer analysis of the hCAR gene sequence can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the hCAR gene sequence will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme, will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. (D'Eustachio, P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the hCAR gene sequence to design oligonucleotide primers, sublocalization can be achieved with panels of fragments from specific chromosomes. Other mapping strategies which can similarly be used to map a hCAR gene sequence to its chromosome include in situ hybridization (described in Fan, Y. et al. (1990) PNAS, 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results at a reasonable amount of time.

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data (such data are found, for example, above). McKusick, Mendelian Inheritance in Man, available on-line through Johns Hopkins University Welch Medical library). The relationship between genes and disease, mapped to the same chromosomal region, can then be identified through linkage analysis (co-inheritance of physically adjacent genes).

Moreover, differences in the DNA sequences between individuals affected and unaffected with a disease associated with the hCAR gene, can be determined. If a mutation is observed in some or all of the affected individuals but not in any unaffected individuals, then the mutation is likely to be the causative agent of the particular disease.

Comparison of affected and unaffected individuals generally involves first looking for structural alterations in the chromosomes, such as deletions or translocations that are visible from chromosome spreads or detectable using PCR based on that DNA sequence.

Ultimately, complete sequencing of genes from several individuals can be performed to confirm the presence of a mutation and to distinguish mutations from polymorphisms.

Use of the sequence of hCAR in SEQ ID NO: 1 has enabled the discovery of the complete hCAR gene (SEQ ID NO: 3) and also to the chromosomal mapping of the gene to chromosome 4.

### Tissue Typing

The hCAR gene sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected fragments of an individual's genome. Thus, the hCAR sequences described herein can be used to prepare two PCR primers from the 5′ and 3′ ends of the sequences.

These primers can then be used to amplify an individual's DNA and subsequently sequence it.

Panels of corresponding DNA sequences from individuals prepared in this manner can provide unique individual identifications, as each individual will have a unique set of such DNA sequences due to allelic differences. The sequences of the present invention can be used to obtain such identification sequences from individuals and from tissue. The hCAR gene sequences of the invention uniquely represent fragments of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequence of SEQ ID NO: 1, can comfortably provide positive individual identification with a panel of perhaps 10 to 1,000 primers which each yield a noncoding amplified sequence of 100 bases. If a predicted coding sequence, such as that in SEQ ID NO: 2, is used, a more appropriate number of primers for positive individual identification would be 500-2,000. If a panel of reagents from the hCAR gene sequences described herein is used to generate a unique identification database for an individual, those same reagents can later be used to identify tissue from that individual. Using the unique identification database, positive identification of the individual, living or dead, can be made from extremely small tissue samples.

Use of Partial hCAR Gene Sequences in Forensic Biology

DNA-based identification techniques can also be used in forensic biology.

Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As described above, actual base sequence information can be used for identification as an accurate alternative to patterns formed by restriction enzyme generated fragments. Sequences targeted to the noncoding region of SEQ ID NO: 1 are particularly appropriate for this use as greater numbers of polymorphisms occur in the noncoding regions, making it easier to differentiate individuals using this technique.

Examples of polynucleotide reagents include the hCAR sequences or fragments thereof, e.g., fragments derived from the noncoding region of SEQ ID NO: 1, having a length of at least 20 bases, preferably at least 30 bases.

The hCAR sequences described herein can further be used to provide polynucleotide reagents, e.g., labeled or labelable probes which can be used in, for example, an in situ hybridization technique, to identify a specific tissue, e.g., brain or placenta tissue. This can be very useful in cases where a forensic pathologist is presented with a tissue of unknown origin. Panels of such hCAR probes can be used to identify tissue by species and/or by organ type.

In a similar fashion, these reagents, e.g., hCAR primers or probes can be used to screen tissue culture for contamination (i.e., screen for the presence of a mixture of different types of cells in a culture).

This invention is further illustrated by the following examples which should not be construed as limiting. The contents of all references, patent applications, patents, and published patent applications cited throughout this application are hereby incorporated by reference.

### EXAMPLES

### Example 1

### Identification of Human hCAR cDNA

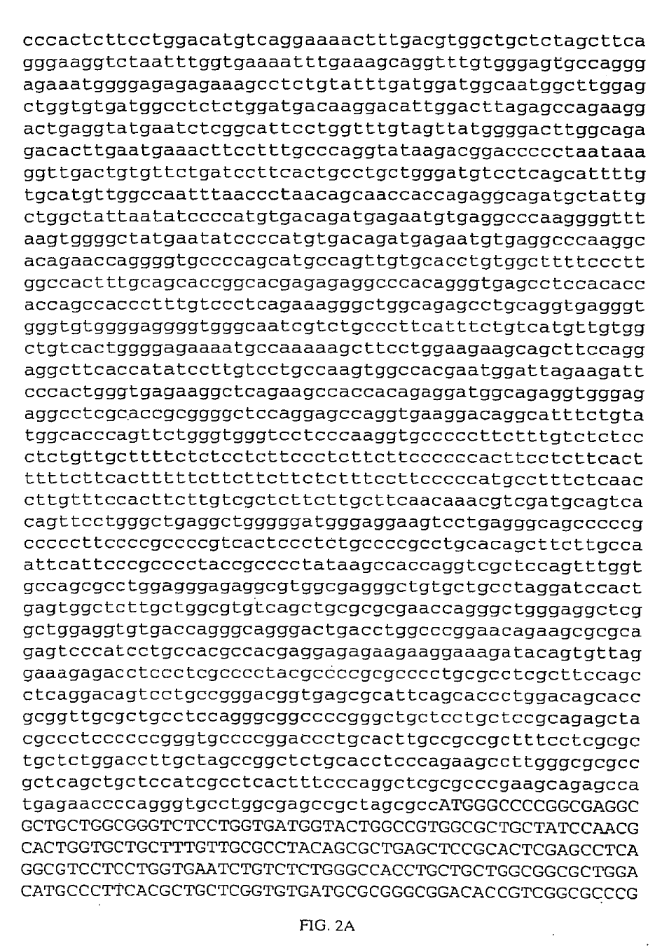

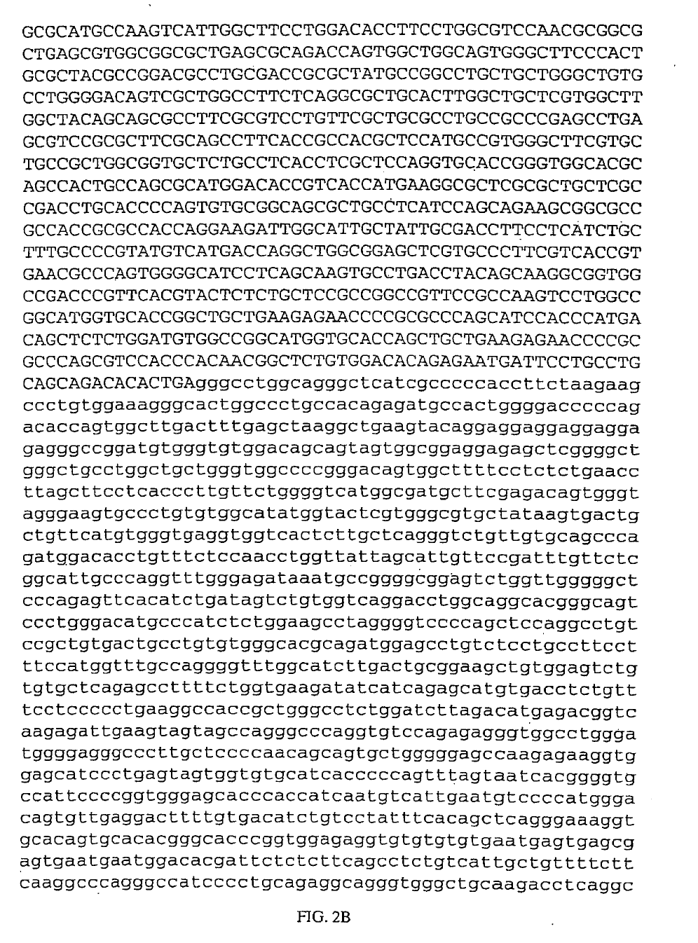

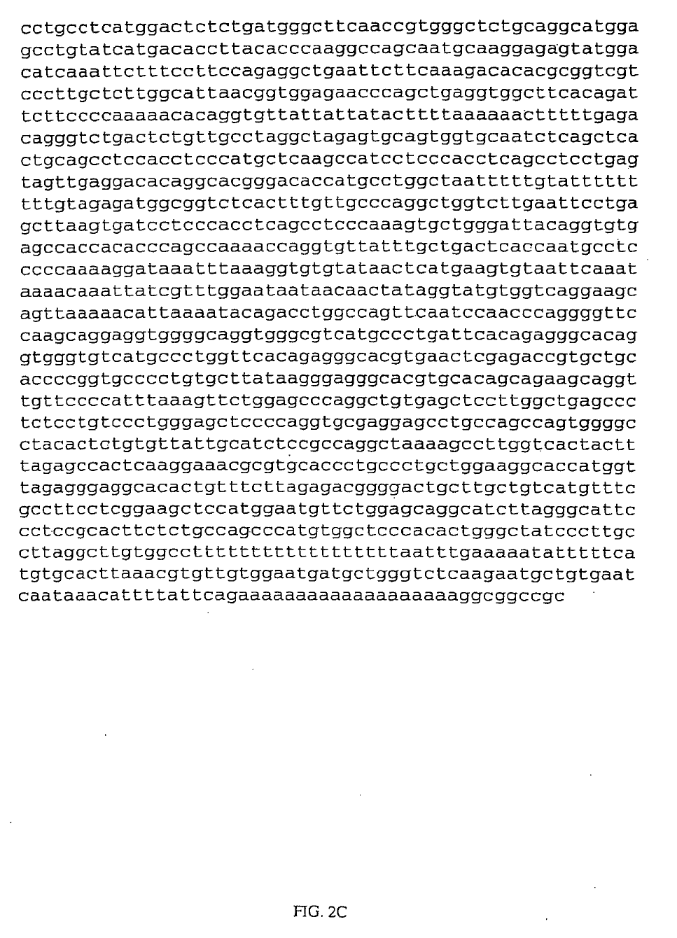

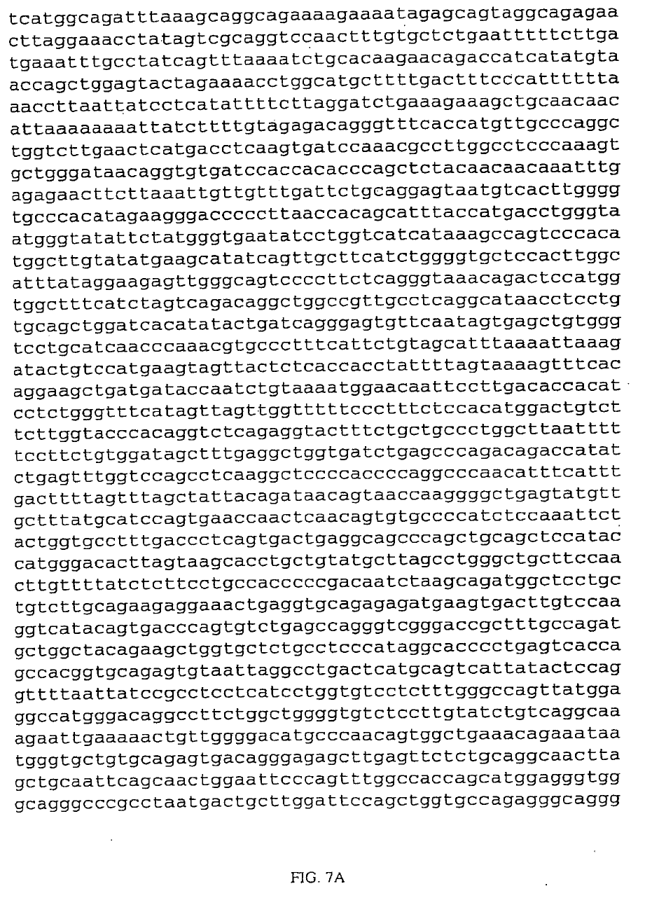

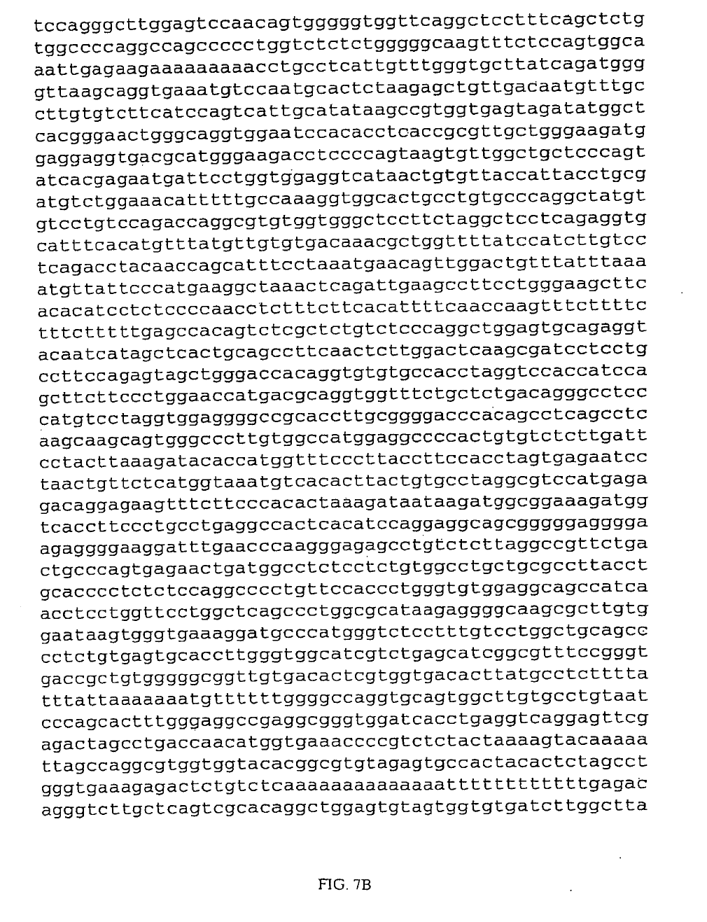

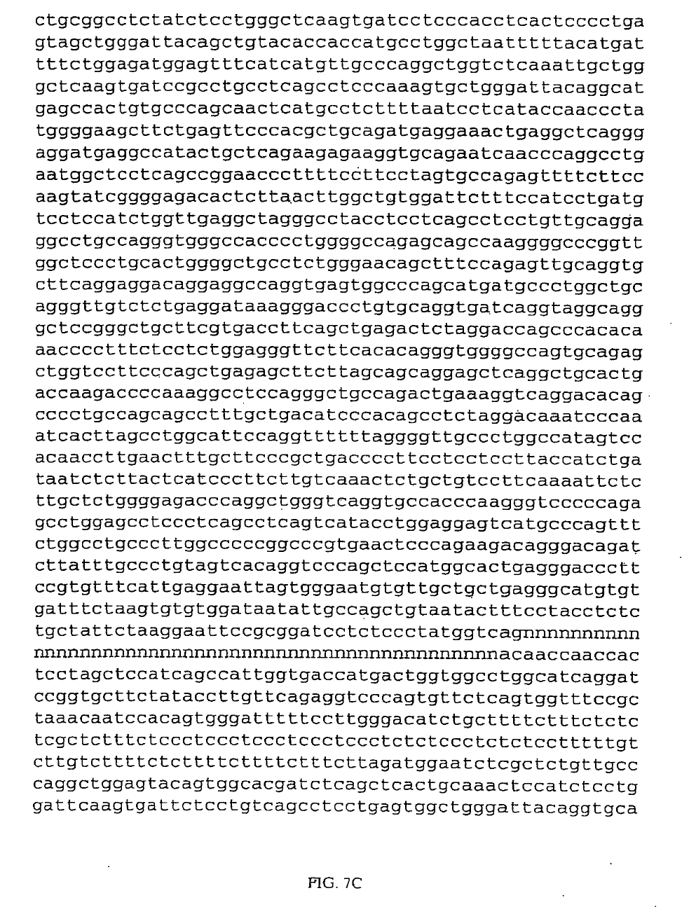

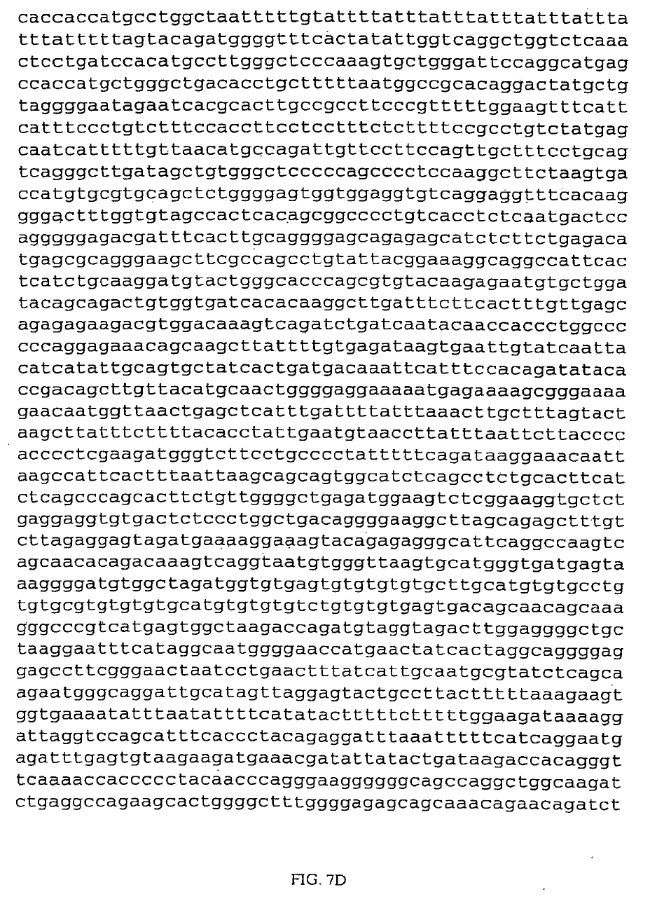

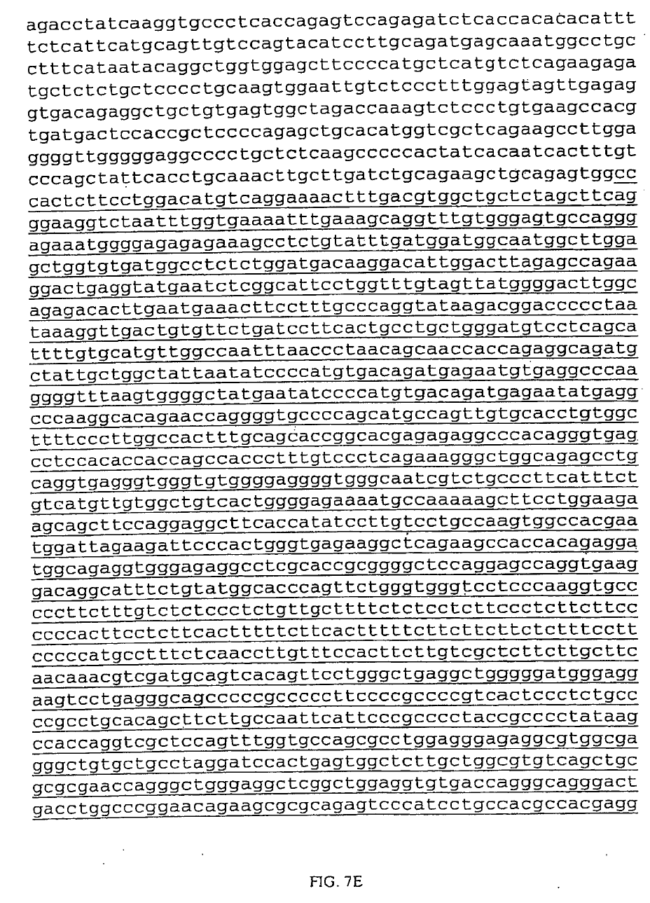

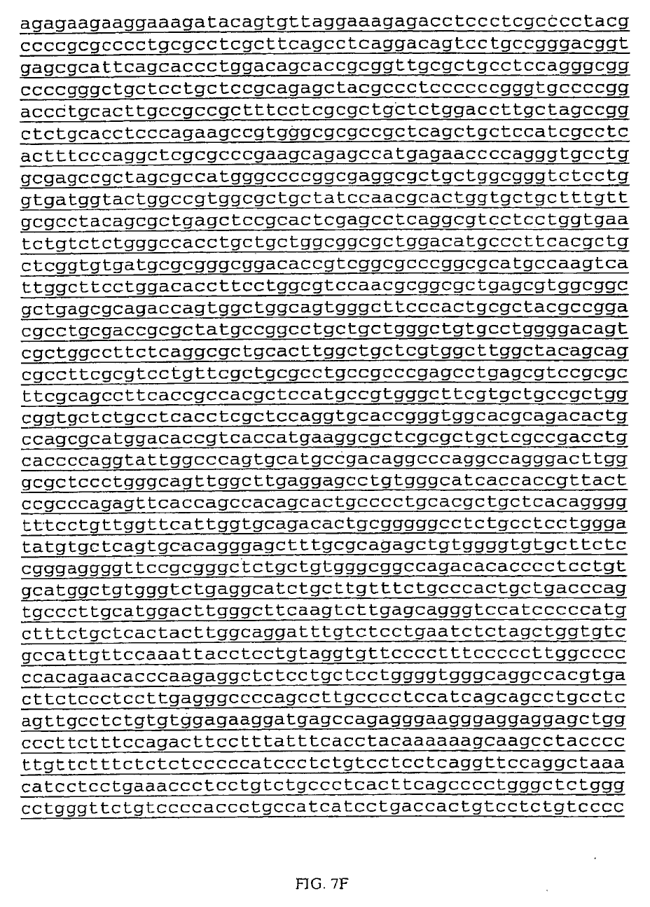

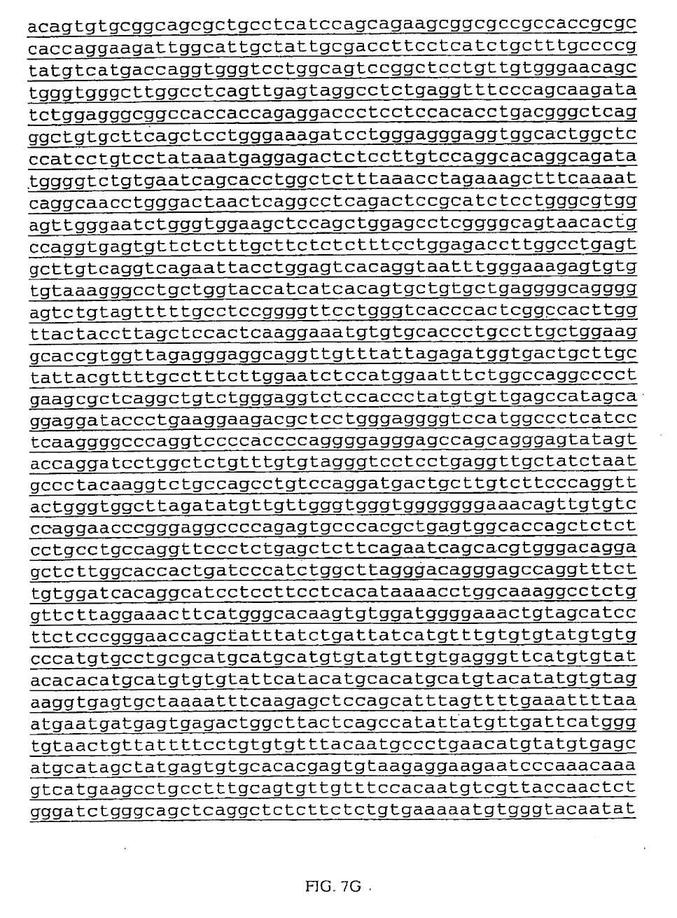

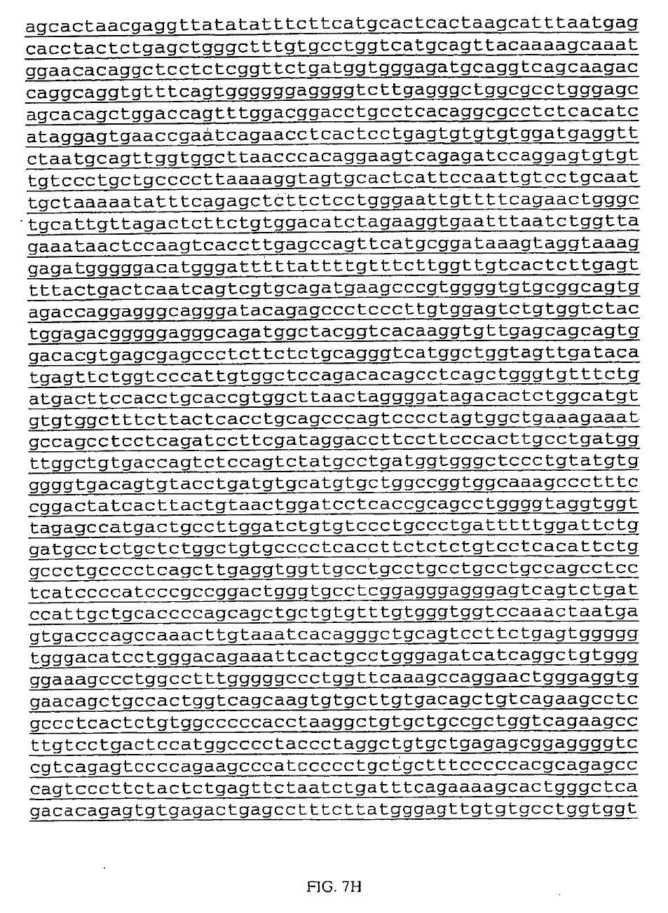

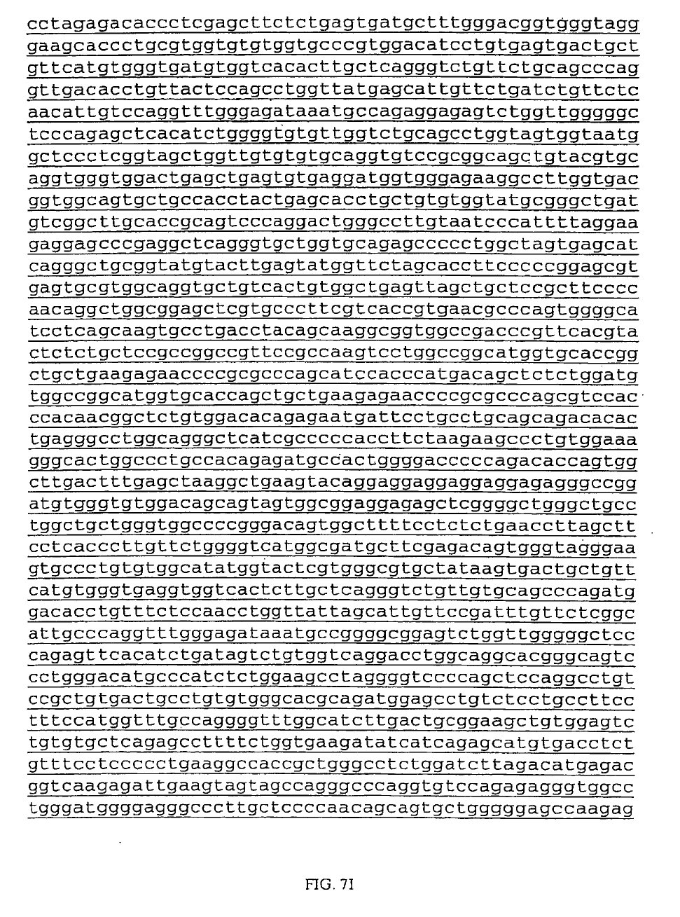

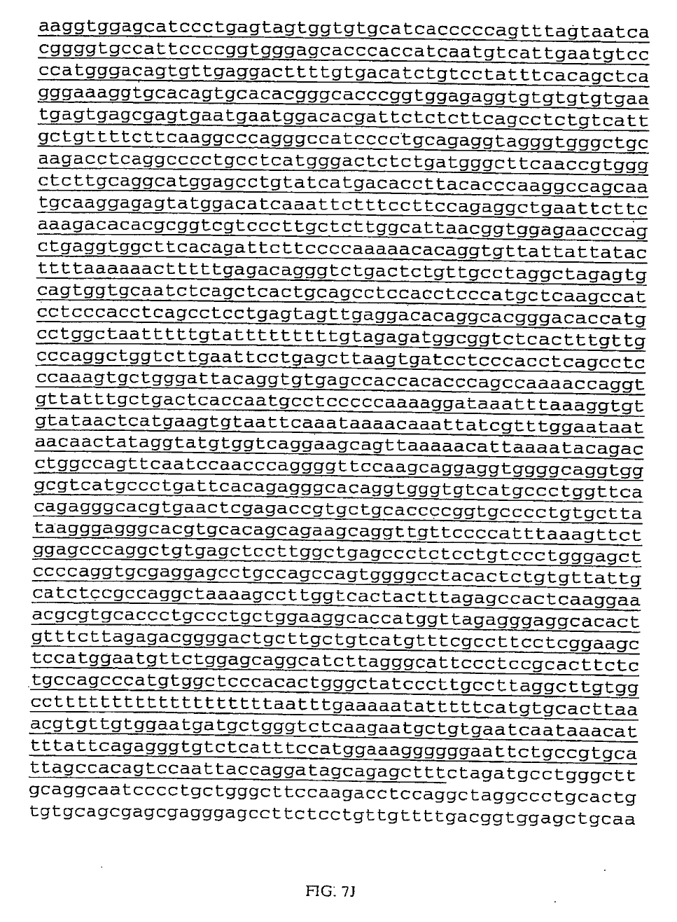

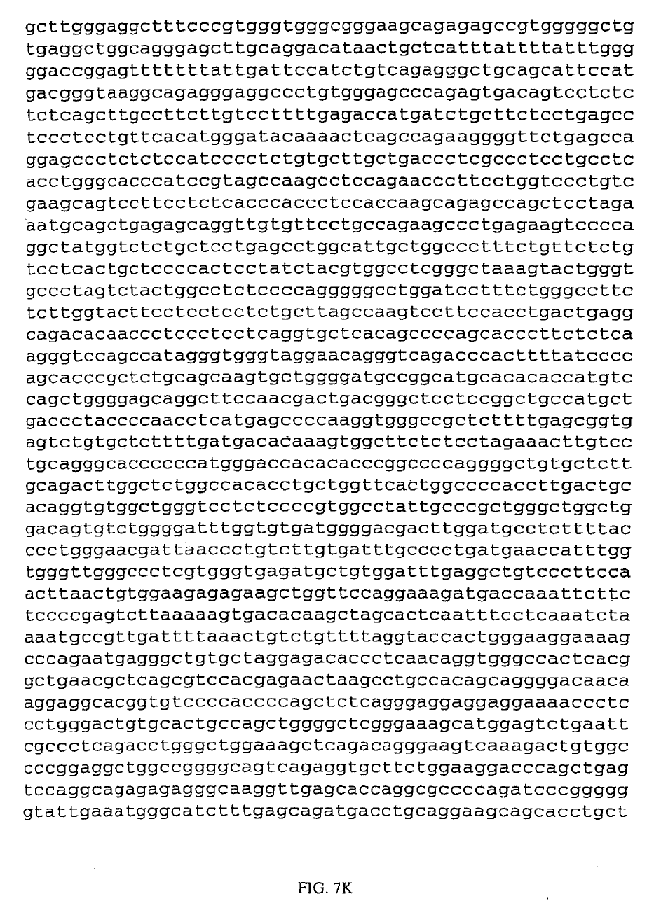

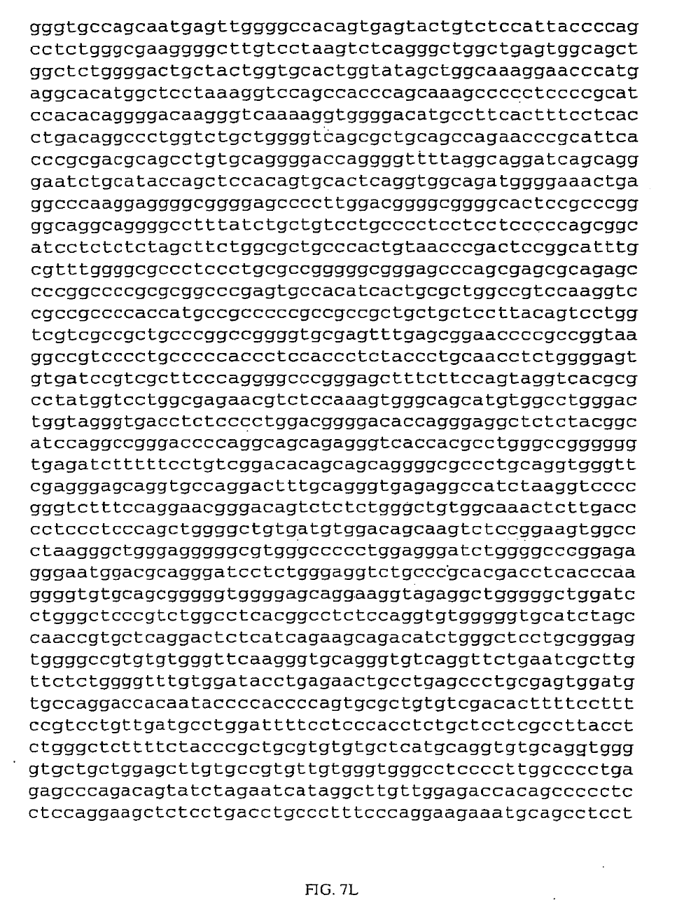

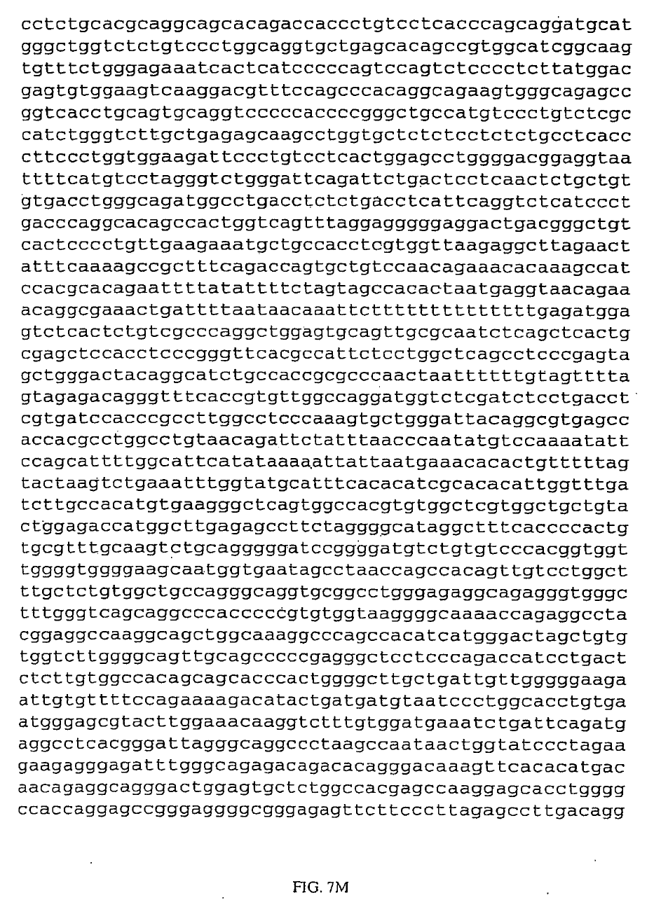

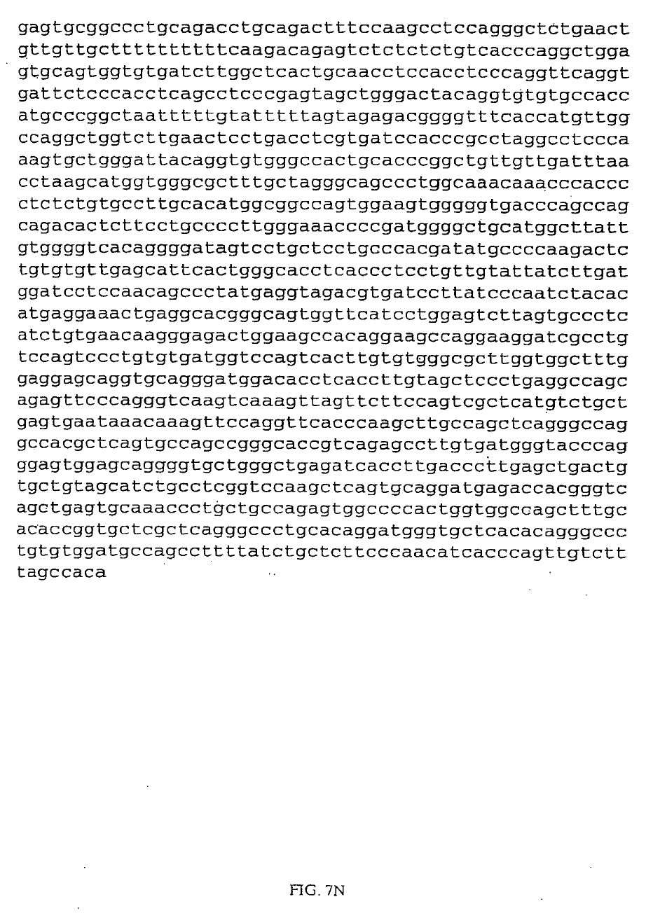

A TBLASTN search using the sequence of 2882 identified a human genomic sequence, deposited in the database May 7, 1999, which encodes the hCAR gene. This sequence corresponds to a 200 kb BAC clone designated AC007104, which has been mapped to chromosome 4 as of the March 2001 draft of the human genome of human chromosome 4 and contains a 666 bp uninterrupted stretch of homology to 2882 (bases 195068-195733—FIG. 7). A total of 3 stretches of homology were seen on the BLAST search, and these in combination with the genomic sequence were used to construct a contig for hCAR. This sequence was used to design oligonucleotide primers used in obtaining a physical clone. A physical cDNA clone, 2882h_{—}7N, was isolated from a human cerebellum library as described below. This clone contained a 5665 bp insert, incorporating 1.9 Kb 5′UT, a 1092 bp open reading frame, and 2.7 Kb 3′UT (FIG. 2).

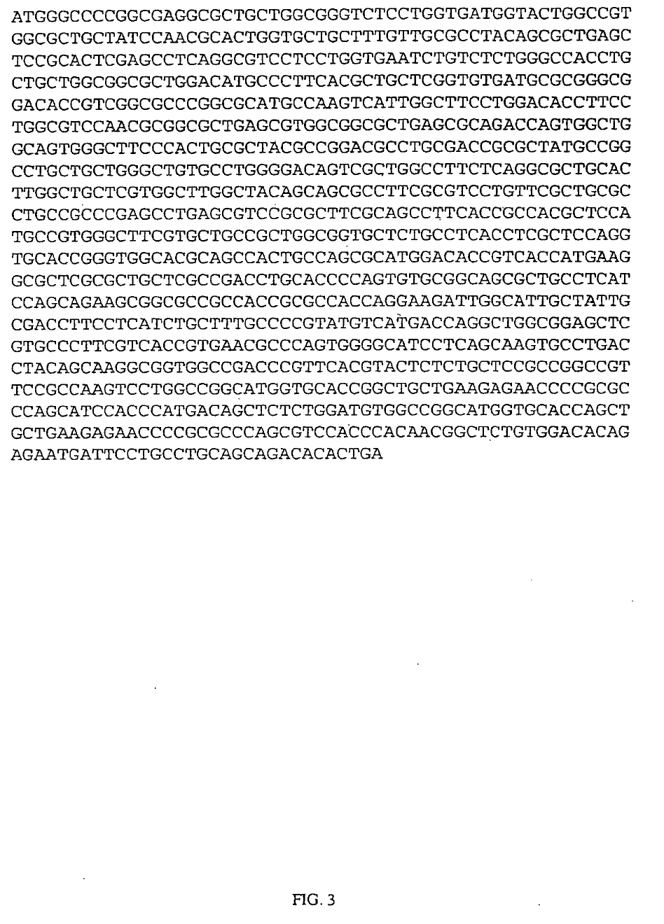

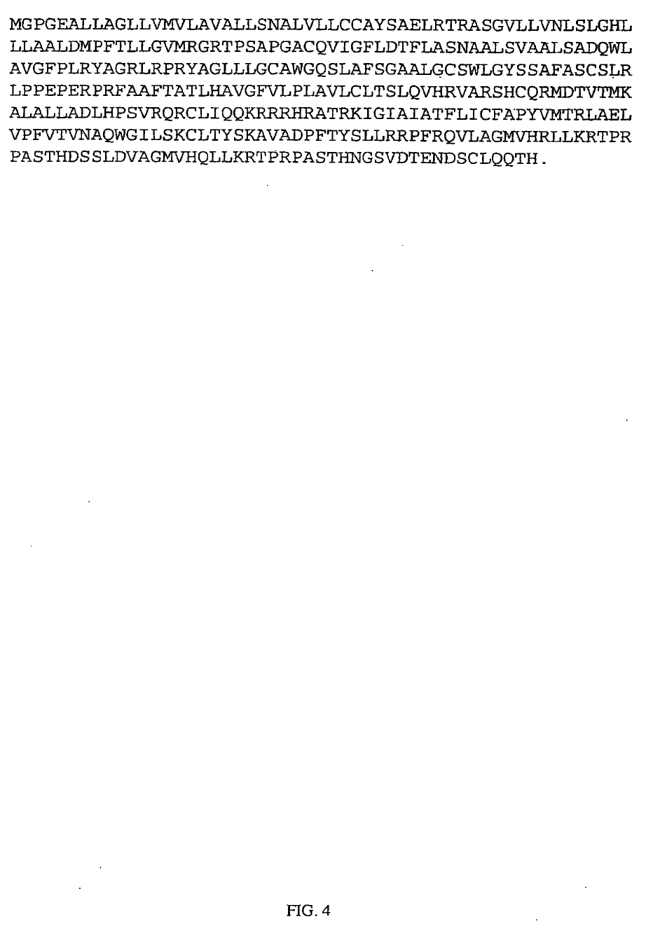

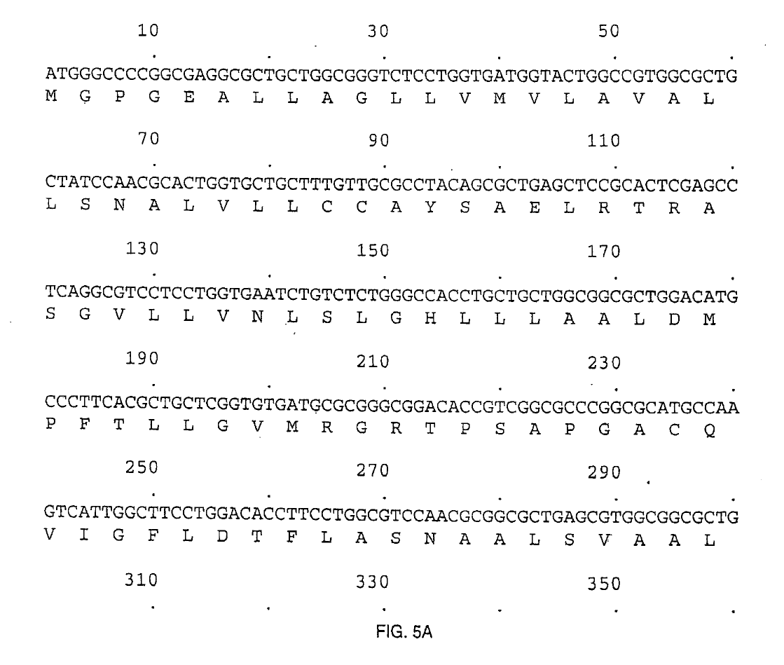

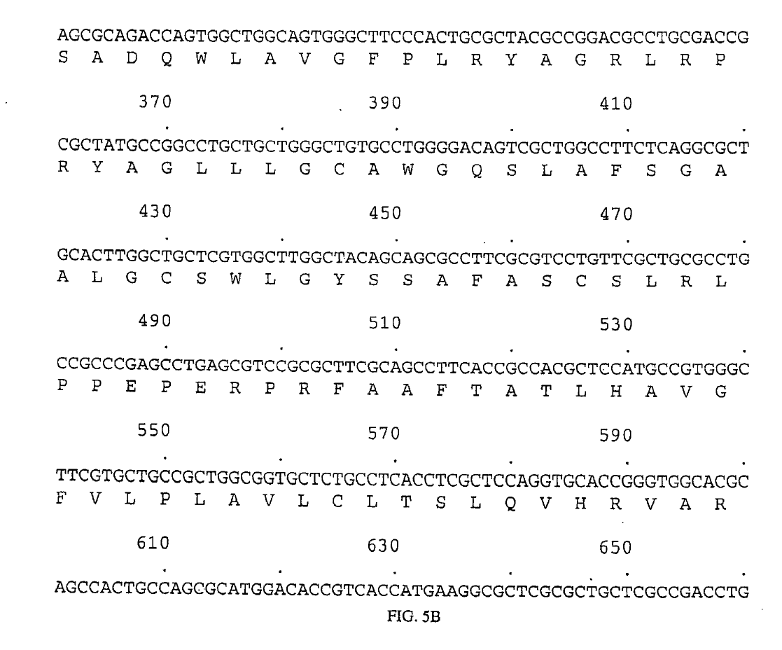

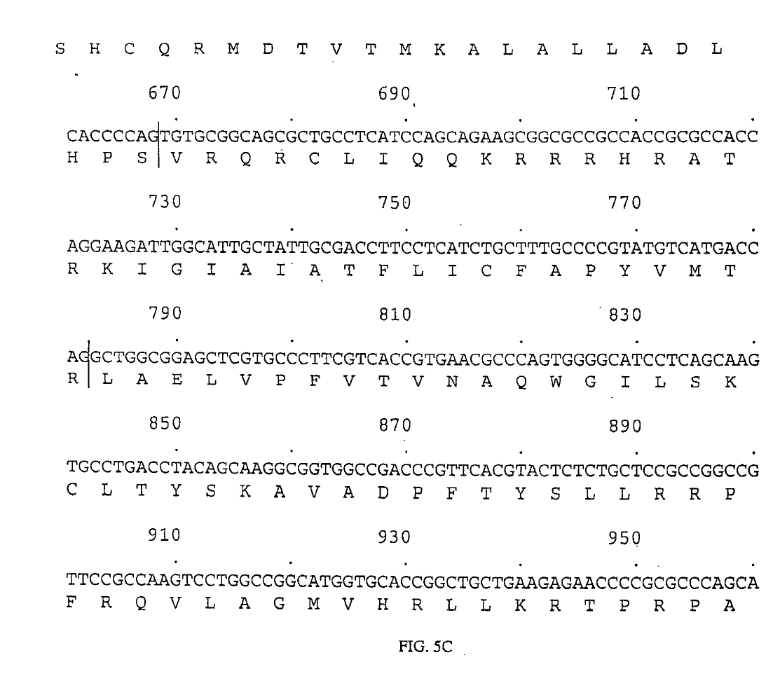

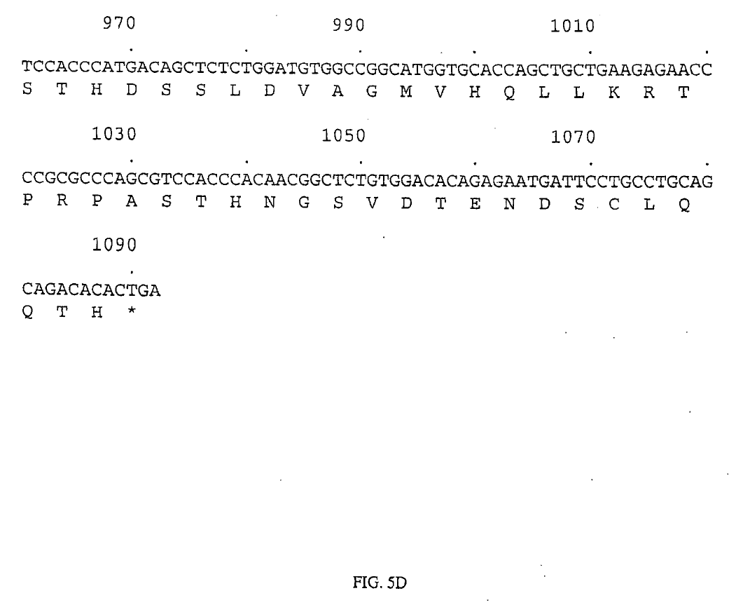

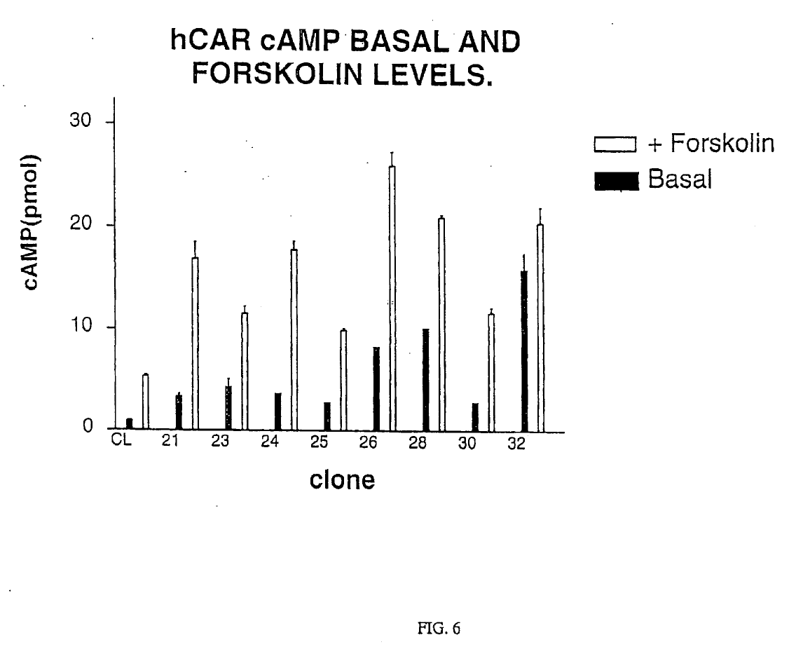

The conceptual translation (FIG. 3) of the 2882 homologous cDNA sequence is 58% similar (52% identical) to the protein sequence of 2882. A BLAST search of the 2882 homolog conceptual translation revealed weak similarity to galanin, histamine and somatostatin receptors. This gene, termed hCAR, represents the closest database homolog to 2882, and based on sequence homology, encodes a member of the G protein coupled receptor family.

### Example 2

### Methods Used in Cloning hCAR

### Library Construction

A plasmid cDNA library, designated L602C, was constructed using Clontech Human Brain, Cerebellum PolyA RNA (catalog #6543-1, lot no. 8070047) and Life Technologies SuperScript Plasmid System for cDNA Synthesis and Plasmid Cloning kit (catalog no. 18248-013). The manufacturer's protocol was followed with three modifications: 1) In both first and second strand synthesis reactions, DEPC-treated water was substituted for (alpha ³²P)dCTP. 2) The Sal I-adapted cDNA was size-fractionated by gel electrophoresis on 1% agarose, 0.1 ug/ml ethidium bromide, 1×TAE gels. The ethidium bromide-stained cDNA ≧3.0 kb was excised from the gel. The cDNA was purified from the agarose gel by electroelution (ISCO Little Blue Tank Electroelutor and protocol). 3) The gel-purified, size-fractionated Sal I-adapted cDNA was ligated to NotI-SalI digested pCMV-SPORT6 (Life Technologies, Inc.)

DNA from en masse plating of primary transformants of the library was obtained as follows. Ligated cDNA was used to transform electrocompetent E. coli cells (ElectroMAX DH10B cells and protocol, Life Technologies catalog no. 18290-015, Biorad E. coli pulser, voltage 1.8 KV, 3-5 msec pulse). The transformed cells were plated on LB-ampicillin agar plates and incubated overnight at 37° C. Approximately 10⁶ colony forming units (cfu) were plated at a density of 50,000 cfu/150 mm plate. Cells were washed off the plates with LB media (Maniatis, et al. 1982), and collected by centrifugation. Plasmid DNA was isolated from the cells using the QIAGEN Plasmid Giga Kit and protocol (catalog no. 12191).

### Plasmid PT 2C_B Construction

Plasmid pT_{—}2C_B, which contains a partial sequence of the predicted hCAR gene, was constructed as described below.

Polymerase chain reaction (PCR) amplification was performed using standard techniques. A reaction mixture was complied with components at the following final concentrations: 100 ng of DNA from en masse plated library L602C; 10 pmol of forward primer (5′GCCGTGGCGCTGCTATCCAACGCACTG, nt 1940-1966 FIG. 2 SEQ ID NO: 1); 10 pmol of reverse primer (5′ TCACACCGAGCAGCGTGAAGGGCAT, reverse complement of nt 2069-2093 FIG. 2 SEQ ID NO: 1); 0.2 mM each dATP, dTTP, dCTP, and dGTP (Amersham Pharmacia Biotech catalog no. 27-2094-01); 1.5 units Taq DNA polymerase; 1×PCR reaction buffer (Roche Molecular Biochemicals, catalog no. 1-596-594; 10 mM Tris-HCl; 1.5 mM MgCl₂, 50 mM KCl, pH8.3). The mixture was incubated at 94° C. for one minute, followed by 35 cycles of 94° C. 30 seconds, 72° C. 40 seconds, followed by a final incubation at 72° C. for five minutes (MJResearch DNA Engine Tetrad PTC-225).

The PCR reaction products (“DNA”) were size-fractionated by gel electrophoresis on 2% agarose, 0.1 ug/ml ethidium bromide, 1×TAE gels. The ethidium bromide-stained DNA band of the appropriate size (˜150 bp) was excised from the agarose gel. The DNA was extracted from the agarose using the Clonetech NucleoSpin Nucleic Acid Purification Kit (catalog no. K3051-2) and manufacturer's protocol. Subsequently, the DNA was sub-cloned into the vector pCRII-TOPO using the Invitrogen TOPO TA Cloning kit (Invitrogen catalog no. K4600) and manufacturer's protocol with modifications. Briefly, approximately 40 ng of the gel purified PCR product was incubated with one ul of the manufacturer supplied pCRII-TOPO DNA (10 ng/ul), and one ul of diluted Salt Solution (0.3M NaCl, 0.15M MgCl₂) in a final volume of six ul. The mixture was incubated for five minutes at room temperature (˜25° C.). Two uls of this reaction was added to electrocompetent cells (ElectroMAX DH10B cells, Life Technologies catalog no. 18290-015) and electroporated using the Biorad E. coli pulser (voltage 1.8 KV, 3-5 msec pulse). One ml of SOC (Sambrook et al, 1989) was added to the cells and the mixture incubated at 37° C. for 1.5 hours. The mixture was plated on LB-ampicillin agar plates and incubated overnight at 37° C. Bacterial clones containing the partial hCAR sequence (nt. 1940-2093 FIG. 2 SEQ ID NO: 1) in the pCRII-TOPO were identified by restriction digestion analysis and sequence analysis (ABI Prism BigDye Terminator Cycle Sequencing, catalog no. 4303154, ABI 377 instruments) of plasmid DNA prepared from isolated colonies. Plasmid DNA was prepared using the QIAprep Spin Miniprep Kit and protocol (Qiagen Inc, catalog no. 27106). One such bacterial clone was chosen and designated pT_{—}2C_B.

Isolation of Clone 2882h_{—}7N

cDNA clone 2882h_{—}7N was isolated by screening approximately 500,000 primary transformants from plasmid cDNA library L602C with a 32-P-labeled DNA probe using standard molecular biology techniques. Probe generation is described below. Plasmid DNA, prepared as described above, from isolated positively hybridizing colonies from L602C was analyzed by restriction digestion analysis and sequence analysis (ABI Prism BigDye Terminator Cycle Sequencing, catalog no. 4303154, ABI 377 instruments). cDNA clone 2882h_{—}7N, isolated Jun. 22, 2000, contained the predicted hCAR open reading frame.

### Probe Generation

The hCAR specific probe used in the library screen was generated as follows. Plasmid DNA from pT_{—}2C_B was restriction digested with EcoRI (New England Biolabs, catalog no. R0101 L) according to the manufacturers protocol. Restriction fragments were size-fractionated by gel electrophoresis on 1.5% agarose, 0.1 ug/ml ethidium bromide, 1×TAE gels. The ethidium bromide-stained DNA band of the appropriate size (˜150 bp) was excised from the agarose gel. Next, the DNA was extracted from the agarose using the Clonetech NucleoSpin Nucleic Acid Purification Kit (catalog no. K3051-2) and manufacturer's protocol. The extracted DNA was labeled with Redivue (alpha ³²P)dCTP (Amersham Pharmacia, catalog no. AA0005) using the Prime-It II Random Primer Labeling Kit and protocol (Stratagene, catalog no. 300385). Un-incorporated (alpha ³²P)dCTP was removed with Amersham's NICK column and protocol (catalog no. 17-0855-02)

### Example 3

### Tissue Expression of the hCAR Gene

To assess the tissue distribution of the hCAR transcript, Northern analysis was performed using blots containing 1 ug of poly A+ RNA per lane isolated from various human tissues (catalog no. 7780-1, Clontech, Palo Alto, Calif.) and probed with a human hCAR-specific probe. The filters were prehybridized in 10 ml of Express Hyb hybridization solution (Clontech, Palo Alto, Calif.) at 68 C for 1 hour, after which 100 ng of 32p labeled probe was added. The probe was generated using the Stratagene Prime-It kit, Catalog Number 300392 (Clontech, Palo Alto, Calif.).

The hCAR specific 32-P-labeled DNA probe contained nucleotides-2282-2782 of the hCAR cDNA sequence (FIG. 2, SEQ ID NO: 1). A single ˜5.0 kb transcript was detectable in placental and whole brain tissue on the Human 12−Lane Multiple Tissue Northern. A transcript was not detected in other tissues on this Northern. The expression of hCAR was further analyzed with Human Multiple Tissue Expression Array (catalog no. 7775-1, user manual PT3307-1) membranes. Hybridization to poly(A)+ RNA from multiple tissues was detectable on the Human Multiple Tissue Expression Array: strong hybridization to placenta, fetal brain, whole brain, cerebral cortex, frontal lobe, parietal lobe, occipital lobe, temporal lobe, paracentral gyrus of cerebral cortex, pons, left and right cerebellum, corpus callosum, amygdala, caudate nucleus, hippocampus, medulla oblongata, putamen, substantia nigra, accumbens nucleus, thalamus, pituitary gland and spinal cord. Weak hybridization, potentially non-specific background hybridization, was seen in numerous tissues: heart, intestinal tract, kidney, spleen, thymus, skeletal muscle, lymph node, bone marrow, trachea, lung, liver, pancreas, bladder, uterus, prostate, testis, ovary, adrenal gland, thyroid gland, salivary, gland, and mammary gland.

### Example 4

### Expression of Recombinant hCAR Protein in Bacterial Cells

In this example, hCAR is expressed as a recombinant glutathione-S-transferase (GST) fusion protein in E. coli and the fusion protein is isolated and characterized.

Specifically, hCAR is fused to GST and this fusion protein is expressed in E. coli, e.g., strain PEB 199. As the human protein is predicted to be approximately 39 kDa, and GST is predicted to be 26 kDa, the fusion protein is predicted to be approximately 65 kDa, in molecular weight. Expression of the GST-hCAR fusion protein in PEB199 is induced with IPTG. The recombinant fusion protein is purified from crude bacterial lysates of the induced PEB 199 strain by affinity chromatography on glutathione beads.

Using polyacrylamide gel electrophoretic analysis of the protein purified from the bacterial lysates, the molecular weight of the resultant fusion protein may be determined.

### Example 5

### Expression of Recombinant hCAR Protein in COS Cells

To express the hCAR gene in COS cells, the pcDNA/Amp vector by Invitrogen Corporation (San Diego, Calif.) maybe used. This vector contains an SV40 origin of replication, an ampicillin resistance gene, an E. coli replication origin, a CMV promoter followed by a polylinker region, and an SV40 intron and polyadenylation site. A DNA fragment encoding the entire hCAR protein and a HA tag (Wilson et al. (1984) Cell 37:767) fused in-frame to the 3′ end of the fragment is cloned into the polylinker region of the vector, thereby placing the expression of the recombinant protein under the control of the CMV promoter.

To construct the plasmid, the hCAR DNA sequence is amplified by PCR using two primers. The 5′ primer contains the restriction site of interest followed by approximately twenty nucleotides of the hCAR coding sequence starting from the initiation codon; the 3′ end sequence contains complementary sequences to the other restriction site of interest, a translation stop codon, the HA tag and the last 20 nucleotides of the hCAR coding sequence. The PCR amplified fragment and the pcDNA/Amp vector are digested with the appropriate restriction enzymes and the vector is dephosphorylated using the CIAP enzyme (New England Biolabs, Beverly, Mass.). Preferably the two restriction sites chosen are different so that the hCAR gene is inserted in the correct orientation. The ligation mixture is transformed into E. coli cells (strains HB101, DH5a, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the hCAR-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook, J., Fritsh, E. F., and Maniatis, T. Molecular Cloning: A Laboratory Manual. 2nd, ed, Cold Spring Harbor Laboratory, Cold Spring Harbor Laboratory Press, Cold Spring Harbor, N.Y., 1989. The expression of the hCAR protein is detected by radiolabelling (35S-methionine or 35S-cysteine available from NEN, Boston, Mass., can be used) and immunoprecipitation (Harlow, E. and Lane, D. Antibodies: A Laboratory Manual, Cold Spring Harbor Laboratory Press, Cold Spring Harbor, N.Y., 1988) using an HA specific monoclonal antibody. Briefly, the cells are labelled for 8 hours with 35S-methionine (or 35S-cysteine). The culture media are then collected and the cells are lysed using detergents (RIPA buffer, 150 mM NaCl, l % NP-40, 0.1% SDS, 0.5% DOC, 50 mM Tris, pH 7.5). Both the cell lysate and the culture media are precipitated with an HA specific monoclonal antibody. Precipitated proteins are then analyzed by SDS-PAGE.

Alternatively, DNA containing the hCAR coding sequence is cloned directly into the polylinker of the pcDNA/Amp vector using the appropriate restriction sites.

The resulting plasmid is transfected into COS cells in the manner described above, and the expression of the hCAR protein is detected by radiolabelling and immunoprecipitation using an hCAR specific monoclonal antibody.

### Example 5

### Expression of hCAR in Mammalian Cells

### Cell Line Generation

The open reading frame of hCAR was ligated into the mammalian expression vector pcDNA3.1+zeo (Invitrogen, 1600 Faraday Avenue, Carlsbad, Calif. 92008). HEK 293 cells were transfected with the plasmid and selected with 500 μg/ml zeocin. Zeocin resistant clones were tested for expression of hCAR by RT-PCR and then tested for their ability to stimulate cAMP production.

### Cyclase Assays

4×10⁵ cells were plated into 96 well Biocoat cell culture plates (Becton Dickinson, 1 Becton Drive, Franklin Lakes, N.J. 07417-1886) 24 hours prior to assay. The cells were then incubated in Krebs-bicarbonate buffer at 37° C. for 15 minutes. A 5 minute pretreatment with 500 μM isobutylmethyl xanthine (IBMX) preceded a 12 minute stimulation with 1 μM forskolin or buffer for determination of basal cAMP levels. cAMP levels were determined using the SPA assay (Amersham Pharmacia Biotech, 800 Centennial Avenue, Pistcataway, N.J. 08855).

### Results

Transfection of HEK 293 cells with the hCAR mammalian expression vector results in increased basal levels of cAMP when compared to the control (CL) line. The increase ranges from 3 fold to 16 fold. The increased basal levels in the absence of agonist is termed constitutive activity and is the result of the hCAR stimulating the cAMP synthesis pathway without the need to be activated by a ligand. The levels of cAMP can be further increased with forskolin. The stimulated amounts of cAMP are again greater than those seen with the control line (5 pMol) and range from 9 to 23 pMols cAMP.

### Example 6

### Characterization of the Human hCAR Protein

In this example, the amino acid sequence of the human hCAR protein was compared to amino acid sequences of known proteins and various motifs were identified.

The human hCAR protein, the amino acid sequence of which is shown in FIG. 3 (SEQ ID NO: 2), is a protein which includes 363 amino acid residues.

Hydrophobicity analysis indicated that the human hCAR protein contains the expected 7 transmembrane domains and that they are located at amino acid residues: 47-62; 80-97; 100-103; 129-153; 175-190; 248-258; and 272-274.

### Example 7

### Construction of hCAR Gene Targeting Vector

A partial murine hCAR cDNA clone is isolated from a mouse brain cDNA library (obtained commercially from Stratagene) using the full length human hCAR coding sequence as a probe by standard techniques. The murine hCAR cDNA is then used as a probe to screen a genomic DNA library made from the 129 strain of mouse, again using standard techniques. The isolated murine hCAR genomic clones are then subcloned into a plasmid vector, pBluescript (obtained commercially from Stratagene), for restriction mapping, partial DNA sequencing, and construction of the targeting vector. To functionally disrupt the hCAR gene, a targeting vector may be prepared in which non-homologous DNA is inserted within the first coding exon, deleting the start codon and about 600 bp of hCAR coding sequence (which would include the first 5 transmembrane domains) in the process and rendering the remaining downstream hCAR coding sequences out of frame with respect to the start of translation. Therefore, if any translation products were to be formed from alternately spliced transcripts of the hCAR gene, they would not contain all 7 transmembrane domains required for normal function of a GPCR. The hCAR targeting vector is constructed using standard molecular cloning techniques. The targeting vector would contain 1-5 kb of murine hCAR genomic sequence upstream of the initiating codon immediately followed by the neomycin phosphotransferase (neo) gene under the control of the phosphoglycerokinase promoter. Immediately downstream of the neomycin cassette is 1-5 kb of murine hCAR genomic sequence corresponding to a region approximately 2 kb downstream of the murine hCAR start codon. This is followed by the herpes simplex thymidine kinase (HSV tk) gene under the control of the phosphoglycerokinase promoter. The upstream and downstream genomic cassettes in this vector are in the same 5′ to 3′ orientation as the endogenous murine gene. The positive selection neo gene replaces the first coding exon of the hCAR sequences and in the opposite orientation as the hCAR gene, whereas the negative selection HSV tk gene is at the 3′ end of the construct. This configuration allowed for the use of the positive and negative selection approach for homologous recombination (Mansour, S. L. et al. (1988) Nature 336:348). Prior to transfection into embryonal stem cells, the plasmid is linearized by restriction enzyme digestion.

### Example 8

### Transfection and Analysis of Embryonal Stem Cells

Embryonic stem cells (For example, strain D3, Doestschman, T. C. et al. (1985) J. Embryol. Exp. Morphol. 87:27-45) are cultured on a neomycin resistant embryonal fibroblast feeder layer grown in Dulbecco's Modified Eagles medium supplemented with 15% Fetal Calf Serum, 2 mM glutamine, penicillin (50 u/ml)/streptomycin (50 u/ml), non-essential amino acids, 100 uM 2-mercaptoethanol and 500 u/ml leukemia inhibitory factor. Medium is changed daily and cells are subcultured every two to three days and are then transfected with linearized plasmid by electroporation (25 uF capacitance and 400 Volts). The transfected cells are cultured in non-selective media for 1-2 days post transfection. Subsequently, they are cultured in media containing gancyclovir and neomycin for 5 days, of which the last 3 days are in neomycin alone. After expanding the clones, an aliquot of cells is frozen in liquid nitrogen. DNA is prepared from the remainder of cells for genomic DNA analysis to identify clones in which homologous recombination had occurred between the endogenous hCAR gene and the targeting construct. To prepare genomic DNA, ES cell clones are lysed in 100 mM Tris HCl, pH 8.5, 5 mM EDTA, 0.2% SDS, 200 mM NaCl and 100 .mu.g of proteinase K/ml. DNA is recovered by isopropanol precipitation, solubilized in 10 mM Tris HCl, pH 8.0/0.1 mM EDTA. To identify homologous recombinant clones, genomic DNA isolated from the clones is digested with restriction enzymes. After restriction digestion, the DNA can be resolved on a 0.8% agarose gel, blotted onto a Hybond N membrane and hybridized at 65° C. with probes that bind a region of the hCAR gene proximal to the 5′ end of the targeting vector and probes that bind a region of the hCAR gene distal to the 3′ end of the targeting vector. After standard hybridization, the blots are washed with 40 mM NaPO4 (pH 7.2), 1 mM EDTA and 1% SDS at 65° C. and exposed to X_ray film. Hybridization of the 5′ probe to the wild type hCAR allele results in a fragment readily discernible by autoradiography from the mutant hCAR allele having the neo insertion.

### Example 9

### Generation of hCAR Deficient Mice

Female and male mice are mated and blastocysts are isolated at 3.5 days of gestation. 10 to 12 cells from the clone described in Example 2 are injected per blastocyst and 7 or 8 blastocysts are transferred to the uterus of a pseudopregnant female. Pups are delivered by cesarean section on the 18th day of gestation and placed with a foster BALB/c mother. Resulting male and female chimeras are mated with female and male BALB/C mice (non-pigmented coat), respectively, and germline transmission is determined by the pigmented coat color derived from passage of 129 ES cell genome through the germline. The pigmented heterozygotes are likely to carry the disrupted hCAR allele and therefore these animals are mated and, Mendelian genetics predicts that approximately 25% of the offspring will be homozygous for the hCAR null mutation. Genotyping of the animals is accomplished by obtaining tail genomic DNA.

To confirm that the hCAR −/− mice do not express full-length hCAR mRNA transcripts, RNA is isolated from various tissues and analyzed by standard Northern hybridizations with an hCAR cDNA probe or by reverse transcriptase-polymerase chain reaction (RT-PCR). RNA is extracted from various organs of the mice using 4M Guanidinium thiocyanate followed by centrifugation through 5.7 M CsCl as described in Sambrook et al. (Molecular Cloning: A Laboratory Manual, 2nd Edition, Cold Spring Harbor Laboratory press (1989)). Northern analysis of hCAR mRNA expression in brain or placenta will demonstrate that the full-length hCAR mRNA is not detectable in brain or placenta from hCAR −/− mice. Primers specific for the neomycin gene will detect a transcript in hCAR +/− and −/− but not +/+ animals. Northern and RT-PCT analyses are used to confirm that homozygous disruption of the hCAR gene results in the absence of detectable full-length hCAR mRNA transcripts in the hCAR −/− mice. To examine hCAR protein expression in the hCAR deficient mice, Western blot analyses are performed on lysates from isolated tissue, including brain and placenta using standard techniques. These results will confirm that homozygous disruption of the hCAR gene results in an absence of detectable hCAR protein in the −/− mice.

### Example 10

### Inhibition of hCAR Production

### Design of RNA Molecules as Compositions of the Invention

All RNA molecules in this experiment are approximately 600 nts in length, and all RNA molecules are designed to be incapable of producing functional hCAR protein. The molecules have no cap and no poly-A sequence; the native initiation codon is not present, and the RNA does not encode the full-length product. The following RNA molecules are designed:

(1) a single-stranded (ss) sense RNA polynucleotide sequence homologous to a portion of hCAR murine messenger RNA (m.RNA);

(2) a ss anti-sense RNA polynucleotide sequence complementary to a portion of hCAR murine mRNA,

(3) a double-stranded (ds) RNA molecule comprised of both sense and anti-sense a portion of hCAR murine mRNA polynucleotide sequences,

(4) a ss sense RNA polynucleotide sequence homologous to a portion of hCAR murine heterogeneous RNA (hnRNA),

(5) a ss anti-sense RNA polynucleotide sequence complementary to a portion of hCAR murine hnRNA,

(6) a ds RNA molecule comprised of the sense and anti-sense hCAR murine hnRNA polynucleotide sequences,

(7) a ss murine RNA polynucleotide sequence homologous to the top strand of the a portion of hCAR promoter,

(8) a ss murine RNA polynucleotide sequence homologous to the bottom strand of the a portion of hCAR promoter, and

(9) a ds RNA molecule comprised of murine RNA polynucleotide sequences homologous to the top and bottom strands of the hCAR promoter.

The various RNA molecules of (1)-(9) above may be generated through T7 RNA polymerase transcription of PCR products bearing a T7 promoter at one end. In the instance where a sense RNA is desired, a T7 promoter is located at the 5′ end of the forward PCR primer. In the instance where an antisense RNA is desired, the T7 promoter is located at the 5′ end of the reverse PCR primer. When dsRNA is desired both types of PCR products may be included in the T7 transcription reaction. Alternatively, sense and anti-sense RNA may be mixed together after transcription, under annealing conditions, to form ds RNA.

### Construction of Expression Plasmid Encoding a Fold-Back Type of RNA

An expression plasmid encoding an inverted repeat of a portion of the hCAR gene may be constructed using the information disclosed in this application. A DNA fragment encoding an hCAR foldback transcript may be prepared by PCR amplification and introduced into suitable restriction sites of a vector which includes the elements required for transcription of the hCAR foldback transcript. The DNA fragment would encode a transcript that contains a fragment of the hCAR gene of approximately at least 600 nucleotides in length, followed by spacer sequence of at least 10 bp but not more than 200 bp, followed by the reverse complement of the hCAR sequence chosen. CHO cells transfected with the construct will produce only fold-back RNA in which complementary target gene sequences form a double helix.

### Assay

Balb/c mice (5 mice/group) may be injected intercranially with the murine hCAR chain specific RNAs described above or with controls at doses ranging between 10 μg and 500 μg. Brains are harvested from a sample of the mice every four days for a period of three weeks and assayed for hCAR levels using the antibodies as disclosed herein or by northern blot analysis for reduced RNA levels.

According to the present invention, mice receiving ds RNA molecules derived from both the hCAR mRNA, hCAR hnRNA and ds RNA derived from the hCAR promoter demonstrate a reduction or inhibition in hCAR production. A modest, if any, inhibitory effect is observed in sera of mice receiving the single stranded hCAR derived RNA molecules, unless the RNA molecules have the capability of forming some level of double-strandedness.

### Example 11

### Method of the Invention in the Prophylaxis of Disease

### In Vivo Assay

Using the hCAR specific RNA molecules described in Example 10, which do not have the ability to make hCAR protein and hCAR specific RNA molecules as controls, mice may be evaluated for protection from hCAR related disease through the use of the injected hCAR specific RNA molecules of the invention.

Balb/c mice (5 mice/group) may be immunized by intercranial injection with the described RNA molecules at doses ranging between 10 and 500 μg RNA. At days 1, 2, 4 and 7 following RNA injection, the mice may be observed for signs of hCAR related phenotypic change.

According to the present invention, because the mice that receive dsRNA molecules of the present invention which contain the hCAR sequence may be shown to be protected against hCAR related disease. The mice receiving the control RNA molecules may be not protected. Mice receiving the ss RNA molecules which contain the hCAR sequence may be expected to be minimally, if at all, protected, unless these molecules have the ability to become at least partially double stranded in vivo.

According to this invention, because the dsRNA molecules of the invention do not have the ability to make hCAR protein, the protection provided by delivery of the RNA molecules to the animal is due to a non-immune mediated mechanism that is gene specific.

### Example 12

### RNA Interference in Drosophila and Chinese Hamster Cultured Cells

To observe the effects of RNA interference, either cell lines naturally expressing hCAR can be identified and used or cell lines which express hCAR as a transgene can be constructed by well known methods (and as outlined herein). As examples, the use of Drosophila and CHO cells are described. Drosophila S2 cells and Chinese hamster CHO-K1 cells, respectively, may be cultured in Schneider medium (Gibco BRL) at 25° C. and in Dulbecco's modified Eagle's medium (Gibco BRL) at 37° C. Both media may be supplemented with 10% heat-inactivated fetal bovine serum (Mitsubishi Kasei) and antibiotics (10 units/ml of penicillin (Meiji) and 50 μg/ml of streptomycin (Meiji)).

### Transfection and RNAi Activity Assay

S2 and CHO-K1 cells, respectively, are inoculated at 1×10⁶ and 3×10⁵ cells/ml in each well of 24-well plate. After 1 day, using the calcium phosphate precipitation method, cells are transfected with hCAR dsRNA (80 pg to 3 μg). Cells may be harvested 20 h after transfection and hCAR gene expression measured.

### Example 13

### Antisense Inhibition in Vertebrate Cell Lines

Antisense can be performed using standard techniques including the use of kits such as those of Sequitur Inc. (Natick, Mass.). The following procedure utilizes phosphorothioate oligodeoxynucleotides and cationic lipids. The oligomers are selected to be complementary to the 5′ end of the mRNA so that the translation start site is encompassed.

1) Prior to plating the cells, the walls of the plate are gelatin coated to promote adhesion by incubating 0.2% sterile filtered gelatin for 30 minutes and then washing once with PBS. Cells are grown to 40-80% confluence. Hela cells can be used as a positive control.

2) the cells are washed with serum free media (such as Opti-MEMA from Gibco-BRL).

3) Suitable cationic lipids (such as Oligofectibn A from Sequitur, Inc.) are mixed and added to serum free media without antibiotics in a polystyrene tube. The concentration of the lipids can be varied depending on their source. Add oligomers to the tubes containing serum free media/cationic lipids to a final concentration of approximately 200 nM (50-400 nM range) from a 100 μM stock (2 μl per ml) and mix by inverting.

4) The oligomer/media/cationic lipid solution is added to the cells (approximately 0.5 mls for each well of a 24 well plate) and incubated at 37° C. for 4 hours.

5) The cells are gently washed with media and complete growth media is added. The cells are grown for 24 hours. A certain percentage of the cells may lift off the plate or become lysed.

Cells are harvested and hCAR gene expression is measured.

### Example 14

### Production of Transfected Cell Strains by Gene Targeting

Gene targeting occurs when transfecting DNA either integrates into or partially replaces chromosomal DNA sequences through a homologous recombinant event. While such events can occur in the course of any given transfection experiment, they are usually masked by a vast excess of events in which plasmid DNA integrates by nonhomologous, or illegitimate, recombination.

### Generation of a Construct Useful for Selection of Gene Targeting Events in Human Cells

One approach to selecting the targeted events is by genetic selection for the loss of a gene function due to the integration of transfecting DNA. The human HPRT locus encodes the enzyme hypoxanthine-phosphoribosyl transferase. Hprt-cells can be selected for by growth in medium containing the nucleoside analog 6-thioguanine (6-TG): cells with the wild-type (HPRT+) allele are killed by 6-TG, while cells with mutant (hprt−) alleles can survive. Cells harboring targeted events which disrupt HPRT gene function are therefore selectable in 6-TG medium.

To construct a plasmid for targeting to the HPRT locus, the 6.9 kb HindIII fragment extending from positions 11,960-18,869 in the HPRT sequence (Genebank name HUMHPRTB; Edwards, A. et al., Genomics 6:593-608 (1990)) and including exons 2 and 3 of the HPRT gene, may be subdcloned into the HindIII site of pUC12. The resulting clone is cleaved at the unique XhoI site in exon 3 of the HPRT gene fragment and the 1.1 kb SalI-XhoI fragment containing the neo gene from pMC1 Neo (Stratagene) is inserted, disrupting the coding sequence of exon 3. One orientation, with the direction of neo transcription opposite that of HPRT transcription was chosen and designated pE3Neo. The replacement of the normal HPRT exon 3 with the neo-disrupted version will result in an hprt−, 6-TG resistant phenotype. Such cells will also be G418 resistant.

### Generation of a Construct for Targeted Insertion of a Gene of Therapeutic Interest Into the Human Genome and its Use in Gene Targeting

A variant of pE3Neo, in which a hCAR gene is inserted within the HPRT coding region, adjacent to or near the neo gene, can be used to target the hCAR gene to a specific position in a recipient primary or secondary cell genome. Such a variant of pE3Neo can be constructed for targeting the hCAR gene to the HPRT locus.

A DNA fragment containing the hCAR gene and linked mouse metallothionein (mMT) promoter is constructed. Separately, pE3Neo is digested with an enzyme which cuts at the junction of the neo fragment and HPRT exon 3 (the 3′ junction of the insertion into exon 3). Linearized pE3Neo fragment may be ligated to the hCAR-mMT fragment.

Bacterial colonies derived transfection with the ligation mixture are screened by restriction enzyme analysis for a single copy insertion of the hCAR-mMT fragment. An insertional mutant in which the hCAR DNA is transcribed in the same direction as the neo gene is chosen and designated pE3Neo/hCAR. pE3Neo/hCAR is digested to release a fragment containing HPRT, neo and mMT-hCAR sequences. Digested DNA is treated and transfected into primary or secondary human fibroblasts. G418^{r} TGᵣ colonies are selected and analyzed for targeted insertion of the mMT-hCAR and neo sequences into the HPRT gene. Individual colonies may be assayed for hCAR expression using antibodies as described elsewhere herein.

Secondary human fibroblasts may be transfected with pE3Neo/hCAR and thioguanine-resistant colonies analyzed for stable hCAR expression and by restriction enzyme and Southern hybridization analysis.

The use of homologous recombination to target a hCAR gene to a specific position in a cell's genomic DNA can be expanded upon and made more useful for producing products for therapeutic purposes (e.g., pharmaceuticals, gene therapy) by the insertion of a gene through which cells containing amplified copies of the gene can be selected for by exposure of the cells to an appropriate drug selection regimen. For example, pE3neo/hCAR can be modified by inserting the dhfr, ada, or CAD gene at a position immediately adjacent to the hCAR or neo genes in pE3neo/hCAR. Primary, secondary, or immortalized cells are transfected with such a plasmid and correctly targeted events are identified. These cells are further treated with increasing concentrations of drugs appropriate for the selection of cells containing amplified genes (for dhfr, the selective agent is methotrexate, for CAD the selective agent is N-(phosphonacetyl)-L-aspartate (PALA), and for ada the selective agent is an adenine nucleoside (e.g., alanosine). In this manner the integration of the gene of therapeutic interest will be coamplified along with the gene for which amplified copies are selected. Thus, the genetic engineering of cells to produce genes for therapeutic uses can be readily controlled by preselecting the site at which the targeting construct integrates and at which the amplified copies reside in the amplified cells.

Construction of Targeting Plasmids for Placing the hCAR Gene Under the Control of The Mouse Metallothionein Promoter in Primary, Secondary and Immortalized Human Fibroblasts

The following serves to illustrate one embodiment of the present invention, in which the normal positive and negative regulatory sequences upstream of the hCAR gene are altered to allow expression of hCAR in primary, secondary or immortalized human fibroblasts or other cells which do not express hCAR in significant quantities.

Unique sequences of SEQ ID NO: 3 are selected which are located upstream from the hCAR coding region and ligated to the mouse metallothionein promoter as targeting sequences. Typically, the 1.8 kb EcoRI-BgIII from the mMT-I gene (containing no mMT coding sequences; Hamer, D. H. and Walling, M., J. Mol. Appl. Gen. 1:273-288 (1982); this fragment can also be isolated by known methods from mouse genomic DNA using PCR primers designed from analysis of mXT sequences available from Genbank; i.e., MUSMTI, MUSMTIP, MUSMTIPRM) is made blunt-ended by known methods and ligated with the 5′ hCAR sequences. The orientations of resulting clones are analyzed and suitable DNAs are used for targeting primary and secondary human fibroblasts or other cells which do not express hCAR in significant quantities.

Additional upstream sequences are useful in cases where it is desirable to modify, delete and/or replace negative regulatory elements or enhancers that lie upstream of the initial target sequence.

The cloning strategies described above allow sequences upstream of hCAR to be modified in vitro for subsequent targeted transfection of primary, secondary or immortalized human fibroblasts or other cells which do not express hCAR in significant quantities. The strategies describe simple insertions of the mMT promoter, and allow for deletion of the negative regulatory region, and deletion of the negative regulatory region and replacement with an enhancer with broad host-cell activity.

Targeting to Sequences Flanking the hCAR Gene and Isolation of Targeted Primary. Secondary and Immortalized Human Fibroblasts by Screening

Targeting fragment containing the mMT promoter and hCAR upstream sequences may be purified by phenol extraction and ethanol precipitation and transfected into primary or secondary human fibroblasts. Transfected cells are plated onto 150 mm dishes in human fibroblast nutrient medium. 48 hours later the cells are plated into 24 well dishes at a density of 10,000 cells/cm² (approximately 20,000 cells per well) so that, if targeting occurs at a rate of 1 event per 10⁶ clonable cells then about 50 wells would need to be assayed to isolate a single expressing colony. Cells in which the transfecting DNA has targeted to the homologous region upstream of hCAR will express hCAR under the control of the mMT promoter. After 10 days, whole well supernatants are assayed for hCAR expression. Clones from wells displaying hCAR synthesis are isolated using known methods, typically by assaying fractions of the heterogenous populations of cells separated into individual wells or plates, assaying fractions of these positive wells, and repeating as needed, ultimately isolating the targeted colony by screening 96-well microtiter plates seeded at one cell per well. DNA from entire plate lysates can also be analyzed by PCR for amplification of a fragment using primers specific for the targeting sequences. Positive plates are trypsinized and replated at successively lower dilutions, and the DNA preparation and PCR steps repeated as needed to isolate targeted cells.

Targeting to Sequences Flanking the Human hCAR Gene and Isolation of Targeted Primary, Secondary and Immortalized Human Fibroblasts by a Positive or a Combined Positive/Negative Selection System

Construction of 5′ hCAR-mMT targeting sequences and derivatives of such with additional upstream sequences can include the additional step of inserting the neo gene adjacent to the mMT promoter. In addition, a negative selection marker, for example, gpt (from PMSG (Pharmacia) or another suitable source), can be inserted. In the former case, G418^{r} colonies are isolated and screened by PCR amplification or restriction enzyme and Southern hybridization analysis of DNA prepared from pools of colonies to identify targeted colonies. In the latter case, G418^{r} colonies are placed in medium containing 6-thioxanthine to select against the integration of the gpt gene (Besnard, C. et al., Mol. Cell. Biol. 7:4139-4141 (1987)). In addition, the HSV-TK gene can be placed on the opposite side of the insert to gpt, allowing selection for neo and against both gpt and TK by growing cells in human fibroblast nutrient medium containing 400 μg/ml G418, 100 μM 6-thioxanthine, and 25 μg/ml gancyclovir. The double negative selection should provide a nearly absolute selection for true targeted events and Southern blot analysis provides an ultimate confirmation.

The targeting schemes herein described can also be used to activate hCAR expression in immortalized human cells (for example, HT1080 fibroblasts, HeLa cells, MCF-7 breast cancer cells, K-562 leukemia cells, KB carcinoma cells or 2780AD ovarian carcinoma cells) for the purposes of producing hCAR for conventional pharmaceutical delivery.

The targeting constructs described and used in this example can be modified to include an amplifiable selectable marker (e.g., ada, dhfr, or CAD) which is useful for selecting cells in which the activated endogenous gene, and the amplifiable selectable marker, are amplified. Such cells, expressing or capable of expressing the endogenous gene encoding a hCAR product can be used to produce proteins for conventional pharmaceutical delivery or for gene therapy.

### Example 15

### hCAR Polymorphisms

Single Nucleotide Polymorphisms (SNPs) found in the Celera human RefSNP database which map in and around the hCAR gene. The SNPs were identified by text querying the Celera human RefSNP database for SNPs lying on chromosome 4 between positions 8316626-8342946; these co-ordinates correspond to the chromosomal location of the 26320 bp contig depicted in FIG. 7.

Reference: the Celera assigned ID for the SNP. #Chrs: The number of chromosomes, related to the number of individuals having the SNP. Variation: The nature of the polymorphism. Frequency: (in %) The occurrence of an allele in the total number of chromosomes. Chromosome: The chromosome on which the SNP is located. Position: The absolute position of the SNP on chromosome 4, according to the Celera Discovery System as of March 2001. 26320 bp Contig position: The position of the SNP in the 26320 bp genomic sequence which includes the hCAR gene (this sequence is depicted in FIG. 7.)

TABLE 3

| | | | | | | 26320 |
|---|---|---|---|---|---|---|
| | | | Fre- | Chro- | | bp |
| Reference | | Varia- | quen- | mo- | | Contig |
| (Human_RefSNP) | #Chrs | tion | cy | some | Position | position |
| CV1221921 | 4 | C/T | 25/75 | Chr4 | 8317095 | 469 |
| CV1221920 | 4 | A/C | 25/75 | Chr4 | 8317194 | 568 |
| CV1221919 | 4 | C/T | 50/50 | Chr4 | 8317491 | 865 |
| CV1221918 | 3 | T/C | 66/33 | Chr4 | 8317672 | 1046 |
| CV1221917 | 7 | C/T | 57/42 | Chr4 | 8318263 | 1637 |
| CV1221916 | 3 | A/G | 33/66 | Chr4 | 8319575 | 2949 |
| CV1221915 | 2 | C/T | 50/50 | Chr4 | 8319961 | 3335 |
| CV7662683 | 3 | C/T | 66/33 | Chr4 | 8321056 | 4430 |
| CV1221914 | 3 | C/T | 66/33 | Chr4 | 8323284 | 6658 |
| CV1221913 | 5 | A/C | 80/20 | Chr4 | 8324706 | 8080 |
| CV1221912 | 5 | C/T | 20/80 | Chr4 | 8324712 | 8086 |
| CV1221911 | 5 | T/C | 80/20 | Chr4 | 8324716 | 8090 |
| CV1221910 | 3 | A/G | 33/66 | Chr4 | 8325253 | 8627 |
| CV1221909 | 3 | G/T | 33/66 | Chr4 | 8326430 | 9804 |
| CV1221908 | 3 | G/T | 66/33 | Chr4 | 8330486 | 13860 |
| CV1221907 | 3 | A/G | 33/66 | Chr4 | 8330515 | 13889 |
| CV1221906 | 2 | A/G | 50/50 | Chr4 | 8331272 | 14646 |
| CV1221905 | 2 | G/C | 50/50 | Chr4 | 8331384 | 14758 |
| CV1221904 | 4 | T/C | 75/25 | Chr4 | 8331760 | 15134 |
| CV7664553 | 3 | —/C | 33/66 | Chr4 | 8331834 | 15208 |
| CV1221902 | 3 | G/A | 66/33 | Chr4 | 8331879 | 15253 |
| CV8280237 | 4 | | | | 8331906 | 15280 |
| CV1221901 | 4 | A/G | 25/75 | Chr4 | 8332905 | 16279 |
| CV1221900 | 4 | C/T | 75/25 | Chr4 | 8333074 | 16448 |
| CV1221899 | 4 | A/G | 75/25 | Chr4 | 8333125 | 16499 |
| CV1221898 | 4 | C/T | 75/25 | Chr4 | 8333160 | 16534 |
| CV1221897 | 5 | A/G | 80/20 | Chr4 | 8336146 | 19520 |
| CV1221896 | 6 | G/C | 83/16 | Chr4 | 8336273 | 19647 |
| CV1221895 | 5 | C/T | 40/60 | Chr4 | 8336625 | 19999 |
| CV1221894 | 3 | G/A | 33/66 | Chr4 | 8336781 | 20155 |
| CV1221893 | 4 | C/T | 75/25 | Chr4 | 8337433 | 20807 |
| CV8280238 | 4 | | | | 8338074 | 21448 |
| CV1221892 | 3 | A/G | 33/66 | Chr4 | 8339805 | 23179 |
| CV1221891 | 4 | C/T | 75/25 | Chr4 | 8339850 | 23224 |
| CV1221890 | 4 | G/A | 50/50 | Chr4 | 8339877 | 23251 |
| CV7664594 | 5 | —/A | 20/80 | Chr4 | 8340292 | 23666 |
| CV7664595 | 5 | G/C | 20/80 | Chr4 | 8340297 | 23671 |
| CV1221887 | 6 | A/C | 33/66 | Chr4 | 8340396 | 23770 |
| CV7664734 | 5 | —/T | 40/60 | Chr4 | 8340421 | 23795 |
| CV1221885 | 4 | A/G | 25/75 | Chr4 | 8340663 | 24037 |
| CV1221884 | 6 | T/C | 66/33 | Chr4 | 8341057 | 24431 |
| CV1221883 | 5 | C/G | 20/80 | Chr4 | 8341182 | 24556 |

### Example 16

### Structure of the hCAR Protein

The peaks of hydrophobicity were determined by the program Toppred and are shown in FIG. 8. The actual locations of transmembrane regions were confirmed using another prediction program (TMpred—Hofmann, K. & W. Stoffel (1993) TMbase—A database of membrane spanning protein segments Biol. Chem. Hoppe-Seyler 347,166) and through the use of careful visual analysis of the amino acid sequence for conserved residues and other indicators of transmembrane regions as known in the art. In addition the GCG program SPScan was utilized to determine the existence of a signal sequence. While this program predicted a signal peptide, more detailed inspection of the sequence for TMs suggests that the predicted signal sequence actually corresponds to an initial transmembrane region, confirming a priori expectations with respect to GPCRs.

The analysis revealed the following transmembrane region locations:

TM1 at amino acid positions 6-29;

TM2 at amino acid positions 42-68;

TM3 at amino acid positions 81-102;

TM4 at amino acid positions 122-149;

TM5 at amino acid positions 174-193;

TM6 at amino acid positions 243-260;

TM7 at amino acid positions 275-300.

The extracellular regions are:

N-term at amino acid positions 1-5 comprising the amino acid sequence: Met Gly Pro Gly Glu (SEQ ID NO: 4);

EC1 at amino acid positions 69-80 comprising the amino acid sequence: Arg Gly Arg Thr Pro Ser Ala Pro Gly Ala Cys Gln (SEQ ID NO: 5);

EC2 at amino acid positions 150-173 comprising the amino acid sequence:

(SEQ ID NO: 6)

Ser Ser Ala Phe Ala Ser Cys Ser Leu Arg Leu Pro
Pro Glu Pro Glu Arg Pro Arg Phe Ala Ala Phe Thr;
and

EC3 at amino acid positions 261-274 comprising the amino acid sequence:

(SEQ ID NO: 7)

Arg Leu Ala Glu Leu Val Pro Phe Val Thr Val Asn
Ala Gln.

Those skilled in the art will recognize, or be able to ascertain using no more than routine experimentation, many equivalents to the specific embodiments of the invention described herein. Such equivalents are intended to be encompassed by the following claims.

1. An isolated nucleic acid molecule comprising a nucleic acid which encodes a protein comprising the amino acid sequence of SEQ ID NO:2.

2. The nucleic acid molecule of claim 1 further comprising vector nucleic acid sequences.

3. The nucleic acid molecule of claim 1 further comprising nucleic acid sequences encoding a heterologous protein or a fragment of a heterologous protein.

4-12. (canceled)

13. An isolated hCAR protein comprising the amino acid sequence of SEQ ID NO: 2.

14. (canceled)

15. (canceled)

16. The protein of claim 13 further comprising heterologous amino acid sequences.

17. An antibody which selectively binds to a protein of claim 13.

18-20. (canceled)

21. A method for detecting the presence of a protein selected from the group consisting of: a) a protein comprising the amino acid sequence of SEQ ID NO:2; b) a fragment of a protein comprising the amino acid sequence of SEQ ID NO:2, wherein the fragment comprises at least 15 contiguous amino acids of a transmembrane or extracellular region of SEQ ID NO: 2; and c) a naturally occurring allelic variant of a protein comprising the amino acid sequence of SEQ ID NO: 2, wherein the protein is encoded by a nucleic acid molecule which hybridizes to a nucleic acid molecule comprising SEQ ID NO: 1 under stringent conditions; the method comprising the steps of: i) contacting the sample with a compound which selectively binds to the protein; and ii) determining whether the compound binds to the protein in the sample.

22. The method of claim 21, wherein the compound which binds to the protein is an antibody.

23. (canceled)

24. A method for detecting the presence of a nucleic acid molecule selected from the group consisting of:
a) a nucleic acid molecule which encodes a protein comprising the amino acid sequence of SEQ ID NO: 2;
b) a nucleic acid molecule which encodes a fragment of a protein comprising the amino acid of SEQ ID NO:2, wherein the fragment comprises at least 15 contiguous amino acids of a transmembrane or extracellular region of SEQ ID NO:2; and
c) a nucleic acid molecule which encodes a naturally occurring allelic variant of a protein comprising the amino acid sequence of SEQ ID NO: 2, wherein the nucleic acid molecule hybridizes to a nucleic acid molecule comprising SEQ ID NO: 1 under stringent conditions; in a sample, the method comprising the steps of:
i) contacting the sample with a nucleic acid probe or primer which selectively hybridizes to the nucleic acid molecule; and
ii) determining whether the nucleic acid probe or primer binds to a nucleic acid molecule in the sample.

25. The method of claim 24, wherein the sample comprises mRNA molecules and is contacted with a nucleic acid probe.

26. (canceled)

27. A method for identifying a compound which binds to a protein selected from the group consisting of:
a) a protein comprising the amino acid sequence of SEQ ID NO:2;
b) a fragment of a protein comprising the amino acid sequence of SEQ ID NO:2, wherein the fragment comprises at least 15 contiguous amino acids of a transmembrane or extracellular region of SEQ ID NO: 2; and
c) a naturally occurring allelic variant of a protein comprising the amino acid sequence of SEQ ID NO: 2, wherein the protein is encoded by a nucleic acid molecule which hybridizes to a nucleic acid molecule comprising SEQ ID NO: 1 under stringent conditions; the method comprising the steps of:
i) contacting the protein, or a cell expressing the protein with a test compound; and
ii) determining whether the protein binds to the test compound.

28. The method of claim 27, wherein the binding of the test compound to the protein is detected by a method selected from the group consisting of: a) detection of binding by direct detecting of test compound/protein binding; b) detection of binding using a competition binding assay; and c) detection of binding using an assay for hCAR activity.

29. A method for modulating the activity of a protein selected from the group consisting of:
a) a protein comprising the amino acid sequence of SEQ ID NO:2; and
b) a naturally occurring allelic variant of a protein comprising the amino acid sequence of SEQ ID NO: 2, wherein the protein is encoded by a nucleic acid molecule which hybridizes to a nucleic acid molecule comprising SEQ ID NO: 1 under stringent conditions, the method comprising the step of contacting a cell expressing the protein with a compound which binds to the protein in a sufficient concentration to modulate the activity of the protein.

30. (canceled)

31. A transgenic or chimeric nonhuman animal comprising the nucleic acid of SEQ ID NO: 1.

32. The animal of claim 31 wherein the transgene is under the control of a regulatable expression system.

33. A knockout nonhuman animal wherein at least one allele of the HCAR gene has been functionally disrupted.

34. The knockout nonhuman animal of claim 33 wherein at least one allele of the HCAR gene can be functionally disrupted by the induction of the Cre gene.

35. A knockout according to claim 34 wherein the Cre gene is under the control of a tissue specific promoter.

36. A knockout according to claim 34 wherein the Cre gene is under the control of a developmentally specific promoter.

37-38. (canceled)

39. A method of inhibiting expression of the HCAR gene in a cell comprising providing said cell with an antisense nucleic acid.

40-41. (canceled)


SEQUENCE LISTING (7 sequences)

SEQ ID NO: 1 - DNA, 5665 bp, Homo sapiens
  5′UTR (1)..(1891)
cccactcttcctggacatgtcaggaaaactttgacgtggctgctctagcttcagggaaggtctaatttggtgaaaatttgaaagcaggtttgtgggagtgccagggagaaatggggagagagaaagcctctgtatttgatggatggcaatggcttggagctggtgtgatggcctctctggatgacaaggacattggacttagagccagaaggactgaggtatgaatctcggcattcctggtttgtagttatggggacttggcagagacacttgaatgaaacttcctttgcccaggtataagacggaccccctaataaaggttgactgtgttctgatccttcactgcctgctgggatgtcctcagcattttgtgcatgttggccaatttaaccctaacagcaaccaccagaggcagatgctattgctggctattaatatccccatgtgacagatgagaatgtgaggcccaaggggtttaagtggggctatgaatatccccatgtgacagatgagaatgtgaggcccaaggcacagaaccaggggtgccccagcatgccagttgtgcacctgtggcttttcccttggccactttgcagcaccggcacgagagaggcccacagggtgagcctccacaccaccagccaccctttgtccctcagaaagggctggcagagcctgcaggtgagggtgggtgtggggaggggtgggcaatcgtctgcccttcatttctgtcatgttgtggctgtcactggggagaaaatgccaaaaagcttcctggaagaagcagcttccaggaggcttcaccatatccttgtcctgccaagtggccacgaatggattagaagattcccactgggtgagaaggctcagaagccaccacagaggatggcagaggtgggagaggcctcgcaccgcggggctccaggagccaggtgaaggacaggcatttctgtatggcacccagttctgggtgggtcctcccaaggtgcccccttctttgtctctccctctgttgcttttctctcctcttccctcttcttccccccacttcctcttcactttttcttcactttttcttcttcttctctttccttcccccatgcctttctcaaccttgtttccacttcttgtcgctcttcttgcttcaacaaacgtcgatgcagtcacagttcctgggctgaggctgggggatgggaggaagtcctgagggcagcccccgcccccttccccgccccgtcactccctctgccccgcctgcacagcttcttgccaattcattcccgcccctaccgcccctataagccaccaggtcgctccagtttggtgccagcgcctggagggagaggcgtggcgagggctgtgctgcctaggatccactgagtggctcttgctggcgtgtcagctgcgcgcgaaccagggctgggaggctcggctggaggtgtgaccagggcagggactgacctggcccggaacagaagcgcgcagagtcccatcctgccacgccacgaggagagaagaaggaaagatacagtgttaggaaagagacctccctcgcccctacgccccgcgcccctgcgcctcgcttccagcctcaggacagtcctgccgggacggtgagcgcattcagcaccctggacagcaccgcggttgcgctgcctccagggcggccccgggctgctcctgctccgcagagctacgccctccccccgggtgccccggaccctgcacttgccgccgctttcctcgcgctgctctggaccttgctagccggctctgcacctcccagaagccttgggcgcgccgctcagctgctccatcgcctcactttcccaggctcgcgcccgaagcagagccatgagaaccccagggtgcctggcgagccgctagcgccatgggccccggcgaggcgctgctggcgggtctcctggtgatggtactggccgtggcgctgctatccaacgcactggtgctgctttgttgcgcctacagcgctgagctccgcactcgagcctcaggcgtcctcctggtgaatctgtctctgggccacctgctgctggcggcgctggacatgcccttcacgctgctcggtgtgatgcgcgggcggacaccgtcggcgcccggcgcatgccaagtcattggcttcctggacaccttcctggcgtccaacgcggcgctgagcgtggcggcgctgagcgcagaccagtggctggcagtgggcttcccactgcgctacgccggacgcctgcgaccgcgctatgccggcctgctgctgggctgtgcctggggacagtcgctggccttctcaggcgctgcacttggctgctcgtggcttggctacagcagcgccttcgcgtcctgttcgctgcgcctgccgcccgagcctgagcgtccgcgcttcgcagccttcaccgccacgctccatgccgtgggcttcgtgctgccgctggcggtgctctgcctcacctcgctccaggtgcaccgggtggcacgcagccactgccagcgcatggacaccgtcaccatgaaggcgctcgcgctgctcgccgacctgcaccccagtgtgcggcagcgctgcctcatccagcagaagcggcgccgccaccgcgccaccaggaagattggcattgctattgcgaccttcctcatctgctttgccccgtatgtcatgaccaggctggcggagctcgtgcccttcgtcaccgtgaacgcccagtggggcatcctcagcaagtgcctgacctacagcaaggcggtggccgacccgttcacgtactctctgctccgccggccgttccgccaagtcctggccggcatggtgcaccggctgctgaagagaaccccgcgcccagcatccacccatgacagctctctggatgtggccggcatggtgcaccagctgctgaagagaaccccgcgcccagcgtccacccacaacggctctgtggacacagagaatgattcctgcctgcagcagacacactgagggcctggcagggctcatcgcccccaccttctaagaagccctgtggaaagggcactggccctgccacagagatgccactggggacccccagacaccagtggcttgactttgagctaaggctgaagtacaggaggaggaggaggagagggccggatgtgggtgtggacagcagtagtggcggaggagagctcggggctgggctgcctggctgctgggtggccccgggacagtggcttttcctctctgaaccttagcttcctcacccttgttctggggtcatggcgatgcttcgagacagtgggtagggaagtgccctgtgtggcatatggtactcgtgggcgtgctataagtgactgctgttcatgtgggtgaggtggtcactcttgctcagggtctgttgtgcagcccagatggacacctgtttctccaacctggttattagcattgttccgatttgttctcggcattgcccaggtttgggagataaatgccggggcggagtctggttgggggctcccagagttcacatctgatagtctgtggtcaggacctggcaggcacgggcagtccctgggacatgcccatctctggaagcctaggggtccccagctccaggcctgtccgctgtgactgcctgtgtgggcacgcagatggagcctgtctcctgccttcctttccatggtttgccaggggtttggcatcttgactgcggaagctgtggagtctgtgtgctcagagccttttctggtgaagatatcatcagagcatgtgacctctgtttcctccccctgaaggccaccgctgggcctctggatcttagacatgagacggtcaagagattgaagtagtagccagggcccaggtgtccagagagggtggcctgggatggggagggcccttgctccccaacagcagtgctgggggagccaagagaaggtggagcatccctgagtagtggtgtgcatcacccccagtttagtaatcacggggtgccattccccggtgggagcacccaccatcaatgtcattgaatgtccccatgggacagtgttgaggacttttgtgacatctgtcctatttcacagctcagggaaaggtgcacagtgcacacgggcacccggtggagaggtgtgtgtgtgaatgagtgagcgagtgaatgaatggacacgattctctcttcagcctctgtcattgctgttttcttcaaggcccagggccatcccctgcagaggcagggtgggctgcaagacctcaggccctgcctcatggactctctgatgggcttcaaccgtgggctctgcaggcatggagcctgtatcatgacaccttacacccaaggccagcaatgcaaggagagtatggacatcaaattctttccttccagaggctgaattcttcaaagacacacgcggtcgtcccttgctcttggcattaacggtggagaacccagctgaggtggcttcacagattcttccccaaaaacacaggtgttattattatacttttaaaaaactttttgagacagggtctgactctgttgcctaggctagagtgcagtggtgcaatctcagctcactgcagcctccacctcccatgctcaagccatcctcccacctcagcctcctgagtagttgaggacacaggcacgggacaccatgcctggctaatttttgtatttttttttgtagagatggcggtctcactttgttgcccaggctggtcttgaattcctgagcttaagtgatcctcccacctcagcctcccaaagtgctgggattacaggtgtgagccaccacacccagccaaaaccaggtgttatttgctgactcaccaatgcctcccccaaaaggataaatttaaaggtgtgtataactcatgaagtgtaattcaaataaaacaaattatcgtttggaataataacaactataggtatgtggtcaggaagcagttaaaaacattaaaatacagacctggccagttcaatccaacccaggggttccaagcaggaggtggggcaggtgggcgtcatgccctgattcacagagggcacaggtgggtgtcatgccctggttcacagagggcacgtgaactcgagaccgtgctgcaccccggtgcccctgtgcttataagggagggcacgtgcacagcagaagcaggttgttccccatttaaagttctggagcccaggctgtgagctccttggctgagccctctcctgtccctgggagctccccaggtgcgaggagcctgccagccagtggggcctacactctgtgttattgcatctccgccaggctaaaagccttggtcactactttagagccactcaaggaaacgcgtgcaccctgccctgctggaaggcaccatggttagagggaggcacactgtttcttagagacggggactgcttgctgtcatgtttcgccttcctcggaagctccatggaatgttctggagcaggcatcttagggcattccctccgcacttctctgccagcccatgtggctcccacactgggctatcccttgccttaggcttgtggccttttttttttttttttttaatttgaaaaatatttttcatgtgcacttaaacgtgttgtggaatgatgctgggtctcaagaatgctgtgaatcaataaacattttattcagaaaaaaaaaaaaaaaaaaaggcggccgc

SEQ ID NO: 2 - protein, 363 aa, Homo sapiens
MGPGEALLAGLLVMVLAVALLSNALVLLCCAYSAELRTRASGVLLVNLSLGHLLLAALDMPFTLLGVMRGRTPSAPGACQVIGFLDTFLASNAALSVAALSADQWLAVGFPLRYAGRLRPRYAGLLLGCAWGQSLAFSGAALGCSWLGYSSAFASCSLRLPPEPERPRFAAFTATLHAVGFVLPLAVLCLTSLQVHRVARSHCQRMDTVTMKALALLADLHPSVRQRCLIQQKRRRHRATRKIGIAIATFLICFAPYVMTRLAELVPFVTVNAQWGILSKCLTYSKAVADPFTYSLLRRPFRQVLAGMVHRLLKRTPRPASTHDSSLDVAGMVHQLLKRTPRPASTHNGSVDTENDSCLQQTH

SEQ ID NO: 3 - DNA, 26320 bp, homo sapiens
  gene (1)..(26320)
tcatggcagatttaaagcaggcagaaaagaaaatagagcagtaggcagagaacttaggaaacctatagtcgcaggtccaactttgtgctctgaatttttcttgatgaaatttgcctatcagtttaaaatctgcacaagaacagaccatcatatgtaaccagctggagtactagaaaacctggcatgcttttgactttcccattttttaaaccttaattatcctcatattttcttaggatctgaaagaaagctgcaacaacattaaaaaaaattatcttttgtagagacagggtttcaccatgttgcccaggctggtcttgaactcatgacctcaagtgatccaaacgccttggcctcccaaagtgctgggataacaggtgtgatccaccacacccagctctacaacaacaaatttgagagaacttcttaaattgttgtttgattctgcaggagtaatgtcacttggggtgcccacatagaagggacccccttaaccacagcatttaccatgacctgggtaatgggtatattctatgggtgaatatcctggtcatcataaagccagtcccacatggcttgtatatgaagcatatcagttgcttcatctggggtgctccacttggcatttataggaagagttgggcagtccccttctcagggtaaacagactccatggtggctttcatctagtcagacaggctggccgttgcctcaggcataacctcctgtgcagctggatcacatatactgatcagggagtgttcaatagtgagctgtgggtcctgcatcaacccaaacgtgccctttcattctgtagcatttaaaattaaagatactgtccatgaagtagttactctcaccacctattttagtaaaagtttcacaggaagctgatgataccaatctgtaaaatggaacaattccttgacaccacatcctctgggtttcatagttagttggtttttccctttctccacatggactgtcttcttggtacccacaggtctcagaggtactttctgctgccctggcttaatttttccttctgtggatagctttgaggctggtgatctgagcccagacagaccatatctgagtttggtccagcctcaaggctccccaccccaggcccaacatttcatttgacttttagtttagctattacagataacagtaaccaaggggctgagtatgttgctttatgcatccagtgaaccaactcaacagtgtgccccatctccaaattctactggtgcctttgaccctcagtgactgaggcagcccagctgcagctccataccatgggacacttagtaagcacctgctgtatgcttagcctgggctgcttccaacttgttttatctcttcctgccacccccgacaatctaagcagatggctcctgctgtcttgcagaagaggaaactgaggtgcagagagatgaagtgacttgtccaaggtcatacagtgacccagtgtctgagccagggtcgggaccgctttgccagatgctggctacagaagctggtgctctgcctcccataggcacccctgagtcaccagccacggtgcagagtgtaattaggcctgactcatgcagtcattatactccaggttttaattatccgcctcctcatcctggtgtcctctttgggccagttatggaggccatgggacaggccttctggctggggtgtctccttgtatctgtcaggcaaagaattgaaaaactgttggggacatgcccaacagtggctgaaacagaaataatgggtgctgtgcagagtgacagggagagcttgagttctctgcaggcaacttagctgcaattcagcaactggaattcccagtttggccaccagcatggagggtgggcagggcccgcctaatgactgcttggattccagctggtgccagagggcagggtccagggcttggagtccaacagtgggggtggttcaggctcctttcagctctgtggccccaggccagccccctggtctctctgggggcaagtttctccagtggcaaattgagaagaaaaaaaaacctgcctcattgtttgggtgcttatcagatggggttaagcaggtgaaatgtccaatgcactctaagagctgttgacaatgtttgccttgtgtcttcatccagtcattgcatataagccgtggtgagtagatatggctcacgggaactgggcaggtggaatccacacctcaccgcgttgctgggaagatggaggaggtgacgcatgggaagacctccccagtaagtgttggctgctcccagtatcacgagaatgattcctggtggaggtcataactgtgttaccattacctgcgatgtctggaaacatttttgccaaaggtggcactgcctgtgcccaggctatgtgtcctgtccagaccaggcgtgtggtgggctccttctaggctcctcagaggtgcatttcacatgtttatgttgtgtgacaaacgctggttttatccatcttgtcctcagacctacaaccagcatttcctaaatgaacagttggactgtttatttaaaatgttattcccatgaaggctaaactcagattgaagccttcctgggaagcttcacacatcctctccccaacctctttcttcacattttcaaccaagtttcttttctttctttttgagccacagtctcgctctgtctcccaggctggagtgcagaggtacaatcatagctcactgcagccttcaactcttggactcaagcgatcctcctgccttccagagtagctgggaccacaggtgtgtgccacctaggtccaccatccagcttcttccctggaaccatgacgcaggtggtttctgctctgacagggcctcccatgtcctaggtggaggggccgcaccttgcggggacccacagcctcagcctcaagcaagcagtgggcccttgtggccatggaggccccactgtgtctcttgattcctacttaaagatacaccatggtttcccttaccttccacctagtgagaatcctaactgttctcatggtaaatgtcacacttactgtgcctaggcgtccatgagagacaggagaagtttcttcccacactaaagataataagatggcggaaagatggtcaccttccctgcctgaggccactcacatccaggaggcagcgggggaggggaagaggggaaggatttgaacccaagggagagcctgtctcttaggccgttctgactgcccagtgagaactgatggcctctcctctgtggcctgctgcgccttacctgcacccctctctccaggcccctgttccaccctgggtgtggaggcagccatcaacctcctggttcctggctcagccctggcgcataagaggggcaagcgcttgtggaataagtgggtgaaaggatgcccatgggtctcctttgtcctggctgcagcccctctgtgagtgcaccttgggtggcatcgtctgagcatcggcgtttccgggtgaccgctgtgggggcggttgtgacactcgtggtgacacttatgcctcttttatttattaaaaaaatgttttttggggccaggtgcagtggcttgtgcctgtaatcccagcactttgggaggccgaggcgggtggatcacctgaggtcaggagttcgagactagcctgaccaacatggtgaaaccccgtctctactaaaagtacaaaaattagccaggcgtggtggtacacggcgtgtagagtgccactacactctagcctgggtgaaagagactctgtctcaaaaaaaaaaaaaattttttttttttgagacagggtcttgctcagtcgcacaggctggagtgtagtggtgtgatcttggcttactgcggcctctatctcctgggctcaagtgatcctcccacctcactcccctgagtagctgggattacagctgtacaccaccatgcctggctaatttttacatgattttctggagatggagtttcatcatgttgcccaggctggtctcaaattgctgggctcaagtgatccgcctgcctcagcctcccaaagtgctgggattacaggcatgagccactgtgcccagcaactcatgcctcttttaatcctcataccaaccctatggggaagcttctgagttcccacgctgcagatgaggaaactgaggctcagggaggatgaggccatactgctcagaagagaaggtgcagaatcaacccaggcctgaatggctcctcagccggaacccttttccttcctagtgccagagttttcttccaagtatcggggagacactcttaacttggctgtggattctttccatcctgatgtcctccatctggttgaggctagggcctacctcctcagcctcctgttgcaggaggcctgccagggtgggccacccctggggccagagcagccaaggggcccggttggctccctgcactggggctgcctctgggaacagctttccagagttgcaggtgcttcaggaggacaggaggccaggtgagtggcccagcatgatgccctggctgcagggttgtctctgaggataaagggaccctgtgcaggtgatcaggtaggcagggctccgggctgcttcgtgaccttcagctgagactctaggaccagcccacacaaacccctttctcctctggagggttcttcacacagggtggggccagtgcagagctggtccttcccagctgagagcttcttagcagcaggagctcaggctgcactgaccaagaccccaaaggcctccagggctgccagactgaaaggtcaggacacagcccctgccagcagcctttgctgacatcccacagcctctaggacaaatcccaaatcacttagcctggcattccaggttttttaggggttgccctggccatagtccacaaccttgaactttgcttcccgctgaccccttcctcctccttaccatctgataatctcttactcatcccttcttgtcaaactctgctgtccttcaaaattctcttgctctggggagacccaggctgggtcaggtgccacccaagggtcccccagagcctggagcctccctcagcctcagtcatacctggaggagtcatgcccagtttctggcctgcccttggcccccggcccgtgaactcccagaagacagggacagatcttatttgccctgtagtcacaggtcccagctccatggcactgagggacccttccgtgtttcattgaggaattagtgggaatgtgttgctgctgagggcatgtgtgatttctaagtgtgtggataatattgccagctgtaatactttcctacctctctgctattctaaggaattccgcggatcctctccctatggtcagnnnnnnnnnnnnnnnnnnnnnnnnnnnnnnnnnnnnnnnnnnnnnnnnnnacaaccaaccactcctagctccatcagccattggtgaccatgactggtggcctggcatcaggatccggtgcttctataccttgttcagaggtcccagtgttctcagtggtttccgctaaacaatccacagtgggatttttccttgggacatctgcttttctttctctctcgctctttctccctccctccctccctccctctctccctctctcctttttgtcttgtcttttctcttttcttttctttcttagatggaatctcgctctgttgcccaggctggagtacagtggcacgatctcagctcactgcaaactccatctcctggattcaagtgattctcctgtcagcctcctgagtggctgggattacaggtgcacaccaccatgcctggctaatttttgtattttatttatttatttatttatttatttatttttagtacagatggggtttcactatattggtcaggctggtctcaaactcctgatccacatgccttgggctcccaaagtgctgggattccaggcatgagccaccatgctgggctgacacctgctttttaatggccgcacaggactatgctgtaggggaatagaatcacgcacttgccgccttcccgtttttggaagtttcattcatttccctgtctttccaccttcctcctttctcttttccgcctgtctatgagcaatcatttttgttaacatgccagattgttccttccagttgctttcctgcagtcagggcttgatagctgtgggctcccccagcccctccaaggcttctaagtgaccatgtgcgtgcagctctggggagtggtggaggtgtcaggaggtttcacaaggggactttggtgtagccactcacagcggcccctgtcacctctcaatgactccagggggagacgatttcacttgcaggggagcagagagcatctcttctgagacatgagcgcagggaagcttcgccagcctgtattacggaaaggcaggccattcactcatctgcaaggatgtactgggcacccagcgtgtacaagagaatgtgctggatacagcagactgtggtgatcacacaaggcttgatttcttcactttgttgagcagagagaagacgtggacaaagtcagatctgatcaatacaaccaccctggccccccaggagaaacagcaagcttattttgtgagataagtgaattgtatcaattacatcatattgcagtgctatcactgatgacaaattcatttccacagatatacaccgacagcttgttacatgcaactggggaggaaaaatgagaaaagcgggaaaagaacaatggttaactgagctcatttgattttatttaaacttgctttagtactaagcttatttcttttacacctattgaatgtaaccttatttaattcttaccccacccctcgaagatgggtcttcctgcccctatttttcagataaggaaacaattaagccattcactttaattaagcagcagtggcatctcagcctctgcacttcatctcagcccagcacttctgttggggctgagatggaagtctcggaaggtgctctgaggaggtgtgactctccctggctgacaggggaaggcttagcagagctttgtcttagaggagtagatgaaaaggaaagtacagagagggcattcaggccaagtcagcaacacagacaaagtcaggtaatgtgggttaagtgcatgggtgatgagtaaaggggatgtggctagatggtgtgagtgtgtgtgtgcttgcatgtgtgcctgtgtgcgtgtgtgtgcatgtgtgtgtctgtgtgtgagtgacagcaacagcaaagggcccgtcatgagtggctaagaccagatgtaggtagacttggaggggctgctaaggaatttcataggcaatggggaaccatgaactatcactaggcaggggaggagccttcgggaactaatcctgaactttatcattgcaatgcgtatctcagcaagaatgggcaggattgcatagttaggagtactgccttactttttaaagaagtggtgaaaatatttaatattttcatatactttttctttttggaagataaaaggattaggtccagcatttcaccctacagaggatttaaatttttcatcaggaatgagatttgagtgtaagaagatgaaacgatattatactgataagaccacagggttcaaaaccaccccctacaacccagggaaggggggcagccaggctggcaagatctgaggccagaagcactggggctttggggagagcagcaaacagaacagatctagacctatcaaggtgccctcaccagagtccagagatctcaccacacacattttctcattcatgcagttgtccagtacatccttgcagatgagcaaatggcctgcctttcataatacaggctggtggagcttccccatgctcatgtctcagaagagatgctctctgctcccctgcaagtggaattgtctccctttggagtagttgagaggtgacagaggctgctgtgagtggctagaccaaagtctccctgtgaagccacgtgatgactccaccgctccccagagctgcacatggtcgctcagaagccttggaggggttgggggaggcccctgctctcaagcccccactatcacaatcactttgtcccagctattcacctgcaaacttgcttgatctgcagaagctgcagagtggcccactcttcctggacatgtcaggaaaactttgacgtggctgctctagcttcagggaaggtctaatttggtgaaaatttgaaagcaggtttgtgggagtgccagggagaaatggggagagagaaagcctctgtatttgatggatggcaatggcttggagctggtgtgatggcctctctggatgacaaggacattggacttagagccagaaggactgaggtatgaatctcggcattcctggtttgtagttatggggacttggcagagacacttgaatgaaacttcctttgcccaggtataagacggaccccctaataaaggttgactgtgttctgatccttcactgcctgctgggatgtcctcagcattttgtgcatgttggccaatttaaccctaacagcaaccaccagaggcagatgctattgctggctattaatatccccatgtgacagatgagaatgtgaggcccaaggggtttaagtggggctatgaatatccccatgtgacagatgagaatatgaggcccaaggcacagaaccaggggtgccccagcatgccagttgtgcacctgtggcttttcccttggccactttgcagcaccggcacgagagaggcccacagggtgagcctccacaccaccagccaccctttgtccctcagaaagggctggcagagcctgcaggtgagggtgggtgtggggaggggtgggcaatcgtctgcccttcatttctgtcatgttgtggctgtcactggggagaaaatgccaaaaagcttcctggaagaagcagcttccaggaggcttcaccatatccttgtcctgccaagtggccacgaatggattagaagattcccactgggtgagaaggctcagaagccaccacagaggatggcagaggtgggagaggcctcgcaccgcggggctccaggagccaggtgaaggacaggcatttctgtatggcacccagttctgggtgggtcctcccaaggtgcccccttctttgtctctccctctgttgcttttctctcctcttccctcttcttccccccacttcctcttcactttttcttcactttttcttcttcttctctttccttcccccatgcctttctcaaccttgtttccacttcttgtcgctcttcttgcttcaacaaacgtcgatgcagtcacagttcctgggctgaggctgggggatgggaggaagtcctgagggcagcccccgcccccttccccgccccgtcactccctctgccccgcctgcacagcttcttgccaattcattcccgcccctaccgcccctataagccaccaggtcgctccagtttggtgccagcgcctggagggagaggcgtggcgagggctgtgctgcctaggatccactgagtggctcttgctggcgtgtcagctgcgcgcgaaccagggctgggaggctcggctggaggtgtgaccagggcagggactgacctggcccggaacagaagcgcgcagagtcccatcctgccacgccacgaggagagaagaaggaaagatacagtgttaggaaagagacctccctcgcccctacgccccgcgcccctgcgcctcgcttcagcctcaggacagtcctgccgggacggtgagcgcattcagcaccctggacagcaccgcggttgcgctgcctccagggcggccccgggctgctcctgctccgcagagctacgccctccccccgggtgccccggaccctgcacttgccgccgctttcctcgcgctgctctggaccttgctagccggctctgcacctcccagaagccgtgggcgcgccgctcagctgctccatcgcctcactttcccaggctcgcgcccgaagcagagccatgagaaccccagggtgcctggcgagccgctagcgccatgggccccggcgaggcgctgctggcgggtctcctggtgatggtactggccgtggcgctgctatccaacgcactggtgctgctttgttgcgcctacagcgctgagctccgcactcgagcctcaggcgtcctcctggtgaatctgtctctgggccacctgctgctggcggcgctggacatgcccttcacgctgctcggtgtgatgcgcgggcggacaccgtcggcgcccggcgcatgccaagtcattggcttcctggacaccttcctggcgtccaacgcggcgctgagcgtggcggcgctgagcgcagaccagtggctggcagtgggcttcccactgcgctacgccggacgcctgcgaccgcgctatgccggcctgctgctgggctgtgcctggggacagtcgctggccttctcaggcgctgcacttggctgctcgtggcttggctacagcagcgccttcgcgtcctgttcgctgcgcctgccgcccgagcctgagcgtccgcgcttcgcagccttcaccgccacgctccatgccgtgggcttcgtgctgccgctggcggtgctctgcctcacctcgctccaggtgcaccgggtggcacgcagacactgccagcgcatggacaccgtcaccatgaaggcgctcgcgctgctcgccgacctgcaccccaggtattggcccagtgcatgccgacaggcccaggccagggacttgggcgctccctgggcagttggcttgaggagcctgtgggcatcaccaccgttactccgcccagagttcaccagccacagcactgcccctgcacgctgctcacaggggtttcctgttggttcattggtgcagacactgcgggggcctctgcctcctgggatatgtgctcagtgcacagggagctttgcgcagagctgtggggtgtgcttctccgggaggggttccgcgggctctgctgtgggcggccagacacacccctcctgtgcatggctgtgggtctgaggcatctgcttgtttctgcccactgctgacccagtgcccttgcatggacttgggcttcaagtcttgagcagggtccatcccccatgctttctgctcactacttggcaggatttgtctcctgaatctctagctggtgtcgccattgttccaaattacctcctgtaggtgttcccctttcccccttggccccccacagaacacccaagaggctctcctgctcctggggtgggcaggccacgtgacttctccctccttgagggccccagccttgcccctccatcagcagcctgcctcagttgcctctgtgtggagaaggatgagccagagggaagggaggaggagctggcccttctttccagacttcctttatttcacctacaaaaaagcaagcctaccccttgttctttctctctcccccatccctctgtcctcctcaggttccaggctaaacatcctcctgaaaccctcctgtctgccctcacttcagcccctgggctctgggcctgggttctgtccccaccctgccatcatcctgaccactgtcctctgtccccacagtgtgcggcagcgctgcctcatccagcagaagcggcgccgccaccgcgccaccaggaagattggcattgctattgcgaccttcctcatctgctttgccccgtatgtcatgaccaggtgggtcctggcagtccggctcctgttgtgggaacagctgggtgggcttggcctcagttgagtaggcctctgaggtttcccagcaagatatctggagggcggccaccaccagaggaccctcctccacacctgacgggctcagggctgtgcttcagctcctgggaaagatcctgggagggaggtggcactggctcccatcctgtcctataaatgaggagactctccttgtccaggcacaggcagatatggggtctgtgaatcagcacctggctctttaaacctagaaagctttcaaaatcaggcaacctgggactaactcaggcctcagactccgcatctcctgggcgtggagttgggaatctgggtggaagctccagctggagcctcggggcagtaacactgccaggtgagtgttctctttgcttctctctttcctggagaccttggcctgagtgcttgtcaggtcagaattacctggagtcacaggtaatttgggaaagagtgtgtgtaaagggcctgctggtaccatcatcacagtgctgtgctgaggggcaggggagtctgtagtttttgcctccggggttcctgggtcacccactcggccacttggttactaccttagctccactcaaggaaatgtgtgcaccctgccttgctggaaggcaccgtggttagagggaggcaggttgtttattagagatggtgactgcttgctattacgttttgcctttcttggaatctccatggaatttctggccaggcccctgaagcgctcaggctgtctgggaggtctccaccctatgtgttgagccatagcaggaggataccctgaaggaagacgctcctgggaggggtccatggccctcatcctcaaggggcccaggtccccaccccaggggagggagccagcagggagtatagtaccaggatcctggctctgtttgtgtagggtcctcctgaggttgctatctaatgccctacaaggtctgccagcctgtccaggatgactgcttgtcttcccaggttactgggtggcttagatatgttgttgggtgggtgggggggaaacagttgtgtcccaggaacccgggaggccccagagtgcccacgctgagtggcaccagctctctcctgcctgccaggttccctctgagctcttcagaatcagcacgtgggacaggagctcttggcaccactgatcccatctggcttagggacagggagccaggtttcttgtggatcacaggcatcctccttcctcacataaaacctggcaaaggcctctggttcttaggaaacttcatgggcacaagtgtggatggggaaactgtagcatccttctcccgggaaccagctatttatctgattatcatgtttgtgtgtatgtgtgcccatgtgcctgcgcatgcatgcatgtgtatgttgtgagggttcatgtgtatacacacatgcatgtgtgtattcatacatgcacatgcatgtacatatgtgtagaaggtgagtgctaaaatttcaagagctccagcatttagttttgaaattttaaatgaatgatgagtgagactggcttactcagccatattatgttgattcatgggtgtaactgttattttcctgtgtgtttacaatgccctgaacatgtatgtgagcatgcatagctatgagtgtgcacacgagtgtaagaggaagaatcccaaacaaagtcatgaagcctgcctttgcagtgttgtttccacaatgtcgttaccaactctgggatctgggcagctcaggctctcttctctgtgaaaaatgtgggtacaatatagcactaacgaggttatatatttcttcatgcactcactaagcatttaatgagcacctactctgagctgggctttgtgcctggtcatgcagttacaaaagcaaatggaacacaggctcctctcggttctgatggtgggagatgcaggtcagcaagaccaggcaggtgtttcagtggggggaggggtcttgagggctggcgcctgggagcagcacagctggaccagtttggacggacctgcctcacaggcgcctctcacatcataggagtgaaccgaatcagaacctcactcctgagtgtgtgtggatgaggttctaatgcagttggtggcttaacccacaggaagtcagagatccaggagtgtgttgtccctgctgccccttaaaaggtagtgcactcattccaattgtcctgcaattgctaaaaatatttcagagctcttctcctgggaattgttttcagaactgggctgcattgttagactcttctgtggacatctagaaggtgaatttaatctggttagaaataactccaagtcaccttgagccagttcatgcggataaagtaggtaaaggagatgggggacatgggatttttattttgtttcttggttgtcactcttgagttttactgactcaatcagtcgtgcagatgaagcccgtggggtgtgcggcagtgagaccaggagggcagggatacagagccctcccttgtggagtctgtggtctactggagacgggggagggcagatggctacggtcacaaggtgttgagcagcagtggacacgtgagcgagccctcttctctgcagggtcatggctggtagttgatacatgagttctggtcccattgtggctccagacacagcctcagctgggtgtttctgatgacttccacctgcaccgtggcttaactaggggatagacactctggcatgtgtgtggctttcttactcacctgcagcccagtcccctagtggctgaaagaaatgccagcctcctcagatccttcgataggaccttccttcccacttgcctgatggttggctgtgaccagtctccagtctatgcctgatggtgggctccctgtatgtgggggtgacagtgtacctgatgtgcatgtgctggccggtggcaaagccctttccggactatcacttactgtaactggatcctcaccgcagcctggggtaggtggttagagccatgactgccttggatctgtgtccctgccctgatttttggattctggatgcctctgctctggctgtgcccctcaccttctctctgtcctcacattctggccctgcccctcagcttgaggtggttgcctgcctgcctgcctgccagcctcctcatccccatcccgccggactgggtgcctcggagggagggagtcagtctgatccattgctgcaccccagcagctgctgtgtttgtgggtggtccaaactaatgagtgacccagccaaacttgtaaatcacagggctgcagtccttctgagtgggggtgggacatcctgggacagaaattcactgcctgggagatcatcaggctgtgggggaaagccctggcctttgggggccctggttcaaagccaggaactgggaggtggaacagctgccactggtcagcaagtgtgcttgtgacagctgtcagaagcctcgccctcactctgtggcccccacctaaggctgtgctgccgctggtcagaagccttgtcctgactccatggcccctaccctaggctgtgctgagagcggaggggtccgtcagagtccccagaagcccatccccctgctgctttcccccacgcagagcccagtcccttctactctgagttctaatctgatttcagaaaagcactgggctcagacacagagtgtgagactgagcctttcttatgggagttgtgtgcctggtggtcctagagacaccctcgagcttctctgagtgatgctttgggacggtgggtagggaagcaccctgcgtggtgtgtggtgcccgtggacatcctgtgagtgactgctgttcatgtgggtgatgtggtcacacttgctcagggtctgttctgcagcccaggttgacacctgttactccagcctggttatgagcattgttctgatctgttctcaacattgtccaggtttgggagataaatgccagaggagagtctggttgggggctcccagagctcacatctggggtgtgttggtctgcagcctggtagtggtaatggctccctcggtagctggttgtgtgtgcaggtgtccgcggcagctgtacgtgcaggtgggtggactgagctgagtgtgaggatggtgggagaaggccttggtgacggtggcagtgctgccacctactgagcacctgctgtgtggtatgcgggctgatgtcggcttgcaccgcagtcccaggactgggccttgtaatcccattttaggaagaggagcccgaggctcagggtgctggtgcagagccccctggctagtgagcatcagggctgcggtatgtacttgagtatggttctagcaccttcccccggagcgtgagtgcgtggcaggtgctgtcactgtggctgagttagctgctccgcttccccaacaggctggcggagctcgtgcccttcgtcaccgtgaacgcccagtggggcatcctcagcaagtgcctgacctacagcaaggcggtggccgacccgttcacgtactctctgctccgccggccgttccgccaagtcctggccggcatggtgcaccggctgctgaagagaaccccgcgcccagcatccacccatgacagctctctggatgtggccggcatggtgcaccagctgctgaagagaaccccgcgcccagcgtccacccacaacggctctgtggacacagagaatgattcctgcctgcagcagacacactgagggcctggcagggctcatcgcccccaccttctaagaagccctgtggaaagggcactggccctgccacagagatgccactggggacccccagacaccagtggcttgactttgagctaaggctgaagtacaggaggaggaggaggagagggccggatgtgggtgtggacagcagtagtggcggaggagagctcggggctgggctgcctggctgctgggtggccccgggacagtggcttttcctctctgaaccttagcttcctcacccttgttctggggtcatggcgatgcttcgagacagtgggtagggaagtgccctgtgtggcatatggtactcgtgggcgtgctataagtgactgctgttcatgtgggtgaggtggtcactcttgctcagggtctgttgtgcagcccagatggacacctgtttctccaacctggttattagcattgttccgatttgttctcggcattgcccaggtttgggagataaatgccggggcggagtctggttgggggctcccagagttcacatctgatagtctgtggtcaggacctggcaggcacgggcagtccctgggacatgcccatctctggaagcctaggggtccccagctccaggcctgtccgctgtgactgcctgtgtgggcacgcagatggagcctgtctcctgccttcctttccatggtttgccaggggtttggcatcttgactgcggaagctgtggagtctgtgtgctcagagccttttctggtgaagatatcatcagagcatgtgacctctgtttcctccccctgaaggccaccgctgggcctctggatcttagacatgagacggtcaagagattgaagtagtagccagggcccaggtgtccagagagggtggcctgggatggggagggcccttgctccccaacagcagtgctgggggagccaagagaaggtggagcatccctgagtagtggtgtgcatcacccccagtttagtaatcacggggtgccattccccggtgggagcacccaccatcaatgtcattgaatgtccccatgggacagtgttgaggacttttgtgacatctgtcctatttcacagctcagggaaaggtgcacagtgcacacgggcacccggtggagaggtgtgtgtgtgaatgagtgagcgagtgaatgaatggacacgattctctcttcagcctctgtcattgctgttttcttcaaggcccagggccatcccctgcagaggtagggtgggctgcaagacctcaggcccctgcctcatgggactctctgatgggcttcaaccgtgggctcttgcaggcatggagcctgtatcatgacaccttacacccaaggccagcaatgcaaggagagtatggacatcaaattctttccttccagaggctgaattcttcaaagacacacgcggtcgtcccttgctcttggcattaacggtggagaacccagctgaggtggcttcacagattcttccccaaaaacacaggtgttattattatacttttaaaaaactttttgagacagggtctgactctgttgcctaggctagagtgcagtggtgcaatctcagctcactgcagcctccacctcccatgctcaagccatcctcccacctcagcctcctgagtagttgaggacacaggcacgggacaccatgcctggctaatttttgtatttttttttgtagagatggcggtctcactttgttgcccaggctggtcttgaattcctgagcttaagtgatcctcccacctcagcctcccaaagtgctgggattacaggtgtgagccaccacacccagccaaaaccaggtgttatttgctgactcaccaatgcctcccccaaaaggataaatttaaaggtgtgtataactcatgaagtgtaattcaaataaaacaaattatcgtttggaataataacaactataggtatgtggtcaggaagcagttaaaaacattaaaatacagacctggccagttcaatccaacccaggggttccaagcaggaggtggggcaggtgggcgtcatgccctgattcacagagggcacaggtgggtgtcatgccctggttcacagagggcacgtgaactcgagaccgtgctgcaccccggtgcccctgtgcttataagggagggcacgtgcacagcagaagcaggttgttccccatttaaagttctggagcccaggctgtgagctccttggctgagccctctcctgtccctgggagctccccaggtgcgaggagcctgccagccagtggggcctacactctgtgttattgcatctccgccaggctaaaagccttggtcactactttagagccactcaaggaaacgcgtgcaccctgccctgctggaaggcaccatggttagagggaggcacactgtttcttagagacggggactgcttgctgtcatgtttcgccttcctcggaagctccatggaatgttctggagcaggcatcttagggcattccctccgcacttctctgccagcccatgtggctcccacactgggctatcccttgccttaggcttgtggcctttttttttttttttttttaatttgaaaaatatttttcatgtgcacttaaacgtgttgtggaatgatgctgggtctcaagaatgctgtgaatcaataaacattttattcagagggtgtctcatttccatggaaaggggggaattctgccgtgcattagccacagtccaattaccaggatagcagagctttctagatgcctgggcttgcaggcaatcccctgctgggcttccaagacctccaggctaggccctgcactgtgtgcagcgagcgagggagccttctcctgttgttttgacggtggagctgcaagcttgggaggctttcccgtgggtgggcgggaagcagagagccgtgggggctgtgaggctggcagggagcttgcaggacataactgctcatttattttatttgggggaccggagtttttttattgattccatctgtcagagggctgcagcattccatgacgggtaaggcagagggaggccctgtgggagcccagagtgacagtcctctctctcagcttgccttcttgtccttttgagaccatgatctgcttctcctgagcctccctcctgttcacatgggatacaaaactcagccagaaggggttctgagccaggagccctctctccatcccctctgtgcttgctgaccctcgccctcctgcctcacctgggcacccatccgtagccaagcctccagaacccttcctggtccctgtcgaagcagtccttcctctcacccaccctccaccaagcagagccagctcctagaaatgcagctgagagcaggttgtgttcctgccagaagccctgagaagtccccaggctatggtctctgctcctgagcctggcattgctggccctttctgttctctgtcctcactgctccccactcctatctacgtggcctcgggctaaagtactgggtgccctagtctactggcctctccccagggggcctggatcctttctgggccttctcttggtacttcctcctcctctgcttagccaagtccttccacctgactgaggcagacacaaccctccctcctcaggtgctcacagccccagcacccttctctcaagggtccagccatagggtgggtaggaacagggtcagacccacttttatccccagcacccgctctgcagcaagtgctggggatgccggcatgcacacaccatgtccagctggggagcaggcttccaacgactgacgggctcctccggctgccatgctgaccctaccccaacctcatgagccccaaggtgggccgctcttttgagcggtgagtctgtgctcttttgatgacacaaagtggcttctctcctagaaacttgtcctgcagggcaccccccatgggaccacacacccggccccaggggctgtgctcttgcagacttggctctggccacacctgctggttcactggccccaccttgactgcacaggtgtggctgggtcctctccccgtggcctattgcccgctgggctggctggacagtgtctggggatttggtgtgatggggacgacttggatgcctcttttacccctgggaacgattaaccctgtcttgtgatttgcccctgatgaaccatttggtgggttgggccctcgtgggtgagatgctgtggatttgaggctgtcccttccaacttaactgtggaagagagaagctggttccaggaaagatgaccaaattcttctccccgagtcttaaaaagtgacacaagctagcactcaatttcctcaaatctaaaatgccgttgattttaaactgtctgttttaggtaccactgggaaggaaaagcccagaatgagggctgtgctaggagacaccctcaacaggtgggccactcacggctgaacgctcagcgtccacgagaactaagcctgccacagcaggggacaacaaggaggcacggtgtccccaccccagctctcagggaggaggaggaaaaccctccctgggactgtgcactgccagctggggctcgggaaagcatggagtctgaattcgccctcagacctgggctggaaagctcagacagggaagtcaaagactgtggccccggaggctggccggggcagtcagaggtgcttctggaaggacccagctgagtccaggcagagagagggcaaggttgagcaccaggcgccccagatcccggggggtattgaaatgggcatctttgagcagatgacctgcaggaagcagcacctgctgggtgccagcaatgagttggggccacagtgagtactgtctccattaccccagcctctgggcgaaggggcttgtcctaagtctcagggctggctgagtggcagctggctctggggactgctactggtgcactggtatagctggcaaaggaacccatgaggcacatggctcctaaaggtccagccacccagcaaagccccctccccgcatccacacaggggacaagggtcaaaaggtggggacatgccttcactttcctcacctgacaggccctggtctgctggggtcagcgctgcagccagaacccgcattcacccgcgacgcagcctgtgcaggggaccaggggttttaggcaggatcagcagggaatctgcataccagctccacagtgcactcaggtggcagatggggaaactgaggcccaaggaggggcggggagccccttggacggggcggggcactccgcccggggcaggcaggggcctttatctgctgtcctgcccctcctcctcccccagcggcatcctctctctagcttctggcgctgcccactgtaacccgactccggcatttgcgtttggggcgccctccctgcgccgggggcgggagcccagcgagcgcagagccccggccccgcgcggcccgagtgccacatcactgcgctggccgtccaaggtccgccgccccaccatgccgcccccgccgccgctgctgctccttacagtcctggtcgtcgccgctgcccggccggggtgcgagtttgagcggaaccccgccggtaaggccgtcccctgcccccaccctccaccctctaccctgcaacctctggggagtgtgatccgtcgcttcccaggggcccgggagctttcttccagtaggtcacgcgcctatggtcctggcgagaacgtctccaaagtgggcagcatgtggcctgggactggtagggtgacctctcccctggacggggacaccagggaggctctctacggcatccaggccgggaccccaggcagcagagggtcaccacgcctgggccggggggtgagatctttttcctgtcggacacagcagcaggggcgccctgcaggtgggttcgagggagcaggtgccaggactttgcagggtgagaggccatctaaggtccccgggtctttccaggaacgggacagtctctctgggctgtggcaaactcttgacccctccctcccagctggggctgtgatgtggacagcaagtctccggaagtggccctaagggctgggagggggcgtgggccccctggagggatctggggcccggagagggaatggacgcagggatcctctgggaggtctgcccgcacgacctcacccaaggggtgtgcagcgggggtggggagcaggaaggtagaggctgggggctggatcctgggctcccgtctggcctcacggcctctccaggtgtgggggtgcatctagccaaccgtgctcaggactctcatcagaagcagacatctgggctcctgcgggagtggggccgtgtgtgggttcaagggtgcagggtgtcaggttctgaatcgcttgttctctggggtttgtggatacctgagaactgcctgagccctgcgagtggatgtgccaggaccacaataccccaccccagtgcgctgtgtcgacacttttcctttccgtcctgttgatgcctggattttcctcccacctctgctcctcgccttacctctgggctcttttctacccgctgcgtgtgtgctcatgcaggtgtgcaggtggggtgctgctggagcttgtgccgtgttgtgggtgggcctccccttggcccctgagagcccagacagtatctagaatcataggcttgttggagaccacagccccctcctccaggaagctctcctgacctgccctttcccaggaagaaatgcagcctcctcctctgcacgcaggcagcacagaccaccctgtcctcacccagcaggatgcatgggctggtctctgtccctggcaggtgctgagcacagccgtggcatcggcaagtgtttctgggagaaatcactcatcccccagtccagtctcccctcttatggacgagtgtggaagtcaaggacgtttccagcccacaggcagaagtgggcagagccggtcacctgcagtgcaggtcccccaccccgggctgccatgtccctgtctcgccatctgggtcttgctgagagcaagcctggtgctctctcctctctgcctcacccttccctggtggaagattccctgtcctcactggagcctggggacggaggtaattttcatgtcctagggtctgggattcagattctgactcctcaactctgctgtgtgacctgggcagatggcctgacctctctgacctcattcaggtctcatccctgacccaggcacagccactggtcagtttaggagggggaggactgacgggctgtcactcccctgttgaagaaatgctgccacctcgtggttaagaggcttagaactatttcaaaagccgctttcagaccagtgctgtccaacagaaacacaaagccatccacgcacagaattttatattttctagtagccacactaatgaggtaacagaaacaggcgaaactgattttaataacaaattcttttttttttttttgagatggagtctcactctgtcgcccaggctggagtgcagttgcgcaatctcagctcactgcgagctccacctcccgggttcacgccattctcctggctcagcctcccgagtagctgggactacaggcatctgccaccgcgcccaactaattttttgtagttttagtagagacagggtttcaccgtgttggccaggatggtctcgatctcctgacctcgtgatccacccgccttggcctcccaaagtgctgggattacaggcgtgagccaccacgcctggcctgtaacagattctatttaacccaatatgtccaaaatattccagcattttggcattcatataaaaattattaatgaaacacactgtttttagtactaagtctgaaatttggtatgcatttcacacatcgcacacattggtttgatcttgccacatgtgaagggctcagtggccacgtgtggctcgtggctgctgtactggagaccatggcttgagagccttctaggggcataggctttcaccccactgtgcgtttgcaagtctgcagggggatccggggatgtctgtgtcccacggtggttggggtggggaagcaatggtgaatagcctaaccagccacagttgtcctggctttgctctgtggctgccagggcaggtgcggcctgggagaggcagagggtgggctttgggtcagcaggcccacccccgtgtggtaaggggcaaaaccagaggcctacggaggccaaggcagctggcaaaggcccagccacatcatgggactagctgtgtggtcttggggcagttgcagcccccgagggctcctcccagaccatcctgactctcttgtggccacagcagcacccactggggcttgctgattgttgggggaagaattgtgttttccagaaaagacatactgatgatgtaatccctggcacctgtgaatgggagcgtacttggaaacaaggtctttgtggatgaaatctgattcagatgaggcctcacgggattagggcaggccctaagccaataactggtatccctagaagaagagggagatttgggcagagacagacacagggacaaagttcacacatgacaacagaggcagggactggagtgctctggccacgagccaaggagcacctggggccaccaggagccgggaggggcgggagagttcttcccttagagccttgacagggagtgcggccctgcagacctgcagactttccaagcctccagggctctgaactgttgttgcttttttttttcaagacagagtctctctctgtcacccaggctggagtgcagtggtgtgatcttggctcactgcaacctccacctcccaggttcaggtgattctcccacctcagcctcccgagtagctgggactacaggtgtgtgccaccatgcccggctaatttttgtatttttagtagagacggggtttcaccatgttggccaggctggtcttgaactcctgacctcgtgatccacccgcctaggcctcccaaagtgctgggattacaggtgtgggccactgcacccggctgttgttgatttaacctaagcatggtgggcgctttgctagggcagccctggcaaacaaacccacccctctctgtgccttgcacatggcggccagtggaagtgggggtgacccagccagcagacactcttcctgccccttgggaaaccccgatggggctgcatggcttattgtggggtcacaggggatagtcctgctcctgcccacgatatgccccaagactctgtgtgttgagcattcactgggcacctcaccctcctgttgtattatcttgatggatcctccaacagccctatgaggtagacgtgatccttatcccaatctacacatgaggaaactgaggcacgggcagtggttcatcctggagtcttagtgccctcatctgtgaacaagggagactggaagccacaggaagccaggaaggatcgcctgtccagtccctgtgtgatggtccagtcacttgtgtgggcgcttggtggctttggaggagcaggtgcagggatggacacctcaccttgtagctccctgaggccagcagagttcccagggtcaagtcaaagttagttcttccagtcgctcatgtctgctgagtgaataaacaaagttccaggttcacccaagcttgccagctcagggccaggccacgctcagtgccagccgggcaccgtcagagccttgtgatgggtacccagggagtggagcaggggtgctgggctgagatcaccttgacccttgagctgactgtgctgtagcatctgcctcggtccaagctcagtgcaggatgagaccacgggtcagctgagtgcaaaccctgctgccagagtggccccactggtggccagctttgcacaccggtgctcgctcagggccctgcacaggatgggtgctcacacagggccctgtgtggatgccagccttttatctgctcttcccaacatcacccagttgtctttagccaca

SEQ ID NO: 4 - protein, 5 aa, homo sapien
MGPGE

SEQ ID NO: 5 - protein, 12 aa, homo sapien
RGRTPSAPGACQ

SEQ ID NO: 6 - protein, 24 aa, homo sapien
SSAFASCSLRLPPEPERPRFAAFT

SEQ ID NO: 7 - protein, 14 aa, homo sapiens
RLAELVPFVTVNAQ